SEQUENCE LISTING (9 sequences)

SEQ ID NO: 1 - protein, 185 aa, Homo sapiens
MKLVSVALMYLGSLAFLGADTARLDVASEFRKKWNKWALSRGKRELRMSSSYPTGLADVKAGPAQTLIRPQDMKGASRSPEDSSPDAARIRVKRYRQSMNNFQGLRSFGCRFGTCTVQKLAHQIYQFTDKDKDNVAPRSKISPQGYGRRRRRSLPEAGPGRTLVSSKPQAHGAPAPPSGSAPHFL

SEQ ID NO: 2 - protein, 48 aa, Homo sapiens
ELRMSSSYPTGLADVKAGPAQTLIRPQDMKGASRSPEDSSPDAARIRV

SEQ ID NO: 3 - protein, 212 aa, Homo sapiens
MDYLLMIFSLLFVACQGAPETAVLGAELSAVGENGGEKPTPSPPWRLRRSKRCSCSSLMDKECVYFCHLDIIWVNTPEHVVPYGLGSPRSKRALENLLPTKATDRENRCQCASQKDKKCWNFCQAGKELRAEDIMEKDWNNHKKGKDCSKLGKKCIYQQLVRGRKIRRSSEEHLRQTRSETMRNSVKSSFHDPKLKGKPSRERYVTHNRAHW

SEQ ID NO: 4 - protein, 195 aa, Homo sapiens
APETAVLGAELSAVGENGGEKPTPSPPWRLRRSKRCSCSSLMDKECVYFCHLDIIWVNTPEHVVPYGLGSPRSKRALENLLPTKATDRENRCQCASQKDKKCWNFCQAGKELRAEDIMEKDWNNHKKGKDCSKLGKKCIYQQLVRGRKIRRSSEEHLRQTRSETMRNSVKSSFHDPKLKGKPSRERYVTHNRAHW

SEQ ID NO: 5 - protein, 45 aa, Homo sapiens
RSSEEHLRQTRSETMRNSVKSSFHDPKLKGKPSRERYVTHNRAHW

SEQ ID NO: 6 - protein, 116 aa, Homo sapiens
APFRSALESSPADPATLSEDEARLLLAALVQDYVQMKASELEQEQEREGSSLDSPRSKRCGNLSTCMLGTYTQDFNKFHTFPQTAIGVGAPGKKRDMSSDLERDHRPHVSMPQNAN

SEQ ID NO: 7 - protein, 57 aa, Homo sapiens
APFRSALESSPADPATLSEDEARLLLAALVQDYVQMKASELEQEQEREGSSLDSPRS

SEQ ID NO: 8 - protein, 32 aa, Homo sapiens
CGNLSTCMLGTYTQDFNKFHTFPQTAIGVGAP

SEQ ID NO: 9 - protein, 21 aa, Homo sapiens
DMSSDLERDHRPHVSMPQNAN

(19) United States
(12) Patent Application Publication
(10) Pub. No.: US 2022/0187313 A1
(43) Pub. Date: Jun. 16, 2022

(54) Diagnosis or Prognosis of Postsurgical Adverse Events

(71) Applicants: B.R.A.H.M.S GMBH, Hennigsdorf (DE); LINKÖPING UNIVERSITY HOSPITAL, Linköping (SE)

(72) Inventors: Michelle Chew, Linköping (SE); Henrik Andersson, Linköping (SE); Darius Wilson, Bearsden (GB)

(21) Appl. No.: 17/432,049

(22) PCT Filed: Feb. 21, 2020
(86) PCT No.: PCT/EP2020/054652

§ 371 Date: Aug. 18, 2021

(30) Foreign Application Priority Data
Feb. 21, 2019 (EP) 19020082.4

(51) Int. Cl. G01N 33/68

(52) U.S. Cl. CPC G01N 33/6893; G01N 2800/52; G01N 2800/26

(57) ABSTRACT

The invention relates to a method for the diagnosis, prognosis, risk assessment and/or risk stratification of an adverse event in the health of a postsurgical patient, comprising providing at least one sample of a patient, who has undergone, is undergoing or will undergo surgery, wherein the at least one sample has been isolated from the patient presurgically, perioperative or postsurgically, determining a level of at least one biomarker selected from the group consisting of proADM, PCT and pro ET-1 or fragment(s) thereof in said at least one sample, wherein said level of the at least one biomarker or fragment(s) thereof correlates with the likelihood of said adverse event. In some embodiments the method is used for predicting or diagnosing an elevated or decreased likelihood of a postsurgical infection or blood infection. In some embodiments the method is used for predicting or diagnosing an elevated or decreased likelihood of a postsurgical adverse cardiovascular or cerebrovascular event, preferably a major adverse cardiovascular or cerebrovascular event (MACCE), major myocardial injury after non-cardiac surgery (MINS) or perioperative myocardial injury (PMI). In some embodiments the method additionally comprises therapy guidance, stratification and/or control in relation to said postsurgical adverse events, in particular an infection or an adverse cardiovascular or cerebrovascular event.

The invention relates to a method for the diagnosis, prognosis, risk assessment and/or risk stratification of an adverse event in the health of a postsurgical patient, comprising providing at least one sample of a patient, who has undergone, is undergoing or will undergo surgery, wherein the at least one sample has been isolated from the patient presurgically, perioperative or postsurgically, determining a level of at least one biomarker selected from the group consisting of proADM, PCT and proET-1 or fragment(s) thereof in said at least one sample, wherein said level of the at least one biomarker or fragment(s) thereof correlates with the likelihood of said adverse event. In some embodiments the method is used for predicting or diagnosing an elevated or decreased likelihood of a postsurgical infection or blood infection. In some embodiments the method is used for predicting or diagnosing an elevated or decreased likelihood of a postsurgical adverse cardiovascular or cerebrovascular event, preferably a major adverse cardiovascular or cerebrovascular event (MACCE), major myocardial injury after non-cardiac surgery (MINS) or perioperative myocardial injury (PMI). In some embodiments the method additionally comprises therapy guidance, stratification and/or control in relation to said postsurgical adverse events, in particular an infection or an adverse cardiovascular or cerebrovascular event.

### BACKGROUND OF THE INVENTION

Worldwide, more than 300 million adults aged 45 or older undergo major noncardiac surgery each year (Weiser et al. 2015). Out of these, millions die from complications within 30 days (Botto et al. 2014) and a significant proportion of patients suffer from major cardiac complications during the first year after surgery (Ekelof et al. 2016). The prime cause of postoperative mortality is cardiac complications due to ischemic injury of the myocardium, causing cell damage and uncontrolled cell death (Botto et al. 2014, Sessler et al. 2018, Landesberg et al. 2009). In addition to the number of deaths, postoperative cardiac injuries result in a substantial number of postoperative complications prolonging recovery and hospitalisation, which results in increased patient suffering and medical costs (Devereaux et al. 2015).

### Postoperative Major Cardiovascular and Cerebrovascular Events (MACCE)

Major Cardiovascular and Cerebrovascular Events, MACCE, comprise the most common causes of serious perioperative morbidity and mortality and depending on the population studied the incidence ranges between 1-7% (Sabaté et al. 2011).

10% of patients who develop MACCE die during their hospital stay. Several independent preoperative and intraoperative risk factors for MACCE have been suggested, e.g. coronary artery disease, chronic congestive heart failure, intraoperative hypotension, and blood transfusion. Risk stratification systems with a high sensitivity and specificity are however missing.

Myocardial Infarction (MI) after Surgery

Myocardial infarction may be defined by a necrosis of the myocardium caused by prolonged ischemia and is a common cause of death and disability (Thygesen et al. 2012). Two distinct mechanisms may cause myocardial infarction, both of which are seen after noncardiac surgery. A type I MI is caused by an acute coronary syndrome; a rupture, ulceration, erosion or dissection of an unstable atherosclerotic plaque in one or multiple coronary vessels with subsequent intraluminal thrombosis leading to reduced myocardial blood flow (Landesberg et al. 2009, Devereaux et al. 2009). A type II MI is caused by a prolonged imbalance between myocardial oxygen supply and demand and may arise as a consequence of conditions such as cardiac arrhythmia, anaemia, respiratory failure, hypotension, hypertension or by direct toxic effects by high levels of endogenous or exogenous circulating cortisol and catecholamines (Ekelof et al. 2016, Landesberg et al. (2009), Thygesen et al. 2012).

The diagnostic criteria of MI consist of an elevation of the cardiac biomarker troponin, greater than 3 times the upper level of the reference range in the setting of suspected myocardial ischemia with either an ischemic symptom or an ischemic electrocardiographic (ECG) finding (Thygesen et al. 2012).

The ischemic symptoms of MI include various combinations of nonspecific symptoms such as chest-, upper extremity-, mandibular pain, and dyspnoea. Symptoms often last >20 minutes, are diffuse, not localized, positional or affected by movement.

However, MI-patients are often asymptomatic. This is particularly the case for post-operative MI, wherein myocardial injuries often occur within 48 hours after noncardiac surgery (Thygesen et al. 2012). A possible explanation for the lack of symptoms during this period is that the patients usually receive analgesic medications that mask the symptoms. This may partly explain why 65% of the perioperative myocardial infarctions (PMI) are asymptomatic and associated with poor outcome (Devereaux et al. 2015, Devereaux et al. 2017). 12% of patients with PMI die within 30 days and most deaths occur within 48 hours (Hanson et al. 2013).

Myocardial Injury after Noncardiac Surgery (MINS)

MINS is a prognostically relevant myocardial injury due to ischemia occurring in 8% of surgical patients (Botto et al. 2014). 10% of patients suffering MINS die within 30 days, MINS-patients also have a greater 1-year mortality as well as beyond 1 year and a greater incidence of nonlethal cardiac arrest, congestive heart failure, and stroke (Botto et al. 2014, Mauermann et al. 2016). Furthermore, the regional myocardial injury can stun the myocardium, resulting in diastolic dysfunction and reduced cardiac output resulting in reduced peripheral blood supply, impaired healing of tissue and increased risk of wound infection (Noordzij et al. 2015).

A current diagnostic criterion for MINS is a postoperative troponin elevation judged to be due to myocardial ischemia (no evidence of nonischemic etiology) with or without ischemic symptoms. MINS preferably does not include perioperative myocardial damage originating from another etiology than ischemia, e.g. sepsis or pulmonary embolism. The criteria for MINS covers a wider spectrum of myocardial injury than MI, from reversible myocardial injury to necrosis. This broader concept comprises several manifestations of myocardial injury and makes MINS a more applicable term for surgical patients and lowers the risk of underestimating the extent of myocardial damage seen after noncardiac surgery (Mauermann et al. 2016).

93.6% of MINS occurs within 3 days of surgery. MINS patients will have mild troponin elevation and often lack ischemic features (Noordzij et al. 2015). 84% of patients suffering from MINS do not experience ischemic symptoms and only 34.9% will show signs of ischemia on an ECG (Botto et al. 2014).

These findings indicate that a reliable diagnosis of perioperative myocardial injury, in most cases, is only possible by continuous postoperative troponin measurements. Otherwise, it is reported that 93.1% of MINS and 68% of PMI may go undetected (Devereaux et al. 2017).

Moreover, due to a delay in the onset of troponin in case of cardiac complication, a troponin free window exists, which impedes a prompt initiation of therapy and raises the likely of severe complication and mortality.

The mechanism of MINS remains unclear but is suggested to be similar to the mechanisms behind PMI (Mauermann et al. 2016). Several trials have been conducted trying to counteract MINS but there are still no known methods for safely and efficiently preventing the myocardial injuries (Sessler et al. 2018). One example, the POISE-study, a large randomised trial administering beta blockers perioperatively and postoperatively, managed to reduce the occurrence of major cardiovascular events by 30%, but at the same time increased the incidence of serious hypotension, stroke, and mortality (Devereaux et al. 2008). Due to the difficulty of detecting or predicting MINS based on clinical symptoms, clear diagnostic and prognostic means based on molecular approaches are needed.

### Perioperative Myocardial Injury (PMI)

Perioperative myocardial injury (PMI) is a serious contributor to mortality after noncardiac surgery. Because the vast majority of PMIs are asymptomatic, PMI usually is missed in the absence of systematic screening. In particular, PMI is a common complication after noncardiac surgery and despite early detection during routine clinical screening, is associated with substantial short- and long-term mortality. Mortality seems comparable in patients with PMI not fulfilling any other of the additional criteria required for spontaneous acute myocardial infarction versus those patients who do (Puelacher et al 2015). In light of the difficulties in detecting PMI, and the difficulties in predicting whether a patient is at risk of PMI, additional means are required to address these difficulties and enable suitable, preferably early or preemptive, therapeutic or prophylactic measures.

### Postoperative Infections

The development of an infection after surgery constitutes one of the major postoperative complications and causes pain, poor wound healing, a need for further treatment including antibiotics, longer hospital stays and increased health care costs. Postoperative infections may cause severe problems including failure of the surgical procedure, sepsis, organ failure and in some cases ultimately death.

A postoperative infection may be defined as any infection that occurs within 30 days of operation and may be related to the operation itself or the postoperative course. The incidence of postoperative infections can vary widely and depends on the type of procedure performed and the patient's inherent risk factors for the development of infection.

Postoperative infections can for example involve the wound itself or deeper infections within body cavities or systemic infections, but may also include more distant infections such as pulmonary infection (e.g. pneumonia) or catheter associated urinary infections.

Postoperative wound infections, also known as surgical site infections (SSIs), complicate the recovery course of many patients. As defined by the Centers for Disease Control and Prevention (CDC), these infections typically occur within 30 days of an operation at the site or part of the body where the surgery took place.

SS's may be classified as superficial/incisional if limited to the skin and subcutaneous tissue, as deep incisional when involving the fascia and muscle, or as organ space when involving a body cavity (e.g. in the abdominal cavity following gastrointestinal surgery) (Horan et al. 2008.)

Deep tissue and organ space SS's are less frequently encountered than superficial SSIs, but are associated with greater morbidity/mortality, readmission rates, longer hospital stay and increased overall hospital-associated costs when compared with superficial SSIs. Although the majority of SS's are uncomplicated, others may be severe and more challenging, often requiring extensive surgical debridement, multiple reoperations and may be life-threatening.

Postoperative infections may arise to a number of factors in addition to an infection at the surgical site. Post-operative pneumonia, and respiratory tract infection are for instance generally regarded as a manifestation of physiological and immune compromise, which occurs after major surgery. Postoperative pulmonary infections are associated with cough, phlegm, shortness of breath, chest pain, temperature above 38 degrees C., and pulse rate above 100 a minute. Up to half of people may have asymptomatic chest signs after surgery, and up to a quarter develop symptomatic disease.

The incidence of postoperative pulmonary complications in major surgery ranges from <1 to 23%. Several studies have shown pulmonary complications to be more common than cardiac complications, and postoperative respiratory failure is a common complication postoperatively (Miskovic at al. 2017).

Diagnosing a surgical site infection can be challenging, as the signs and symptoms are frequently nonspecific and may lack sensitivity depending on the clinical scenario.

However, an early diagnosis of postsurgical infection is of critical importance in order to initiate appropriate therapy such as antibiotic treatment.

A particularly severe postsurgical infection relates to a blood infection or sepsis. Despite significant improvements in diagnostic and preventative measures, the incidence of sepsis has continued to escalate rapidly in hospitalized patients (Martin et al. 2003), with mortality rates ranging between 10% and 54%, depending on the level of disease severity, definition of organ dysfunction used, and country specific incidence (Kaukonen et al. 2014, Vincent et al. 2006).

For any postoperative disease, but in particular for sepsis, an early and accurate assessment of both the infectious load and disease severity, in terms of the overall pathophysiological host response, is of crucial importance in order to make prompt and reliable decisions concerning diagnostic testing and treatment strategies.

In light of the prior art, a need exists for the provision of diagnostic and prognostic methods that may allow for an early diagnosis or risk assessment a postsurgical adverse event such as a postsurgical cardiovascular or cerebrovascular event or a postsurgical infection.

### SUMMARY OF THE INVENTION

In light of the difficulties in the prior art, the technical problem underlying the present invention is the provision of improved or alternative means for the diagnosis, prognosis, risk assessment and/or risk stratification of an adverse event in the health of a postsurgical patient, in particular within the first days after surgery.

The present invention therefore seeks to provide a method, kit and further means for the diagnosis, prognosis, risk assessment and/or risk stratification of adverse events, including in particular postsurgical infections or cardiovascular or cerebrovascular events.

One object of the invention is the use of a biomarker or combination of biomarkers to distinguish preoperatively, perioperatively or postoperatively whether or not a patient has suffered or is at risk of suffering an adverse event after surgery.

The solution to the technical problem of the invention is provided in the independent claims. Preferred embodiments of the invention are provided in the dependent claims

The invention relates to a method for the diagnosis, prognosis, risk assessment and/or risk stratification of an adverse event in the health of a postsurgical patient, comprising
providing at least one sample of a patient, who has undergone or will undergo surgery, wherein the at least one sample has been isolated from the patient presurgically, perioperative or postsurgically,
determining a level of at least one biomarker selected from the group consisting of proADM, PCT and proET-1 or fragment(s) thereof in said at least one sample,
wherein said level of the at least one biomarker or fragment(s) thereof correlates with the likelihood of an adverse event.

The invention relates in particular to a method for the diagnosis, prognosis, risk assessment and/or risk stratification of an adverse event in the health of a postsurgical patient, comprising
providing at least one sample of a patient, who has undergone, is undergoing or will undergo surgery, wherein the at least one sample has been isolated from the patient presurgically, perioperative or postsurgically,
determining a level of at least one biomarker in said at least one sample,
wherein said level of the at least one biomarker or fragment(s) thereof correlates with the likelihood of said adverse event, wherein the at least one biomarker is proADM or fragment(s) thereof and the adverse event is an infection.

The invention relates in particular to a method for the diagnosis, prognosis, risk assessment and/or risk stratification of an adverse event in the health of a postsurgical patient, comprising
providing at least one sample of a patient, who has undergone, is undergoing or will undergo surgery, wherein the at least one sample has been isolated from the patient presurgically, perioperative or postsurgically,
determining a level of at least one biomarker in said at least one sample,
wherein said level of the at least one biomarker or fragment(s) thereof correlates with the likelihood of said adverse event, wherein the at least one biomarker is PCT or fragment(s) thereof and the adverse event is a cardiovascular or cerebrovascular event.

The invention relates in particular to a method for the diagnosis, prognosis, risk assessment and/or risk stratification of an adverse event in the health of a postsurgical patient, comprising
providing at least one sample of a patient, who has undergone, is undergoing or will undergo surgery, wherein the at least one sample has been isolated from the patient presurgically, perioperative or postsurgically,
determining a level of at least one biomarker in said at least one sample,
wherein said level of the at least one biomarker or fragment(s) thereof correlates with the likelihood of said adverse event, wherein the at least one biomarker is proET-1 or fragment(s) thereof and wherein the adverse event is preferably an infection and/or a cardiovascular or cerebrovascular event.

The patients of the method of the present invention have undergone, are undergoing or will undergo surgery at the time of taking the sample. The likelihood of the occurrence of an adverse event in the health of the patient after surgery can be assessed upon the level of the biomarkers proADM, PCT and/or proET-1 prior to surgery, during surgery or after surgery.

The method can be very useful for prompt therapy guidance of postsurgical patients. By determining the level of the biomarkers proADM, PCT and/or proET-1 the likelihood for an adverse event such as an infection or a cardiovascular or cerebrovascular event can be determined independent of the manifestation of clear clinical symptoms.

The likelihood of the occurrence of an adverse event after a surgery can be assessed on the comparison of the level of proADM, proET-1 and/or PCT or fragments thereof in the sample in comparison to a reference level (such as a threshold or a cut-off value and/or a population average), wherein the reference level may correspond to the level of proADM, proET-1 and/or

PCT or fragments thereof in patients that have not suffered a subsequent adverse event after surgery and patients that have suffered a subsequent adverse after surgery. Optionally the reference level may correspond to the level of proADM, proET-1 and/or PCT or fragments thereof in healthy subjects.

Accordingly, the method of the present invention can help to diagnose or predict the likelihood of an adverse event in the health of a postsurgical patient. This means, that the method of the invention can discriminate high risk patients, who are more likely to suffer from complications, or whose state will become more critical in the future, from low risk patients, whose health state after surgery is stable or improving, so that it is not expected that they have suffered or will suffer from a postsurgical adverse event, such as an infection or a cardiovascular or cerebrovascular event, which might require certain therapeutic measures.

For postsurgical patients the circumstances of the surgery often mask early clinical symptoms of an adverse event. Typically, during the first days after surgery, but possibly also for an extended period of time, patients routinely receive a number of medications, including analgesic medicaments and pain relievers such as nonsteroidal anti-inflammatory drugs (NSAIDs), which attenuate the perception of the patient or a clinical personal in regards to possible early clinical symptoms of an adverse event.

For instance, chest pain, shortness of breath, nausea, or a feeling of faintness constitute clinical symptoms of a myocardial infarction. A stroke is typically accompanied by residual motor sensory, or cognitive dysfunction. However, under the influence of the residual anesthesia or additional pain reliever medication such symptoms may be either not be perceived at all or misjudged. Similarly, early signs of infection such as headaches, local pain at the site of infection, weakness or general non-wellbeing may be mask by the medication or misinterpreted as routine consequence from the surgery.

As the data below show in more detail, determining the level of the biomarkers proADM, PCT and/or proET-1 allows for a prognosis or diagnosis of adverse events independent of clinical symptoms and may thus constitute a valuable guidance for therapy and monitoring.

For instance, the early diagnosis of an infection using the method described herein allows for a prompt initiation of appropriate antibiotic treatment. Likewise, diagnosis of a cardiovascular event may allow for a prompt initiation of coronary artery bypass grafting, administration of appropriate medication or coronary angioplasty.

It was a surprising finding that the biomarkers proADM, PCT and proET-1 show a similar potential as markers for predicting or diagnosing adverse events in the health of postsurgical patients.

The peptide adrenomedullin (ADM), comprising 52 amino acids, was originally isolated from a human phenochromocytome (Kitamura K et al. 1993). ADM has been shown to have hypotensive, immune modulating, metabolic and vascular actions. It is a potent vasodilator, and its widespread production in tissues helps to maintain blood supply to individual organs. ADM stabilises the microcirculation and protect against endothelial permeability and consequent organ failure and has shown considerable promise, especially in the fields of sepsis (Andaluz-Ojeda et al. 2015), lower respiratory tract infections (Hartmann et al. 2012, Albrich et al. 2011a, Albrich et al. 2011b, Albrich et al. 2013), lung transplantation (Riera et al. 2015) and thoracic surgery (Schoe et al. 2015).

Endothelin-1 is a member of a family of 21-amino acid peptides and is considered having an important pathophysiological role in cardiovascular dysfunction (Haaf et al. 2014, Papssoutiriou et al. 2006). ET-1 is a potent vasoconstrictor agent, synthesized mainly by endothelial cells. It exerts mitogenic effects on smooth muscle cells and has a role in angiogenesis and development of atherosclerosis by causing endothelial dysfunction and inflammation (Haaf et al. 2014, Zhang et al. 2017). The plasma levels of pro ET-1 have been shown to be elevated in several cardiovascular pathologies such as acute myocardial infarction, congestive heart failure, sepsis induced myocardial dysfunction, pulmonary hypertension, coronary artery disease, hypertension, aging, diabetes as well as ischemic cardiac diseases and infarctions (Haaf et al. 2014, Zhang et al. 2017, Setiano et al. 2016, Lundberg 2016).

PCT is the prehormone of calcitonin, which is normally secreted by the C cells of the thyroid in response to hypercalcemia; under these normal conditions, negligible serum PCT concentrations are detected (Whicher et al. 2001). In case of a bacterial infection, inflammation or sepsis procalcitonin levels are elevated and PCT is a well-known and routinely used biomarker for the diagnosis of these diseases.

Although ADM, proET-1 and PCT are currently employed in different clinical scenarios, the biological function of the biomarkers exhibits common features. In particular, ADM and PCT have both been shown to interact with calcitonin receptor complexes in endothelial cells, in particular the calcitonin gene-related peptide receptor (CGRP receptor) (Sexton et al. 2008, Nikitenko et al. 2006, Smith et al. 2002). Furthermore, also the plasma levels of proET-1 have been shown to correlate with CGRP indicating a common signaling path for instance in the regulation of blood pressure (Parlapiano et al. 1999; Wang et al. 2004).

Calcitonin gene-related peptide (CGRP) is an endogenous vasoactive peptide encoded by the calcitonin gene in nerve cells and has been shown to be distributed throughout the cardiovascular system and to act as a potent vasodilator. CGRP and its receptors have in particular been proposed to be involved in migraine pathophysiology (Ho Tony W. et al. GRP and its receptors provide new insights into migraine pathophysiology Nature Reviews Neurology volume 6, pages 573-582 (2010)). Plasma levels of CGRP also have however also been shown to be elevated in sepsis (Joyce et al. 1990) and for CGRP receptors a more general role in the control of pain and inflammation has been proposed (Benemei et al. 2009)

Without intending to be bound by theory, the surprising finding of a common potential of proADM, PCT and proET-1 in the diagnosis and prognosis of postsurgical adverse events as demonstrated in the data may relate to their common interaction with Calcitonin gene-related peptide (CGRP) and their receptors as well as other receptors of the calcitonin complex receptor family.

Also, the method can be valuable as predictor for the risk of an adverse event and in guiding the monitoring of postsurgical patients. When the level of the biomarkers proADM, PCT and/or proET-1 indicates an elevated likelihood of a postsurgical adverse event, the medical personal may adjust the frequency and/or intensity of the monitoring. Depending on the prognosis of the adverse events, specialized diagnostic tools may be employed, and routine examination proceeding conducted at more frequent intervals.

In this regard the method is particularly valuable for risk assessment or stratification and allows for a grouping or classifying of patients into different groups, such as risk groups, requiring more frequent monitoring or additional diagnosis, or therapy groups that receive certain differential therapeutic measures depending on their classification.

The potential of an early diagnosis or prognosis using the biomarkers described herein thus not only allows for a prompt therapy guidance for an improved patient outcome, but also aids in employing resource-efficient strategies.

In one embodiment the adverse event is a cardiovascular or cerebrovascular event.

In one embodiment the adverse event is a myocardial infarction (MI).

In one embodiment the adverse event is a myocardial injury after non-cardiac surgery (MINS).

In one embodiment the adverse event is a perioperative myocardial injury (PMI).

In one embodiment the adverse event is a major adverse cardiovascular or cerebrovascular event (MACCE).

In one embodiment the adverse event is a stroke and/or a transient ischemic attack.

In one embodiment the adverse event is an infection.

In one embodiment the adverse event is a fungal, a bacterial or a viral infection.

In one embodiment the adverse event is a local infection as for example a respiratory tract infection, a urinary tract infection, a skin infection or an abdominal infection.

In one embodiment the adverse event is a systemic blood infection, sepsis, severe sepsis and/or septic shock.

In one embodiment the adverse event is a cardiovascular or cerebrovascular event and an infection, occurring preferably in close temporal relation to each other, preferably occurring simultaneously.

In one embodiment the adverse event is a myocardial injury after non-cardiac surgery (MINS) and an infection.

In one embodiment the adverse event is perioperative myocardial injury (PMI) and an infection.

In one embodiment the adverse event is a major cardiovascular or cerebrovascular event (MACCE) and an infection.

In one embodiment the adverse event is a cardiovascular or cerebrovascular event and a blood infection.

In one embodiment the adverse event is a myocardial injury after non-cardiac surgery (MINS) and a blood infection.

In one embodiment the adverse event is a major cardiovascular or cerebrovascular event (MACCE) and a blood infection.

In one embodiment the adverse event is perioperative myocardial injury (PMI) and a blood infection.

It is entirely surprising that based upon the measurement of the biomarkers described herein an accurate and reliable conclusion can be made as to whether the patient suffers postsurgically a cardiovascular or cerebrovascular event, such as MINS or MACCE, and an infection, such as a blood infection. This prognostic ability of proADM, PCT and/or pro ET-1 in the particular settings described herein is novel and surprising, and enables improvements in the detection, prediction and management of adverse events surrounding operations. The combined assessment of these possible adverse events is also of particularly high clinical relevance. The optimal therapy guidance may crucially depend on such an assessment since measures or medication for treating an infection may be unfavorable for the cardiovascular or cerebrovascular event or vice versa. By providing a diagnosis or prognosis for both adverse events the clinical personnel may choose the best possible therapeutic approach.

When predicting or diagnosing e.g. a MACCE, PMI or a MINS the subsequent clinical procedures such as a bypass surgery or an angioplasty elevate the risk of the deterioration of a possible infection. An early preemptive anti-infective therapy, such as an antibiotic therapy, may be of critical importance for an overall positive patient outcome.

In one embodiment the surgery is a non-cardiac surgery and at the time when the sample is isolated from the patient, the patient has undergone, is undergoing or will undergo a non-cardiac surgery. Preferably the non-cardiac surgery is a major non-cardiac surgery that includes a more extensive surgery and typically has to be performed under general anesthesia and requires hospitalization with at least one overnight stay.

In one embodiment the surgery is an abdominal or gastrointestinal surgery and at the time when the sample is isolated from the patient, the patient has undergone, is undergoing or will undergo an abdominal or gastrointestinal surgery.

The term hospitalization refers preferably to a maintained control and/or assessment of the medical condition of a patient in a hospital, whether it be in an emergency room, ward, intensive care station, or other area of a hospital or clinic.

In one embodiment the surgery is an abdominal surgery and at the time when the sample is isolated of the patient, when the patient has undergone, is undergoing or will undergo abdominal surgery.

In one embodiment the patient is 50 years or older, or 55, 60, 65, 70 or 75 years or older. Patients in these age groups are at a particularly high risk of developing an adverse event after surgery, rendering the method described herein particularly valuable for said patient group.

In preferred embodiments of the method of the present invention said sample is isolated from said patient about 30 minutes, 1 hour, 2 hours, 3 hours, 4, hours, 5 hours, 6 hours, 7 hours, 8 hours, 9 hours, 10 hours, 11 hours, 12 hours, 14 hours, 16 hours, 18 hours, 20 hours 22 hours, 24 hours, 30 hours, 36 hours, 42 hours, 48 hours, 60 hours, 72 hours, 84 hours, 4 days, 5 days, 6 days, 7 days, 8 days 9 days or 10 days before or after surgery.

In other embodiments, the sample is isolated at time points of 30 minutes to 12 hours, 12-36 hours, 3-5 days, 7-14 days, 8-12 days, or 9-11 days before or after surgery.

Ranges between any given of the above values may be employed to define the time point of obtaining the sample.

In one embodiment the sample is isolated preoperatively within 7 days, preferably within 3 days, one day 12 hours or less before the surgery.

In one embodiment the sample is isolated preoperatively at least 3 hours, preferably at least 6 hours before the surgery.

In one embodiment the sample is isolated preoperatively within 7 days, preferably within 3 days, more preferably within one day and at least 3 hours, preferably at least 6 hours before the surgery.

The embodiments above allow for a prediction of a postsurgical adverse before the surgery has started. In cases where a particularly high risk of an adverse event is established, the surgery may be postponed or even cancelled. Furthermore, a preoperative risk assessment of postsurgical adverse event allows for the administration of preventive medicine, e.g. antibiotics in case of infection or blood thinning medication in case of cardiovascular or cerebrovascular events.

In one embodiment the sample is isolated from the patients postsurgically at least 12 hours, 24 hours, 48 hours, 72 hours or more after surgery, and/or within 72 hours, 48 hours, 24 hours, 12 hours or less after the surgery.

In one embodiment the sample is isolated postsurgically within 12 hours, preferably within 6 hours after surgery. The embodiment relates preferably to a sample isolation, while the patient is monitored on the post anesthetic care unit (PACU)

In one embodiment the sample is isolated one day after surgery (POD1). In this case the sample is preferably isolated in between 12 hours and 36 hours after surgery, preferably in between 18 hours and 30 hours after surgery.

In one embodiment the sample is isolated two days after surgery (POD2). In this case the sample is preferably isolated in between 36 hours and 60 hours after surgery, preferably in between 42 hours and 54 hours after surgery.

In one embodiment the sample is isolated three days after surgery (POD3). In this case the sample is preferably isolated between 60 hours and 84 hours after surgery, preferably in between 66 hours and 78 hours after surgery.

In some embodiments, the invention describes the use of the levels of the biomarkers from a sample at for example 1, 2 or 3 time points, such as at preoperatively, on the post anesthesia care unit (PACU) or one, two or three days after surgery (POD1 through POD3) in assessing the likelihood of a postsurgical adverse event. As the data below show the different temporal development of the level of the biomarkers in patients with and without an adverse event allow for a particularly reliable prognosis or diagnosis, when taking samples at two time points or more and analyzing the ratio or absolute difference for the different sample points.

In one embodiment a first sample is isolated presurgically and/or perioperatively and a second sample is isolated postsurgically.

In one embodiment a first sample is isolated presurgically and a second sample is isolated perioperatively and/or postsurgically.

In one embodiment a first sample is isolated within 3 days, preferably within one day or less before the surgery and a second sample is isolated within 12 hours, preferably within 6 hours or less after surgery.

In one embodiment a first sample is isolated within 3 days, preferably within one day or less before the surgery and a second sample is isolated within one day, two days, three days or more after surgery.

In one embodiment a first sample isolated within 12 hours, preferably within 6 hours after surgery and a second sample is isolated within one day, two days, three days or more after surgery.

In one embodiment a first sample is isolated one day after surgery and a second sample is isolated two days, three days or more after surgery.

In one embodiment a first sample isolated two days after surgery and a second sample is isolated three days or more after surgery.

In one embodiment a first sample is isolated from the patient at a first time point and a second sample is isolated from the patient at a second time point, wherein preferably in between the first and second time point at least 6 hours, 12 hours, 24 hours, 36 hours, 48 hours or more have passed. It is preferred however that less than 72 hours, 48 hours or less have passed such that the preferred time span in between the sample isolation may be for instance in between 6 hours and 72 hours, 6 hours and 48 hours, 12 and 72 hours, 12 and 48 hours or any other combination of the above mentioned preferred maximal and minimal time spans.

In the context of the present invention, determining a lower level of a marker in a second sample as compared to a first sample can be indicative of decreasing levels of the respective marker in the patient over the time of observation. Conversely, elevated levels in a second sample as compared to a first sample might indicate increasing levels of the marker over the time of observation.

In one embodiment the method for the diagnosis, prognosis, risk assessment and/or risk stratification of an adverse event in the health of a postsurgical patient, comprises the isolation of a first and a second sample at a first and a second time point, wherein the likelihood of an adverse event correlates with the absolute difference of the level of the biomarker, the ratio of the level of the biomarker and/or the rate of change of the level of the biomarker in regards to said first and second time point.

The rate of change of the level of the biomarker in regard to said first and second time point preferably refers to the absolute difference in the level of the biomarker over the temporal difference between said first and second time point.

It is to be understood that in regards to first and second time point any combination of the herein disclosed time points for isolating a sample may be particularly preferred without intending to be limiting the first time and second points may thus preferably refer to a time point within 3 days, within 1 day or less before surgery, within 6 hours, within 12 hours or less after surgery as well as one day, two days or three days after surgery, wherein between the first and second time point preferably at least 6 hours, 12 hours, 24 hours or more have passed.

In one embodiment a first sample is isolated from the patient postsurgically at a first time point and a second sample is isolated from the patient postsurgically at a subsequent second time point, wherein preferably between the first and second time point at least 12 hours, 24 hours, 36 hours, 48 hours or more have passed and an increase or a leveling in the level of proADM, PCT and/or proET-1 or fragment(s) thereof indicates an elevated likelihood of a postsurgical adverse event.

In preferred embodiments of the invention, the sample is selected from the group consisting of a blood sample, such as a whole blood sample, a serum sample or a plasma sample, a saliva sample and a urine sample.

In one embodiment, determining a level of proADM or fragment(s) thereof comprises determining a level of MR-proADM in the sample. As demonstrated in the examples below, MR-proADM, preferably determined using established immunoassay products of B.R.A.H.M.S (Hennigsdorf, Germany), shows a reliable and effective prognosis according to the methods described herein. Alternative ADM molecules such as the mature adrenomedullin or PAMP, including precursors or fragments, may also be employed.

In one embodiment, determining a level of PCT or fragment(s) thereof comprises determining a level of PCT 1-116, PCT 2-116 or PCT 3-116 in the sample. As demonstrated in the examples below, PCT 1-116, PCT 2-116 or PCT 3-116, are preferably determined using established immunoassay products of B.R.A.H.M.S (Hennigsdorf, Germany), shows a reliable and effective prognosis according to the methods described herein. Alternative PCT molecules such as calcitonin or katacalcin, including precursors or fragments, may also be employed.

In one embodiment, determining a level of proET-1 or fragment(s) thereof comprises determining a level of CT-proET-1 in the sample. As demonstrated in the examples below, CT-proET-1, preferably determined using established immunoassay products of B.R.A.H.M.S (Hennigsdorf, Germany), shows a reliable and effective prognosis according to the methods described herein. Alternative proET-1 molecules, including precursors or fragments, may also be employed.

In one embodiment the method additionally comprises a therapy guidance, stratification and/or control. The method allows to base the application of certain therapies, therapeutic actions or medical interventions on the determined level of one or more biomarkers in order to treat or prevent the adverse event. The therapy guidance, stratification and/or control will depend on the adverse event, in particular in regard to its type and/or diagnosed/predicted severity.

In one embodiment the level of the at least one biomarker correlates with the likelihood of an infection that is indicative of initiation of a treatment with an anti-infective agent, preferably an antimicrobial or antibiotic agent.

In some embodiments the method additionally comprises a molecular analysis of a sample from said patient for detecting the infection. The sample used for the molecular analysis for detecting an infection preferably is a blood sample, blood culture sample or tissue sample. In a preferred embodiment the molecular analysis is a method aiming to detect one or more biomolecules derived from a pathogen that may cause said infection. Said one or more biomolecule may be a nucleic acid, protein, sugar, carbohydrates, lipid and or a combination thereof such as glycosylated protein, preferably a nucleic acid. Said biomolecule preferably is specific for one or more pathogen(s). According to preferred embodiments, such biomolecules are detected by one or more methods for analysis of biomolecules selected from the group comprising nucleic acid amplification methods such as PCR, qPCR, RT-PCR, qRT-PCR or isothermal amplification, mass spectrometry, detection of enzymatic activity and immunoassay-based detection methods. Further methods of molecular analysis are known to the person skilled in the art and are comprised by the method of the present invention.

Using the additional molecular analysis of the sample a person skilled in the art will also be able to select the appropriate anti-infective agent to treat the infection. Advantageously, the level of the determined the biomarker may aid in guiding the intensity of treatment. Generally speaking, a higher level of the biomarker may indicate a more severe state of the infection and thus guide a more intense, e.g. more frequent or higher dosed treatment.

In one embodiment the level of the at least one biomarker correlates with the likelihood of an adverse cardiovascular or cerebrovascular event that is indicative of further diagnostic assessment selected from the group consisting of a CT scan, an MRI scan, an angiogram, a arteriography, a X-Ray, an Echocardiogram, an ECG and a EEG.

After assessing the likelihood of an adverse cardiovascular or cerebrovascular event additional testing may be performed, e.g., to gain more information on the patient's condition. For example, in the case of a predicted or diagnosed myocardial infarction or myocardial injury, the patient may be sent for an electrocardiogram (preferably a 12-lead ECG); more extensive imaging studies including coronary angiography or corony catheterization.

In case of a predicted or diagnosed stroke, the patient may be sent for a neurological examination (such as the National Institutes of Health Stroke Scale (NIHSS), a CT scan, an MRI scan, a Doppler ultrasound or an arteriography.

In this regard the potential of the biomarkers for an early diagnosis or predication is particularly valuable, since the deterioration of the patients overall health critical depends on a prompt counter therapy.

In some embodiments, the methods may include the use of additional diagnostic methods to identify the underlying pathology. Any diagnostic methods known in the art can be used, and one of skill in the art will be able to select diagnostic methods that are appropriate for cardiovascular or cerebrovascular events. In some embodiments, the methods described herein include other diagnostic methods in addition to or as an alternative to the measurement of other biomarkers, e.g., physical measurements of cardiac function or motoric function as are known in the art. The further diagnostic means may also include the determination of other biomarkers indicative of cardiac disease such as cardiac troponin (cTn).

In one embodiment the level of the at least one biomarker correlates with the likelihood of an adverse cardiovascular or cerebrovascular event that is indicative of an initiation or preparation of an anticoagulation therapy, an oxygen therapy, a lysis therapy, a percutaneous coronary intervention, a percutaneous transluminal angioplasty, a coronary artery bypass graft and/or a stent implantation.

In some embodiments a medical treatment comprises the administration of statins, β-blockers, α2-adrenergic agonists or other anti-ischemic agents such calcium-channel blockers or nitrates and may be employed as preventive therapeutic strategy before an adverse event such as a myocardial infarction or injury occurs.

In one embodiment the level of the at least one biomarker correlates with the likelihood of a postsurgical adverse event that is indicative of the patient requiring frequent monitoring and/or critical care.

Preferably, critical care relates to therapeutic measures that aim at stabilizing or ameliorating the health condition of a patient that is at risk of developing or is in the state of a life-threatening condition. For instance, in case of a severe infection, e.g. a blood infection examples of critical care treatments include but are not limited to fluid therapy, intravenous antibiotics, in some embodiments essentially any treatment beyond oral antibiotics. In regard to a cardiovascular or cerebrovascular event, critical care may for instance relate to oxygen therapy, a lysis therapy or an emergency or coronary artery bypass graft and/or a stent implantation.

Frequent monitoring preferably refers to a frequent assessment of the health state of the patient in regard to the adverse event and may comprise for instance the monitoring of general parameters for the health state such as blood pressure, respiratory rate, partial pressure of oxygen, temperature etc. or more specific further parameters or diagnostic means as described above in regard to the adverse event. The frequent monitoring may preferably refer to a monitoring with an intermittent time interval of at most 6 hours, preferably 3 hours, 2 hours or 1 hour.

In some embodiments, e.g. in the ICU, monitoring is continuous.

In some embodiments a level of the at least one biomarker above a threshold indicates an elevated likelihood of an adverse event.

In some embodiments a level of the at least one biomarker above a threshold indicates a decreased likelihood of an adverse event.

According to the present invention, the term “indicate” in the context of “indicative of an elevated likelihood of adverse event” and “indicative of a decreased likelihood of adverse event” is intended as a measure of risk and/or likelihood. Preferably, the “indication” of the presence or absence of an adverse event or the presence or absence of a risk for developing said adverse event is intended as a risk assessment or a likelihood for the diagnosis, and is typically not to be construed in a limiting fashion as to point definitively to the absolute presence or absence of said event.

Therefore, the term “indicative of an adverse event” can be understood as indicating a high likelihood of the occurrence of an adverse event, respectively

Keeping the above in mind, using the threshold values disclosed herein, allows for reliable diagnosis of adverse events or an estimation of their risk and enables appropriate action by a medical professional.

It is further to be understood that in embodiments of the invention, deviations from the disclosed possible thresholds below are also disclosed and claimed, such as deviations of ±20%, 19%, 18%, 17%, 16%, 15%, 14%, 13%, 12%, 11%, 10%, 9%, 8%, 7%, 6%, 5%, 4%, 3%, 2% or 1% as well as the exact threshold.

The threshold values disclosed herein refer preferably to measurements of the protein level of proADM, PCT, proET-1 or fragments thereof in a plasma sample obtained from a patient by means of the Thermo Scientific BRAHMS KRYPTOR assay or an assay suitable for an automated system. Accordingly, the values disclosed herein may vary to some extent depending on the detection/measurement method employed, and the specific values disclosed herein are intended to also read on the corresponding values determined by other methods.

All threshold values disclosed herein relating to the level of a marker or biomarker, such as ADM, PCT or proET-1, are to be understood as “equal or above” a certain threshold or “equal or below” a certain threshold. For example, an embodiment relating to a level of proADM or fragment(s) thereof above 1.2 nmol/L is to be understood as relating to a level of proADM or fragment(s) thereof equal or above 1.2 nmol/L.

### Embodiments Relating to Determining a Level of proADM or Fragment(s) Thereof for Diagnosing or Predicting a Cardiovascular or Cerebrovascular Event

### ADM/MACCE:

In one embodiment determining a level of at least one biomarker comprises determining the level of proADM or fragment(s) thereof and a level of proADM or fragment(s) thereof above a threshold value indicates an elevated likelihood of an adverse cardiovascular or cerebrovascular event, preferably a MACCE.

In one embodiment the sample is isolated preoperatively and determining a level of at least one biomarker comprises determining the level of proADM or fragment(s) thereof and a level of proADM or fragment(s) thereof above a threshold value±20% or more selected from a range of values in between 0.7 nmol/L and 0.8 nmol/L indicates an elevated likelihood of an adverse cardiovascular or cerebrovascular event, preferably a MACCE. Any value within this range may be considered as an appropriate threshold value. For example, 0.70, 0.71, 0.72, 0.73, 0.74, 0.75, 0.76, 0.77, 0.78, 0.79, 0.8 nmol/L.

In one embodiment the sample is isolated postsurgically within 12 hours, preferably within 6 hours after surgery and determining a level of at least one biomarker comprises determining the level of proADM or fragment(s) thereof and a level of proADM or fragment(s) thereof above a threshold value±20% or more selected from a range of values in between 1.1 nmol/L and 1.4 nmol/L indicates an elevated likelihood of an adverse cardiovascular or cerebrovascular event, preferably a MACCE. Any value within this range may be considered as an appropriate threshold value. For example, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.29, 1.3, 1.31, 1.32, 1.33, 1.34, 1.35, 1.36, 1.37, 1.38, 1.39, 1.4 nmol/L.

In one embodiment the sample is isolated postsurgically one day after surgery and determining a level of at least one biomarker comprises determining the level of proADM or fragment(s) thereof and a level of proADM or fragment(s) thereof above a threshold value±20% or more selected from a range of values in between 1.4 nmol/L and 1.8 nmol/L indicates an elevated likelihood of an adverse cardiovascular or cerebrovascular event, preferably a MACCE. Any value within this range may be considered as an appropriate threshold value. For example, 1.4, 1.41, 1.42, 1.43, 1.44, 1.45, 1.46, 1.47, 1.48, 1.49, 1.5, 1.51, 1.52, 1.53, 1.54, 1.55, 1.56, 1.57, 1.58, 1.59, 1.6, 1.61, 1.62, 1.63, 1.64, 1.65, 1.66, 1.67, 1.68, 1.69, 1.7, 1.71, 1.72, 1.73, 1.74, 1.75, 1.76, 1.77, 1.78, 1.79, 1.8 nmol/L.

In one embodiment the sample is isolated postsurgically two days after surgery and determining a level of at least one biomarker comprises determining the level of proADM or fragment(s) thereof and a level of proADM or fragment(s) thereof above a threshold value±20% or more selected from a range of values in between 1.3 nmol/L and 1.8 nmol/L indicates an elevated likelihood of an adverse cardiovascular or cerebrovascular event, preferably a MACCE. Any value within this range may be considered as an appropriate threshold value. For example, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5, 1.51, 1.52, 1.53, 1.54, 1.55, 1.56, 1.57, 1.58, 1.59, 1.6, 1.61, 1.62, 1.63, 1.64, 1.65, 1.66, 1.67, 1.68, 1.69, 1.7, 1.71, 1.72, 1.73, 1.74, 1.75, 1.76, 1.77, 1.78, 1.79, 1.8 nmol/L.

In one embodiment the sample is isolated postsurgically three days after surgery and determining a level of at least one biomarker comprises determining the level of proADM or fragment(s) thereof and a level of proADM or fragment(s) thereof above a threshold value±20% or more selected from a range of values in between 1.3 nmol/L and 1.9 nmol/L indicates an elevated likelihood of an adverse cardiovascular or cerebrovascular event, preferably a MACCE. Any value within this range may be considered as an appropriate threshold value. For example, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5, 1.51, 1.52, 1.53, 1.54, 1.55, 1.56, 1.57, 1.58, 1.59, 1.6, 1.61, 1.62, 1.63, 1.64, 1.65, 1.66, 1.67, 1.68, 1.69, 1.7, 1.71, 1.72, 1.73, 1.74, 1.75, 1.76, 1.77, 1.78, 1.79, 1.8, 1.81, 1.82, 1.83, 1.84, 1.85, 1.86, 1.87, 1.88, 1.89, 1.9 nmol/L.

In one embodiment the sample isolated postsurgically and determining a level of at least one biomarker comprises determining the level of proADM or fragment(s) thereof and a level of proADM or fragment(s) thereof above a threshold value±20% or more selected from a range of values in between 1.1 nmol/L and 1.5 nmol/L indicates an elevated likelihood of an adverse cardiovascular or cerebrovascular event, preferably a MACCE. Any value within this range may be considered as an appropriate threshold value. For example, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.29, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5 nmol/L.

In one embodiment the sample isolated postsurgically at one day, two days, three days or more after surgery and determining a level of at least one biomarker comprises determining the level of proADM or fragment(s) thereof and a level of proADM or fragment(s) thereof above a threshold value±20% or more selected from a range of values in between 1.4 nmol/L and 1.8 nmol/L indicates an elevated likelihood of an adverse cardiovascular or cerebrovascular event, preferably a MACCE. Any value within this range may be considered as an appropriate threshold value. For example, 1.4, 1.41, 1.42, 1.43, 1.44, 1.45, 1.46, 1.47, 1.48, 1.49, 1.5, 1.51, 1.52, 1.53, 1.54, 1.55, 1.56, 1.57, 1.58, 1.59, 1.6, 1.61, 1.62, 1.63, 1.64, 1.65, 1.66, 1.67, 1.68, 1.69, 1.7, 1.71, 1.72, 1.73, 1.74, 1.75, 1.76, 1.77, 1.78, 1.79, 1.8 nmol/L.

In one embodiment a first sample is isolated from the patient postsurgically at a first time point, preferably one day or more after surgery, and a second sample is isolated from the patient postsurgically at a subsequent second time point, wherein preferably between the first and second time point at least 12 hours, 24 hours, 36 hours, 48 hours or more have passed and determining a level of at least one biomarker comprises determining the level of proADM or fragment(s) thereof and an increase in the level of proADM or fragment(s) thereof indicates an elevated likelihood of an adverse cardiovascular or cerebrovascular event, preferably a MACCE.

In one embodiment a first sample is isolated from the patient postsurgically at a first time point, preferably one day or more after surgery, and a second sample is isolated from the patient postsurgically at a subsequent second time point, wherein preferably between the first and the second time point at least 12 hours, 24 hours, 36 hours, 48 hours or more have passed and determining a level of at least one biomarker comprises determining the level of proADM or fragment(s) thereof and an increase of the level of proADM or fragment(s) thereof above a threshold value±20% or more selected from a range of values in between 0.01 nmol/L and 0.5 nmol/L indicates an elevated likelihood of an adverse cardiovascular or cerebrovascular event, preferably a MACCE. Any value within this range may be considered as an appropriate threshold value for the increase of proADM or fragment(s) in between the first and second time point. For example, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5 nmol/L.

### ADM/MINS:

In one embodiment determining a level of at least one biomarker comprises determining the level of proADM or fragment(s) thereof and a level of proADM or fragment(s) thereof above a threshold value indicates an elevated likelihood of an adverse cardiovascular or cerebrovascular event, preferably a MINS.

In one embodiment the sample is isolated preoperatively and determining a level of at least one biomarker comprises determining the level of proADM or fragment(s) thereof and a level of proADM or fragment(s) thereof above a threshold value±20% or more selected from a range of values in between 0.7 nmol/L and 0.8 nmol/L indicates an elevated likelihood of an adverse cardiovascular or cerebrovascular event, preferably a MINS. Any value within this range may be considered as an appropriate threshold value. For example, 0.70, 0.71, 0.72, 0.73, 0.74, 0.75, 0.76, 0.77, 0.78, 0.79, 0.8 nmol/L.

In one embodiment the sample is isolated postsurgically within 12 hours, preferably within 6 hours after surgery and determining a level of at least one biomarker comprises determining the level of proADM or fragment(s) thereof and a level of proADM or fragment(s) thereof above a threshold value±20% or more selected from a range of values in between 1.1 nmol/L and 1.4 nmol/L indicates an elevated likelihood of an adverse cardiovascular or cerebrovascular event, preferably a MINS. Any value within this range may be considered as an appropriate threshold value. For example, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.29, 1.3, 1.31, 1.32, 1.33, 1.34, 1.35, 1.36, 1.37, 1.38, 1.39, 1.4 nmol/L.

In one embodiment the sample is isolated postsurgically one day after surgery and determining a level of at least one biomarker comprises determining the level of proADM or fragment(s) thereof and a level of proADM or fragment(s) thereof above a threshold value±20% or more selected from a range of values in between 1.3 nmol/L and 1.8 nmol/L indicates an elevated likelihood of an adverse cardiovascular or cerebrovascular event, preferably a MINS. Any value within this range may be considered as an appropriate threshold value. For example, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5, 1.51, 1.52, 1.53, 1.54, 1.55, 1.56, 1.57, 1.58, 1.59, 1.6, 1.61, 1.62, 1.63, 1.64, 1.65, 1.66, 1.67, 1.68, 1.69, 1.7, 1.71, 1.72, 1.73, 1.74, 1.75, 1.76, 1.77, 1.78, 1.79, 1.8 nmol/L.

In one embodiment the sample is isolated postsurgically two days after surgery and determining a level of at least one biomarker comprises determining the level of proADM or fragment(s) thereof and a level of proADM or fragment(s) thereof above a threshold value±20% or more selected from a range of values in between 1.3 nmol/L and 1.8 nmol/L indicates an elevated likelihood of an adverse cardiovascular or cerebrovascular event, preferably a MINS. Any value within this range may be considered as an appropriate threshold value. For example, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5, 1.51, 1.52, 1.53, 1.54, 1.55, 1.56, 1.57, 1.58, 1.59, 1.6, 1.61, 1.62, 1.63, 1.64, 1.65, 1.66, 1.67, 1.68, 1.69, 1.7, 1.71, 1.72, 1.73, 1.74, 1.75, 1.76, 1.77, 1.78, 1.79, 1.8 nmol/L.

In one embodiment the sample is isolated postsurgically three days after surgery and determining a level of at least one biomarker comprises determining the level of proADM or fragment(s) thereof and a level of proADM or fragment(s) thereof above a threshold value±20% or more selected from a range of values in between 1.3 nmol/L and 1.8 nmol/L indicates an elevated likelihood of an adverse cardiovascular or cerebrovascular event, preferably a MINS. Any value within this range may be considered as an appropriate threshold value. For example, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5, 1.51, 1.52, 1.53, 1.54, 1.55, 1.56, 1.57, 1.58, 1.59, 1.6, 1.61, 1.62, 1.63, 1.64, 1.65, 1.66, 1.67, 1.68, 1.69, 1.7, 1.71, 1.72, 1.73, 1.74, 1.75, 1.76, 1.77, 1.78, 1.79, 1.8 nmol/L.

In one embodiment the sample isolated postsurgically and determining a level of at least one biomarker comprises determining the level of proADM or fragment(s) thereof and a level of proADM or fragment(s) thereof above a threshold value±20% or more selected from a range of values in between 1.1 nmol/L and 1.5 nmol/L indicates an elevated likelihood of an adverse cardiovascular or cerebrovascular event, preferably a MINS. Any value within this range may be considered as an appropriate threshold value. For example, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.29, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5 nmol/L.

In one embodiment the sample isolated postsurgically at one day, two days, three days or more after surgery and determining a level of at least one biomarker comprises determining the level of proADM or fragment(s) thereof and a level of proADM or fragment(s) thereof above a threshold value±20% or more selected from a range of values in between 1.3 nmol/L and 1.8 nmol/L indicates an elevated likelihood of an adverse cardiovascular or cerebrovascular event, preferably a MINS. Any value within this range may be considered as an appropriate threshold value. For example, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5, 1.51, 1.52, 1.53, 1.54, 1.55, 1.56, 1.57, 1.58, 1.59, 1.6, 1.61, 1.62, 1.63, 1.64, 1.65, 1.66, 1.67, 1.68, 1.69, 1.7, 1.71, 1.72, 1.73, 1.74, 1.75, 1.76, 1.77, 1.78, 1.79, 1.8 nmol/L.

In one embodiment a first sample is isolated from the patient postsurgically at a first time point, preferably one day or more after surgery, and a second sample is isolated from the patient postsurgically at a subsequent second time point, wherein preferably between the first and second time point at least 12 hours, 24 hours, 36 hours, 48 hours or more have passed and determining a level of at least one biomarker comprises determining the level of proADM or fragment(s) thereof and an increase or a leveling in the level of proADM or fragment(s) thereof indicates an elevated likelihood of an adverse cardiovascular or cerebrovascular event, preferably a MINS.

In one embodiment a first sample is isolated from the patient postsurgically at a first time point, preferably one day or more after surgery, and a second sample is isolated from the patient postsurgically at a subsequent second time point, wherein preferably between the first and the second time point at least 12 hours, 24 hours, 36 hours, 48 hours or more have passed and determining a level of at least one biomarker comprises determining the level of proADM or fragment(s) thereof and an increase of the level of proADM or fragment(s) thereof above a threshold value±20% or more selected from a range of values in between 0.01 nmol/L and 0.5 nmol/L indicates an elevated likelihood of an adverse cardiovascular or cerebrovascular event, preferably a MINS. Any value within this range may be considered as an appropriate threshold value for the increase of proADM or fragment(s) in between the first and second time point. For example, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5 nmol/L.

### Embodiments Relating to Determining a Level of PCT or Fragment(s) Thereof for Diagnosing or Predicting a Cardiovascular or Cerebrovascular Event

### PCT/MACCE:

In one embodiment determining a level of at least one biomarker comprises determining the level of PCT or fragment(s) thereof and a level of PCT or fragment(s) thereof above a threshold value indicates an elevated likelihood of an adverse cardiovascular or cerebrovascular event, preferably a MACCE.

In one embodiment the sample is isolated postsurgically within 12 hours, preferably within 6 hours after surgery and determining a level of at least one biomarker comprises determining the level of PCT or fragment(s) thereof and a level of PCT or fragment(s) thereof above a threshold value±20% or more selected from a range of values in between 0.1 μg/L and 0.4 μg/L indicates an elevated likelihood of an adverse cardiovascular or cerebrovascular event, preferably a MACCE. Any value within this range may be considered as an appropriate threshold value. For example 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4 μg/L.

In one embodiment the sample is isolated postsurgically one day after surgery and determining a level of at least one biomarker comprises determining the level of PCT or fragment(s) thereof and a level of PCT or fragment(s) thereof above a threshold value±20% or more selected from a range of values in between 0.6 μg/L and 1.2 μg/L indicates an elevated likelihood of an adverse cardiovascular or cerebrovascular event, preferably a MACCE. Any value within this range may be considered as an appropriate threshold value. For example, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, 1.2 μg/L.

In one embodiment the sample is isolated postsurgically two days after surgery and determining a level of at least one biomarker comprises determining the level of PCT or fragment(s) thereof and a level of PCT or fragment(s) thereof above a threshold value±20% or more selected from a range of values in between 0.8 μg/L and 1.8 μg/L indicates an elevated likelihood of an adverse cardiovascular or cerebrovascular event, preferably a MACCE. Any value within this range may be considered as an appropriate threshold value. For example, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.29, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5, 1.51, 1.52, 1.53, 1.54, 1.55, 1.56, 1.57, 1.58, 1.59, 1.6, 1.61, 1.62, 1.63, 1.64, 1.65, 1.66, 1.67, 1.68, 1.69, 1.7, 1.71, 1.72, 1.73, 1.74, 1.75, 1.76, 1.77, 1.78, 1.79, 1.8 μg/L.

In one embodiment the sample is isolated postsurgically three days after surgery and determining a level of at least one biomarker comprises determining the level of PCT or fragment(s) thereof and a level of PCT or fragment(s) thereof above a threshold value±20% or more selected from a range of values in between 1 μg/L and 2 μg/L indicates an elevated likelihood of an adverse cardiovascular or cerebrovascular event, preferably a MACCE. Any value within this range may be considered as an appropriate threshold value. For example, 1, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.29, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5, 1.51, 1.52, 1.53, 1.54, 1.55, 1.56, 1.57, 1.58, 1.59, 1.6, 1.61, 1.62, 1.63, 1.64, 1.65, 1.66, 1.67, 1.68, 1.69, 1.7, 1.71, 1.72, 1.73, 1.74, 1.75, 1.76, 1.77, 1.78, 1.79, 1.8, 1.81, 1.82, 1.83, 1.84, 1.85, 1.86, 1.87, 1.88, 1.89, 1.9, 1.91, 1.92, 1.93, 1.94, 1.95, 1.96, 1.97, 1.98, 1.99, 2 μg/L.

In one embodiment the sample is isolated postsurgically at one day or more after surgery and determining a level of at least one biomarker comprises determining the level of PCT or fragment(s) thereof and a level of PCT or fragment(s) thereof above a threshold value±20% or more selected from a range of values in between 0.9 μg/L and 1.5 μg/L indicates an elevated likelihood of an adverse cardiovascular or cerebrovascular event, preferably a MACCE. Any value within this range may be considered as an appropriate threshold value. For example, 0.9, 0.91, 0.92, 0.93, 0.94, 0.95, 0.96, 0.97, 0.98, 0.99, 1, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.29, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5 μg/L.

In one embodiment the sample is isolated postsurgically at two days, three days or more after surgery and determining a level of at least one biomarker comprises determining the level of PCT or fragment(s) thereof and a level of PCT or fragment(s) thereof above a threshold value±20% or more selected from a range of values in between 1 μg/L and 2 μg/L indicates an elevated likelihood of an adverse cardiovascular or cerebrovascular event, preferably a MACCE. Any value within this range may be considered as an appropriate threshold value. For example, 1, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.29, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5, 1.51, 1.52, 1.53, 1.54, 1.55, 1.56, 1.57, 1.58, 1.59, 1.6, 1.61, 1.62, 1.63, 1.64, 1.65, 1.66, 1.67, 1.68, 1.69, 1.7, 1.71, 1.72, 1.73, 1.74, 1.75, 1.76, 1.77, 1.78, 1.79, 1.8, 1.81, 1.82, 1.83, 1.84, 1.85, 1.86, 1.87, 1.88, 1.89, 1.9, 1.91, 1.92, 1.93, 1.94, 1.95, 1.96, 1.97, 1.98, 1.99, 2 μg/L.

In one embodiment a first sample is isolated from the patient postsurgically at a first time point, preferably one day or more after surgery, and a second sample is isolated from the patient postsurgically at a subsequent second time point, wherein preferably between the first and second time point at least 12 hours, 24 hours, 36 hours, 48 hours or more have passed and determining a level of at least one biomarker comprises determining the level of PCT or fragment(s) thereof and an increase or a leveling in the level of PCT or fragment(s) thereof indicates an elevated likelihood of an adverse cardiovascular or cerebrovascular event, preferably a MACCE.

In one embodiment a first sample is isolated from the patient postsurgically at a first time point, preferably one day or more after surgery, and a second sample is isolated from the patient postsurgically at a subsequent second time point, wherein preferably between the first and the second time point at least 12 hours, 24 hours, 36 hours, 48 hours or more have passed and determining a level of at least one biomarker comprises determining the level of PCT or fragment(s) thereof and an increase of the level of PCT or fragment(s) thereof above a threshold value±20% or more selected from a range of values in between 0.1 μg/L and 0.5 μg/L indicates an elevated likelihood of an adverse cardiovascular or cerebrovascular event, preferably a MACCE. Any value within this range may be considered as an appropriate threshold value for the increase of PCT or fragment(s) in between the first and second time point. For example, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5 μg/L.

### PCT/MINS:

In one embodiment determining a level of at least one biomarker comprises determining the level of PCT or fragment(s) thereof and a level of PCT or fragment(s) thereof above a threshold value indicates an elevated likelihood of an adverse cardiovascular or cerebrovascular event, preferably a MINS.

In one embodiment the sample is isolated postsurgically within 12 hours, preferably within 6 hours after surgery and determining a level of at least one biomarker comprises determining the level of PCT or fragment(s) thereof and a level of PCT or fragment(s) thereof above a threshold value±20% or more selected from a range of values in between 0.25 μg/L and 0.4 μg/L indicates an elevated likelihood of an adverse cardiovascular or cerebrovascular event, preferably a MINS. Any value within this range may be considered as an appropriate threshold value. For example, 0.25, 0.26, 0.27, 0.28, 0.29, 0.3, 0.31, 0.32, 0.33, 0.34, 0.35, 0.36, 0.37, 0.38, 0.39, 0.4 μg/L.

In one embodiment the sample is isolated postsurgically one day after surgery and determining a level of at least one biomarker comprises determining the level of PCT or fragment(s) thereof and a level of PCT or fragment(s) thereof above a threshold value±20% or more selected from a range of values in between 0.7 μg/L and 1.1 μg/L indicates an elevated likelihood of an adverse cardiovascular or cerebrovascular event, preferably a MINS. Any value within this range may be considered as an appropriate threshold value. For example, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1, 1.01, 1.02, 1.03, 1.04, 1.05, 1.06, 1.07, 1.08, 1.09, 1.1 μg/L.

In one embodiment the sample is isolated postsurgically two days after surgery and determining a level of at least one biomarker comprises determining the level of PCT or fragment(s) thereof and a level of PCT or fragment(s) thereof above a threshold value±20% or more selected from a range of values in between 0.8 μg/L and 1.3 μg/L indicates an elevated likelihood of an adverse cardiovascular or cerebrovascular event, preferably a MINS. Any value within this range may be considered as an appropriate threshold value. For example, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.29, 1.3 μg/L.

In one embodiment the sample is isolated postsurgically three days after surgery and determining a level of at least one biomarker comprises determining the level of PCT or fragment(s) thereof and a level of PCT or fragment(s) thereof above a threshold value±20% or more selected from a range of values in between 0.8 μg/L and 1.4 μg/L indicates an elevated likelihood of an adverse cardiovascular or cerebrovascular event, preferably a MINS. Any value within this range may be considered as an appropriate threshold value. For example, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.29, 1.3, 1.31, 1.32, 1.33, 1.34, 1.35, 1.36, 1.37, 1.38, 1.39, 1.4 μg/L.

In one embodiment the sample is isolated postsurgically at one day or more after surgery and determining a level of at least one biomarker comprises determining the level of PCT or fragment(s) thereof and a level of PCT or fragment(s) thereof above a threshold value±20% or more selected from a range of values in between 1 μg/L and 1.3 μg/L indicates an elevated likelihood of an adverse cardiovascular or cerebrovascular event, preferably a MINS. Any value within this range may be considered as an appropriate threshold value. For example, 1, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.29, 1.3 μg/L.

In one embodiment the sample is isolated postsurgically at two days, three days or more after surgery and determining a level of at least one biomarker comprises determining the level of PCT or fragment(s) thereof and a level of PCT or fragment(s) thereof above a threshold value±20% or more selected from a range of values in between 1 μg/L and 1.3 μg/L indicates an elevated likelihood of an adverse cardiovascular or cerebrovascular event, preferably a MINS. Any value within this range may be considered as an appropriate threshold value. For example, 1, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.29, 1.3 μg/L.

In one embodiment a first sample is isolated from the patient postsurgically at a first time point, preferably one day or more after surgery, and a second sample is isolated from the patient postsurgically at a subsequent second time point, wherein preferably between the first and second time point at least 12 hours, 24 hours, 36 hours, 48 hours or more have passed and determining a level of at least one biomarker comprises determining the level of PCT or fragment(s) thereof and an increase or a leveling in the level of PCT or fragment(s) thereof indicates an elevated likelihood of an adverse cardiovascular or cerebrovascular event, preferably a MINS.

In one embodiment a first sample is isolated from the patient postsurgically at a first time point, preferably one day or more after surgery, and a second sample is isolated from the patient postsurgically at a subsequent second time point, wherein preferably between the first and the second time point at least 12 hours, 24 hours, 36 hours, 48 hours or more have passed and determining a level of at least one biomarker comprises determining the level of PCT or fragment(s) thereof and an increase of the level of PCT or fragment(s) thereof above a threshold value±20% or more selected from a range of values in between 0.1 μg/L and 0.6 μg/L indicates an elevated likelihood of an adverse cardiovascular or cerebrovascular event, preferably a MINS. Any value within this range may be considered as an appropriate threshold value for the increase of PCT or fragment(s) in between the first and second time point. For example, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6 μg/L.

### Embodiments Relating to Determining a Level of proET-1 or Fragment(s) Thereof for Diagnosing or Predicting a Cardiovascular or Cerebrovascular Event

proET-1/MACCE:

In one embodiment determining a level of at least one biomarker comprises determining the level of proET-1 or fragment(s) thereof and a level of proET-1 or fragment(s) thereof above a threshold value indicates an elevated likelihood of an adverse cardiovascular or cerebrovascular event, preferably a MACCE.

In one embodiment the sample is isolated preoperatively and determining a level of at least one biomarker comprises determining the level of proET-1 or fragment(s) thereof and a level of proET-1 or fragment(s) thereof above a threshold value±20% or more selected from a range of values in between 60 pmol/L and 85 pmol/L indicates an elevated likelihood of an adverse cardiovascular or cerebrovascular event, preferably a MACCE. Any value within this range may be considered as an appropriate threshold value. For example, 60, 61, 62, 63, 64, 65, 66, 67, 68, 69, 70, 71, 72, 73, 74, 75, 76, 77, 78, 79, 80, 81, 82, 83, 84, 85 pmol/L.

In one embodiment the sample is isolated postsurgically within 12 hours, preferably within 6 hours after surgery and determining a level of at least one biomarker comprises determining the level of proET-1 or fragment(s) thereof and a level of proET-1 or fragment(s) thereof above a threshold value±20% or more selected from a range of values in between 80 pmol/L and 100 pmol/L indicates an elevated likelihood of an adverse cardiovascular or cerebrovascular event, preferably a MACCE. Any value within this range may be considered as an appropriate threshold value. For example, 80, 81, 82, 83, 84, 85, 86, 87, 88, 89, 90, 91, 92, 93, 94, 95, 96, 97, 98, 99, 100 pmol/L.

In one embodiment the sample is isolated postsurgically one day after surgery and determining a level of at least one biomarker comprises determining the level of proET-1 or fragment(s) thereof and a level of proET-1 or fragment(s) thereof above a threshold value±20% or more selected from a range of values in between 85 pmol/L and 105 pmol/L indicates an elevated likelihood of an adverse cardiovascular or cerebrovascular event, preferably a MACCE. Any value within this range may be considered as an appropriate threshold value. For example, 85, 86, 87, 88, 89, 90, 91, 92, 93, 94, 95, 96, 97, 98, 99, 100, 101, 102, 103, 104, 105 pmol/L.

In one embodiment the sample is isolated postsurgically two days after surgery and determining a level of at least one biomarker comprises determining the level of proET-1 or fragment(s) thereof and a level of proET-1 or fragment(s) thereof above a threshold value±20% or more selected from a range of values in between 85 pmol/L and 105 pmol/L indicates an elevated likelihood of an adverse cardiovascular or cerebrovascular event, preferably a MACCE. Any value within this range may be considered as an appropriate threshold value. For example, 85, 86, 87, 88, 89, 90, 91, 92, 93, 94, 95, 96, 97, 98, 99, 100, 101, 102, 103, 104, 105 pmol/L.

In one embodiment the sample is isolated postsurgically three days after surgery and determining a level of at least one biomarker comprises determining the level of proET-1 or fragment(s) thereof and a level of proET-1 or fragment(s) thereof above a threshold value±20% or more selected from a range of values in between 80 pmol/L and 105 pmol/L indicates an elevated likelihood of an adverse cardiovascular or cerebrovascular event, preferably a MACCE. Any value within this range may be considered as an appropriate threshold value. For example, 80, 81, 82, 83, 84, 85, 86, 87, 88, 89, 90, 91, 92, 93, 94, 95, 96, 97, 98, 99, 100, 101, 102, 103, 104, 105 pmol/L.

In one embodiment the sample is isolated postsurgically at one day, two days, three days or more after surgery and determining a level of at least one biomarker comprises determining the level of proET-1 or fragment(s) thereof and a level of proET-1 or fragment(s) thereof above a threshold value±20% or more selected from a range of values in between 85 pmol/L and 105 pmol/L indicates an elevated likelihood of an adverse cardiovascular or cerebrovascular event, preferably a MACCE. Any value within this range may be considered as an appropriate threshold value. For example, 85, 86, 87, 88, 89, 90, 91, 92, 93, 94, 95, 96, 97, 98, 99, 100, 101, 102, 103, 104, 105 pmol/L.

In one embodiment a first sample is isolated from the patient postsurgically at a first time point, preferably one day or more after surgery, and a second sample is isolated from the patient postsurgically at a subsequent second time point, wherein preferably between the first and the second time point at least 12 hours, 24 hours, 36 hours, 48 hours or more have passed and determining a level of at least one biomarker comprises determining the level of proET-1 or fragment(s) thereof and an increase or a leveling in the level of proET-1 or fragment(s) thereof indicates an elevated likelihood of an adverse cardiovascular or cerebrovascular event, preferably a MACCE.

In one embodiment a first sample is isolated from the patient postsurgically at a first time point, preferably one day or more after surgery, and a second sample is isolated from the patient postsurgically at a subsequent second time point, wherein preferably between the first and the second time point at least 12 hours, 24 hours, 36 hours, 48 hours or more have passed and determining a level of at least one biomarker comprises determining the level of proET-1 or fragment(s) thereof and an increase of the level of proET-1 or fragment(s) thereof above a threshold value±20% or more selected from a range of values in between 1 pmol/L and 10 pmol/L indicates an elevated likelihood of an adverse cardiovascular or cerebrovascular event, preferably a MACCE. Any value within this range may be considered as an appropriate threshold value for the increase of proET-1 or fragment(s) in between the first and second time point. For example, 1, 2, 3, 4, 5, 6, 7, 8, 9, 10 pmol/L.

proET-1/MINS:

In one embodiment determining a level of at least one biomarker comprises determining the level of proET-1 or fragment(s) thereof and a level of proET-1 or fragment(s) thereof above a threshold value indicates an elevated likelihood of an adverse cardiovascular or cerebrovascular event, preferably a MINS.

In one embodiment the sample is isolated preoperatively and determining a level of at least one biomarker comprises determining the level of proET-1 or fragment(s) thereof and a level of proET-1 or fragment(s) thereof above a threshold value±20% or more selected from a range of values in between 60 pmol/L and 85 pmol/L indicates an elevated likelihood of an adverse cardiovascular or cerebrovascular event, preferably a MINS. Any value within this range may be considered as an appropriate threshold value. For example, 60, 61, 62, 63, 64, 65, 66, 67, 68, 69, 70, 71, 72, 73, 74, 75, 76, 77, 78, 79, 80, 81, 82, 83, 84, 85 pmol/L.

In one embodiment the sample is isolated postsurgically within 12 hours, preferably within 6 hours after surgery and determining a level of at least one biomarker comprises determining the level of proET-1 or fragment(s) thereof and a level of proET-1 or fragment(s) thereof above a threshold value±20% or more selected from a range of values in between 75 pmol/L and 105 pmol/L indicates an elevated likelihood of an adverse cardiovascular or cerebrovascular event, preferably a MINS. Any value within this range may be considered as an appropriate threshold value. For example, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105 pmol/L.

In one embodiment the sample is isolated postsurgically one day after surgery and determining a level of at least one biomarker comprises determining the level of proET-1 or fragment(s) thereof and a level of proET-1 or fragment(s) thereof above a threshold value±20% or more selected from a range of values in between 75 pmol/L and 105 pmol/L indicates an elevated likelihood of an adverse cardiovascular or cerebrovascular event, preferably a MINS. Any value within this range may be considered as an appropriate threshold value. For example, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105 pmol/L.

In one embodiment the sample is isolated postsurgically two days after surgery and determining a level of at least one biomarker comprises determining the level of proET-1 or fragment(s) thereof and a level of proET-1 or fragment(s) thereof above a threshold value±20% or more selected from a range of values in between 75 pmol/L and 105 pmol/L indicates an elevated likelihood of an adverse cardiovascular or cerebrovascular event, preferably a MINS. Any value within this range may be considered as an appropriate threshold value. For example, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105 pmol/L.

In one embodiment the sample is isolated postsurgically three days after surgery and determining a level of at least one biomarker comprises determining the level of proET-1 or fragment(s) thereof and a level of proET-1 or fragment(s) thereof above a threshold value±20% or more selected from a range of values in between 75 pmol/L and 105 pmol/L indicates an elevated likelihood of an adverse cardiovascular or cerebrovascular event, preferably a MINS. Any value within this range may be considered as an appropriate threshold value. For example, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105 pmol/L.

In one embodiment the sample is isolated postsurgically at one day, two days, three days or more after surgery and determining a level of at least one biomarker comprises determining the level of proET-1 or fragment(s) thereof and a level of proET-1 or fragment(s) thereof above a threshold value±20% or more selected from a range of values in between 75 pmol/L and 105 pmol/L indicates an elevated likelihood of an adverse cardiovascular or cerebrovascular event, preferably a MINS. Any value within this range may be considered as an appropriate threshold value. For example, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105 pmol/L.

In one embodiment a first sample is isolated from the patient postsurgically at a first time point, preferably within 12 hours after surgery, and a second sample is isolated from the patient postsurgically at a subsequent second time point, preferably one day or two days after surgery, wherein preferably between the first and the second time point at least 12 hours, 24 hours or more have passed and determining a level of at least one biomarker comprises determining the level of proET-1 or fragment(s) thereof and an increase of the level of proET-1 or fragment(s) thereof above a threshold value±20% or more selected from a range of values in between 3 pmol/L and 20 pmol/L indicates an elevated likelihood of an adverse cardiovascular or cerebrovascular event, preferably a MINS. Any value within this range may be considered as an appropriate threshold value for the increase of proET-1 or fragment(s) in between the first and second time point. For example, 3, 4, 5, 6, 7, 8, 9, 10, 11, 12, 13, 14, 15, 16, 17, 18, 19, 20 pmol/L.

### Embodiments Relating to Determining a Level of proET-1, PCT or proADM or Fragment(s) Thereof for Diagnosing or Predicting a Cardiovascular or Cerebrovascular Event, which is a PMI

In one embodiment determining a level of at least one biomarker comprises determining the level of proET-1, PCT and/or proADM or fragment(s) thereof, and a level of proET-1, PCT and/or proADM or fragment(s) thereof above a threshold value indicates an elevated likelihood of an adverse cardiovascular or cerebrovascular event, preferably a perioperative myocardial injury (PMI). The sample may in some embodiments be obtained preoperatively or during surgery.

### Embodiments Relating to Determining a Level of proADM or Fragment(s) Thereof for Diagnosing or Predicting an Infection

### ADM/Any Infection:

In one embodiment determining a level of at least one biomarker comprises determining the level of proADM or fragment(s) thereof and a level of proADM or fragment(s) thereof above a threshold value indicates an elevated likelihood of an infection.

In one embodiment the sample is isolated preoperatively and determining a level of at least one biomarker comprises determining the level of proADM or fragment(s) thereof and a level of proADM or fragment(s) thereof above a threshold value±20% or more selected from a range of values in between 0.7 nmol/L and 0.8 nmol/L indicates an elevated likelihood of an infection. Any value within this range may be considered as an appropriate threshold value. For example, 0.70, 0.71, 0.72, 0.73, 0.74, 0.75, 0.76, 0.77, 0.78, 0.79, 0.8 nmol/L.

In one embodiment the sample is isolated postsurgically within 12 hours, preferably within 6 hours after surgery and determining a level of at least one biomarker comprises determining the level of proADM or fragment(s) thereof and a level of proADM or fragment(s) thereof above a threshold value±20% or more selected from a range of values in between 1.0 nmol/L and 1.2 nmol/L indicates an elevated likelihood of an infection. Any value within this range may be considered as an appropriate threshold value. For example, 1.0, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, 1.2 nmol/L.

In one embodiment the sample is isolated postsurgically one day after surgery and determining a level of at least one biomarker comprises determining the level of proADM or fragment(s) thereof and a level of proADM or fragment(s) thereof above a threshold value±20% or more selected from a range of values in between 1.3 nmol/L and 1.5 nmol/L indicates an elevated likelihood of an infection. Any value within this range may be considered as an appropriate threshold value. For example, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5 nmol/L.

In one embodiment the sample is isolated postsurgically two days after surgery and determining a level of at least one biomarker comprises determining the level of proADM or fragment(s) thereof and a level of proADM or fragment(s) thereof above a threshold value±20% or more selected from a range of values in between 1.3 nmol/L and 1.5 nmol/L indicates an elevated likelihood of an infection. Any value within this range may be considered as an appropriate threshold value. For example, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5 nmol/L.

In one embodiment the sample is isolated postsurgically three days after surgery and determining a level of at least one biomarker comprises determining the level of proADM or fragment(s) thereof and a level of proADM or fragment(s) thereof above a threshold value±20% or more selected from a range of values in between 1.1 nmol/L and 1.5 nmol/L indicates an elevated likelihood an infection. Any value within this range may be considered as an appropriate threshold value. For example, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.29, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5 nmol/L.

In one embodiment the sample isolated postsurgically and determining a level of at least one biomarker comprises determining the level of proADM or fragment(s) thereof and a level of proADM or fragment(s) thereof above a threshold value±20% or more selected from a range of values in between 1.1 nmol/L and 1.5 nmol/L indicates an elevated likelihood of an infection. Any value within this range may be considered as an appropriate threshold value. For example, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.29, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5 nmol/L.

In one embodiment the sample isolated postsurgically at one day, two days, three days or more after surgery and determining a level of at least one biomarker comprises determining the level of proADM or fragment(s) thereof and a level of proADM or fragment(s) thereof above a threshold value±20% or more selected from a range of values in between 1.3 nmol/L and 1.5 nmol/L indicates an elevated likelihood of an infection. Any value within this range may be considered as an appropriate threshold value. For example, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5 nmol/L.

In one embodiment a first sample is isolated from the patient postsurgically at a first time point, preferably one day or more after surgery, and a second sample is isolated from the patient postsurgically at a subsequent second time point, wherein preferably between the first and second time point at least 12 hours, 24 hours, 36 hours, 48 hours or more have passed and determining a level of at least one biomarker comprises determining the level of proADM or fragment(s) thereof and an increase or a leveling in the level of proADM or fragment(s) thereof indicates an elevated likelihood of an infection.

In one embodiment a first sample is isolated from the patient postsurgically at a first time point, preferably one day or more after surgery, and a second sample is isolated from the patient postsurgically at a subsequent second time point, wherein preferably between the first and the second time point at least 12 hours, 24 hours, 36 hours, 48 hours or more have passed and determining a level of at least one biomarker comprises determining the level of proADM or fragment(s) thereof and an increase of the level of proADM or fragment(s) thereof above a threshold value±20% or more selected from a range of values in between 0.01 nmol/L and 0.4 nmol/L indicates an elevated likelihood of an infection. Any value within this range may be considered as an appropriate threshold value for the increase of proADM or fragment(s) in between the first and second time point. For example, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4 nmol/L.

### ADM/Blood Infection:

In one embodiment determining a level of at least one biomarker comprises determining the level of proADM or fragment(s) thereof and a level of proADM or fragment(s) thereof above a threshold value indicates an elevated likelihood of a blood infection.

In one embodiment the sample is isolated preoperatively and determining a level of at least one biomarker comprises determining the level of proADM or fragment(s) thereof and a level of proADM or fragment(s) thereof above a threshold value±20% or more selected from a range of values in between 0.7 nmol/L and 0.9 nmol/L indicates an elevated likelihood of a blood infection. Any value within this range may be considered as an appropriate threshold value. For example, 0.70, 0.71, 0.72, 0.73, 0.74, 0.75, 0.76, 0.77, 0.78, 0.79, 0.8, 0.81, 0.82, 0.83, 0.84, 0.84, 0.85, 0.86, 0.87, 0.88. 0.89, 0.9 nmol/L.

In one embodiment the sample is isolated postsurgically within 12 hours, preferably within 6 hours after surgery and determining a level of at least one biomarker comprises determining the level of proADM or fragment(s) thereof and a level of proADM or fragment(s) thereof above a threshold value±20% or more selected from a range of values in between 1.0 nmol/L and 1.3 nmol/L indicates an elevated likelihood of a blood infection. Any value within this range may be considered as an appropriate threshold value. For example, 1.0, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.29, 1.3 nmol/L.

In one embodiment the sample is isolated postsurgically one day after surgery and determining a level of at least one biomarker comprises determining the level of proADM or fragment(s) thereof and a level of proADM or fragment(s) thereof above a threshold value±20% or more selected from a range of values in between 1.2 nmol/L and 1.8 nmol/L indicates an elevated likelihood of a blood infection. Any value within this range may be considered as an appropriate threshold value.

For example, 1.2, 1.21, 1.22, 1.23, 1.24, 1.25, 1.26, 1.27, 1.28, 1.29, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5, 1.51, 1.52, 1.53, 1.54, 1.55, 1.56, 1.57, 1.58, 1.59, 1.6, 1.61, 1.62, 1.63, 1.64, 1.65, 1.66, 1.67, 1.68, 1.69, 1.7, 1.71, 1.72, 1.73, 1.74, 1.75, 1.76, 1.77, 1.78, 1.79, 1.8 nmol/L.

In one embodiment the sample is isolated postsurgically two days after surgery and determining a level of at least one biomarker comprises determining the level of proADM or fragment(s) thereof and a level of proADM or fragment(s) thereof above a threshold value±20% or more selected from a range of values in between 1.2 nmol/L and 1.8 nmol/L indicates an elevated likelihood of a blood infection. Any value within this range may be considered as an appropriate threshold value. For example, 1.2, 1.21, 1.22, 1.23, 1.24, 1.25, 1.26, 1.27, 1.28, 1.29, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5, 1.51, 1.52, 1.53, 1.54, 1.55, 1.56, 1.57, 1.58, 1.59, 1.6, 1.61, 1.62, 1.63, 1.64, 1.65, 1.66, 1.67, 1.68, 1.69, 1.7, 1.71, 1.72, 1.73, 1.74, 1.75, 1.76, 1.77, 1.78, 1.79, 1.8 nmol/L.

In one embodiment the sample is isolated postsurgically three days after surgery and determining a level of at least one biomarker comprises determining the level of proADM or fragment(s) thereof and a level of proADM or fragment(s) thereof above a threshold value±20% or more selected from a range of values in between 1.2 nmol/L and 1.8 nmol/L indicates an elevated likelihood of a blood infection. Any value within this range may be considered as an appropriate threshold value. For example, 1.2, 1.21, 1.22, 1.23, 1.24, 1.25, 1.26, 1.27, 1.28, 1.29, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5, 1.51, 1.52, 1.53, 1.54, 1.55, 1.56, 1.57, 1.58, 1.59, 1.6, 1.61, 1.62, 1.63, 1.64, 1.65, 1.66, 1.67, 1.68, 1.69, 1.7, 1.71, 1.72, 1.73, 1.74, 1.75, 1.76, 1.77, 1.78, 1.79, 1.8 nmol/L.

In one embodiment the sample isolated postsurgically and determining a level of at least one biomarker comprises determining the level of proADM or fragment(s) thereof and a level of proADM or fragment(s) thereof above a threshold value±20% or more selected from a range of values in between 1.1 nmol/L and 1.5 nmol/L indicates an elevated likelihood of a blood infection. Any value within this range may be considered as an appropriate threshold value. For example, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.29, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5 nmol/L.

In one embodiment the sample isolated postsurgically at one day, two days, three days or more after surgery and determining a level of at least one biomarker comprises determining the level of proADM or fragment(s) thereof and a level of proADM or fragment(s) thereof above a threshold value±20% or more selected from a range of values in between 1.3 nmol/L and 1.8 nmol/L indicates an elevated likelihood of a blood infection. Any value within this range may be considered as an appropriate threshold value. For example, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5, 1.5, 1.51, 1.52, 1.53, 1.54, 1.55, 1.56, 1.57, 1.58, 1.59, 1.6, 1.61, 1.62, 1.63, 1.64, 1.65, 1.66, 1.67, 1.68, 1.69, 1.7, 1.71, 1.72, 1.73, 1.74, 1.75, 1.76, 1.77, 1.78, 1.79, 1.8 nmol/L.

In one embodiment a first sample is isolated from the patient postsurgically at a first time point, preferably one day or more after surgery, and a second sample is isolated from the patient postsurgically at a subsequent second time point, wherein preferably between the first and second time point at least 12 hours, 24 hours, 36 hours, 48 hours or more have passed and determining a level of at least one biomarker comprises determining the level of proADM or fragment(s) thereof and an increase or a leveling in the level of proADM or fragment(s) thereof indicates an elevated likelihood of a blood infection.

In one embodiment a first sample is isolated from the patient postsurgically at a first time point, preferably one day or more after surgery, and a second sample is isolated from the patient postsurgically at a subsequent second time point, wherein preferably between the first and the second time point at least 12 hours, 24 hours, 36 hours, 48 hours or more have passed and determining a level of at least one biomarker comprises determining the level of proADM or fragment(s) thereof and an increase of the level of proADM or fragment(s) thereof above a threshold value±20% or more selected from a range of values in between 0.01 nmol/L and 0.4 nmol/L indicates an elevated likelihood of a blood infection. Any value within this range may be considered as an appropriate threshold value for the increase of proADM or fragment(s) in between the first and second time point. For example, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4 nmol/L.

### PCT/Any Infection:

In one embodiment determining a level of at least one biomarker comprises determining the level of PCT or fragment(s) thereof and a level of PCT or fragment(s) thereof above a threshold value indicates an elevated likelihood of an infection.

In one embodiment the sample is isolated postsurgically within 12 hours, preferably within 6 hours after surgery and determining a level of at least one biomarker comprises determining the level of PCT or fragment(s) thereof and a level of PCT or fragment(s) thereof above a threshold value±20% or more selected from a range of values in between 0.2 μg/L and 0.3 μg/L indicates an elevated likelihood of an infection. Any value within this range may be considered as an appropriate threshold value. For example, 0.1, 0.2, 0.21, 0.22, 0.23, 0.24, 0.25, 0.26, 0.27, 0.28, 0.29, 0.3 μg/L.

In one embodiment the sample is isolated postsurgically one day after surgery and determining a level of at least one biomarker comprises determining the level of PCT or fragment(s) thereof and a level of PCT or fragment(s) thereof above a threshold value±20% or more selected from a range of values in between 0.6 μg/L and 0.9 μg/L indicates an elevated likelihood of an infection. Any value within this range may be considered as an appropriate threshold value. For example, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9 μg/L.

In one embodiment the sample is isolated postsurgically two days after surgery and determining a level of at least one biomarker comprises determining the level of PCT or fragment(s) thereof and a level of PCT or fragment(s) thereof above a threshold value±20% or more selected from a range of values in between 0.7 μg/L and 1.3 μg/L indicates an elevated likelihood of an infection. Any value within this range may be considered as an appropriate threshold value. For example, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.29, 1.3, μg/L.

In one embodiment the sample is isolated postsurgically three days after surgery and determining a level of at least one biomarker comprises determining the level of PCT or fragment(s) thereof and a level of PCT or fragment(s) thereof above a threshold value±20% or more selected from a range of values in between 0.6 μg/L and 1.3 μg/L indicates an elevated likelihood of an infection. Any value within this range may be considered as an appropriate threshold value. For example, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.29, 1.3 μg/L.

In one embodiment the sample is isolated postsurgically at one day or more after surgery and determining a level of at least one biomarker comprises determining the level of PCT or fragment(s) thereof and a level of PCT or fragment(s) thereof above a threshold value±20% or more selected from a range of values in between 0.6 μg/L and 1.1 μg/L indicates an elevated likelihood of an infection. Any value within this range may be considered as an appropriate threshold value. For example, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1, 1.01, 1.02, 1.03, 1.04, 1.05, 1.06, 1.07, 1.08, 1.09, 1.1 μg/L.

In one embodiment the sample is isolated postsurgically at two days, three days or more after surgery and determining a level of at least one biomarker comprises determining the level of PCT or fragment(s) thereof and a level of PCT or fragment(s) thereof above a threshold value±20% or more selected from a range of values in between 0.7 μg/L and 1.3 μg/L indicates an elevated likelihood of an infection. Any value within this range may be considered as an appropriate threshold value. For example, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.29, 1.3 μg/L.

In one embodiment a first sample is isolated from the patient postsurgically at a first time point, preferably one day or more after surgery, and a second sample is isolated from the patient postsurgically at a subsequent second time point, wherein preferably between the first and second time point at least 12 hours, 24 hours, 36 hours, 48 hours or more have passed and determining a level of at least one biomarker comprises determining the level of PCT or fragment(s) thereof and an increase in the level of PCT or fragment(s) thereof indicates an elevated likelihood of an infection.

In one embodiment a first sample is isolated from the patient postsurgically at a first time point, preferably one day or more after surgery, and a second sample is isolated from the patient postsurgically at a subsequent second time point, wherein preferably between the first and the second time point at least 12 hours, 24 hours, 36 hours, 48 hours or more have passed and determining a level of at least one biomarker comprises determining the level of PCT or fragment(s) thereof and an increase of the level of PCT or fragment(s) thereof above a threshold value±20% or more selected from a range of values in between 0.1 μg/L and 0.6 μg/L indicates an elevated likelihood of an infection. Any value within this range may be considered as an appropriate threshold value for the increase of PCT or fragment(s) in between the first and second time point. For example, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6 μg/L.

### PCT/Blood Infection:

In one embodiment determining a level of at least one biomarker comprises determining the level of PCT or fragment(s) thereof and a level of PCT or fragment(s) thereof above a threshold value indicates an elevated likelihood of a blood infection.

In one embodiment the sample is isolated postsurgically within 12 hours, preferably within 6 hours after surgery and determining a level of at least one biomarker comprises determining the level of PCT or fragment(s) thereof and a level of PCT or fragment(s) thereof above a threshold value±20% or more selected from a range of values in between 0.1 μg/L and 0.3 μg/L indicates an elevated likelihood of a blood infection. Any value within this range may be considered as an appropriate threshold value. For example, 0.1, 0.11, 0.12, 0.13, 0.14, 0.15, 0.16, 0.17, 0.18, 0.19, 0.2, 0.2, 0.21, 0.22, 0.23, 0.24, 0.25, 0.26, 0.27, 0.28, 0.29, 0.3 μg/L.

In one embodiment the sample is isolated postsurgically one day after surgery and determining a level of at least one biomarker comprises determining the level of PCT or fragment(s) thereof and a level of PCT or fragment(s) thereof above a threshold value±20% or more selected from a range of values in between 0.6 μg/L and 1.8 μg/L indicates an elevated likelihood of an blood infection. Any value within this range may be considered as an appropriate threshold value. For example, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.29, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5, 1.5, 1.51, 1.52, 1.53, 1.54, 1.55, 1.56, 1.57, 1.58, 1.59, 1.6, 1.61, 1.62, 1.63, 1.64, 1.65, 1.66, 1.67, 1.68, 1.69, 1.7, 1.71, 1.72, 1.73, 1.74, 1.75, 1.76, 1.77, 1.78, 1.79, 1.8 μg/L.

In one embodiment the sample is isolated postsurgically two days after surgery and determining a level of at least one biomarker comprises determining the level of PCT or fragment(s) thereof and a level of PCT or fragment(s) thereof above a threshold value±20% or more selected from a range of values in between 0.7 μg/L and 2.3 μg/L indicates an elevated likelihood of a blood infection. Any value within this range may be considered as an appropriate threshold value. For example, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.29, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5, 1.5, 1.51, 1.52, 1.53, 1.54, 1.55, 1.56, 1.57, 1.58, 1.59, 1.6, 1.61, 1.62, 1.63, 1.64, 1.65, 1.66, 1.67, 1.68, 1.69, 1.7, 1.71, 1.72, 1.73, 1.74, 1.75, 1.76, 1.77, 1.78, 1.79, 1.8, 1.81, 1.82, 1.83, 1.84, 1.85, 1.86, 1.87, 1.88, 1.89, 1.9, 1.91, 1.92, 1.93, 1.94, 1.95, 1.96, 1.97, 1.98, 1.99, 2, 2.01, 2.02, 2.03, 2.04, 2.05, 2.06, 2.07, 2.08, 2.09, 2.1, 2.11, 2.12, 2.13, 2.14, 2.15, 2.16, 2.17, 2.18, 2.19, 2.2, 2.21, 2.22, 2.23, 2.24, 2.25, 2.26, 2.27, 2.28, 2.29, 2.3 μg/L.

In one embodiment the sample is isolated postsurgically three days after surgery and determining a level of at least one biomarker comprises determining the level of PCT or fragment(s) thereof and a level of PCT or fragment(s) thereof above a threshold value±20% or more selected from a range of values in between 0.7 μg/L and 2.3 μg/L indicates an elevated likelihood of a blood infection. Any value within this range may be considered as an appropriate threshold value. For example, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.29, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5, 1.5, 1.51, 1.52, 1.53, 1.54, 1.55, 1.56, 1.57, 1.58, 1.59, 1.6, 1.61, 1.62, 1.63, 1.64, 1.65, 1.66, 1.67, 1.68, 1.69, 1.7, 1.71, 1.72, 1.73, 1.74, 1.75, 1.76, 1.77, 1.78, 1.79, 1.8, 1.8, 1.81, 1.82, 1.83, 1.84, 1.85, 1.86, 1.87, 1.88, 1.89, 1.9, 1.91, 1.92, 1.93, 1.94, 1.95, 1.96, 1.97, 1.98, 1.99, 2, 2.01, 2.02, 2.03, 2.04, 2.05, 2.06, 2.07, 2.08, 2.09, 2.1, 2.11, 2.12, 2.13, 2.14, 2.15, 2.16, 2.17, 2.18, 2.19, 2.2, 2.21, 2.22, 2.23, 2.24, 2.25, 2.26, 2.27, 2.28, 2.29, 2.3 μg/L.

In one embodiment the sample is isolated postsurgically at one day or more after surgery and determining a level of at least one biomarker comprises determining the level of PCT or fragment(s) thereof and a level of PCT or fragment(s) thereof above a threshold value±20% or more selected from a range of values in between 0.7 μg/L and 1.8 μg/L indicates an elevated likelihood of an blood infection. Any value within this range may be considered as an appropriate threshold value. For example, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.29, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5, 1.5, 1.51, 1.52, 1.53, 1.54, 1.55, 1.56, 1.57, 1.58, 1.59, 1.6, 1.61, 1.62, 1.63, 1.64, 1.65, 1.66, 1.67, 1.68, 1.69, 1.7, 1.71, 1.72, 1.73, 1.74, 1.75, 1.76, 1.77, 1.78, 1.79, 1.8 μg/L.

In one embodiment the sample is isolated postsurgically at two days, three days or more after surgery and determining a level of at least one biomarker comprises determining the level of PCT or fragment(s) thereof and a level of PCT or fragment(s) thereof above a threshold value±20% or more selected from a range of values in between 1 μg/L and 2.3 μg/L indicates an elevated likelihood of a blood infection. Any value within this range may be considered as an appropriate threshold value. For example, 1, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.29, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5, 1.5, 1.51, 1.52, 1.53, 1.54, 1.55, 1.56, 1.57, 1.58, 1.59, 1.6, 1.61, 1.62, 1.63, 1.64, 1.65, 1.66, 1.67, 1.68, 1.69, 1.7, 1.71, 1.72, 1.73, 1.74, 1.75, 1.76, 1.77, 1.78, 1.79, 1.8, 1.81, 1.82, 1.83, 1.84, 1.85, 1.86, 1.87, 1.88, 1.89, 1.9, 1.91, 1.92, 1.93, 1.94, 1.95, 1.96, 1.97, 1.98, 1.99, 2, 2.01, 2.02, 2.03, 2.04, 2.05, 2.06, 2.07, 2.08, 2.09, 2.1, 2.11, 2.12, 2.13, 2.14, 2.15, 2.16, 2.17, 2.18, 2.19, 2.2, 2.21, 2.22, 2.23, 2.24, 2.25, 2.26, 2.27, 2.28, 2.29, 2.3 μg/L.

In one embodiment a first sample is isolated from the patient postsurgically at a first time point, preferably one day or more after surgery, and a second sample is isolated from the patient postsurgically at a subsequent second time point, wherein preferably between the first and second time point at least 12 hours, 24 hours, 36 hours, 48 hours or more have passed and determining a level of at least one biomarker comprises determining the level of PCT or fragment(s) thereof and an increase in the level of PCT or fragment(s) thereof indicates an elevated likelihood of a blood infection.

In one embodiment a first sample is isolated from the patient postsurgically at a first time point, preferably one day or more after surgery, and a second sample is isolated from the patient postsurgically at a subsequent second time point, wherein preferably between the first and the second time point at least 12 hours, 24 hours, 36 hours, 48 hours or more have passed and determining a level of at least one biomarker comprises determining the level of PCT or fragment(s) thereof and an increase of the level of PCT or fragment(s) thereof above a threshold value±20% or more selected from a range of values in between 0.1 μg/L and 1 μg/L indicates an elevated likelihood of a blood infection. Any value within this range may be considered as an appropriate threshold value for the increase of PCT or fragment(s) in between the first and second time point. For example, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1 μg/L.

proET-1/Any Infection:

In one embodiment determining a level of at least one biomarker comprises determining the level of proET-1 or fragment(s) thereof and a level of proET-1 or fragment(s) thereof above a threshold value indicates an elevated likelihood of an infection.

In one embodiment the sample is isolated postsurgically two days after surgery and determining a level of at least one biomarker comprises determining the level of proET-1 or fragment(s) thereof and a level of proET-1 or fragment(s) thereof above a threshold value±20% or more selected from a range of values in between 85 pmol/L and 95 pmol/L indicates an elevated likelihood of an infection. Any value within this range may be considered as an appropriate threshold value. For example, 85, 86, 87, 88, 89, 90, 91, 92, 93, 94, 95 pmol/L.

In one embodiment the sample is isolated postsurgically three days after surgery and determining a level of at least one biomarker comprises determining the level of proET-1 or fragment(s) thereof and a level of proET-1 or fragment(s) thereof above a threshold value±20% or more selected from a range of values in between 80 pmol/L and 95 pmol/L indicates an elevated likelihood of an infection. Any value within this range may be considered as an appropriate threshold value. For example, 80, 81, 82, 83, 84, 85, 86, 87, 88, 89, 90, 91, 92, 93, 94, 95 pmol/L.

In one embodiment the sample is isolated postsurgically two days, three days or more after surgery and determining a level of at least one biomarker comprises determining the level of proET-1 or fragment(s) thereof and a level of proET-1 or fragment(s) thereof above a threshold value±20% or more selected from a range of values in between 80 pmol/L and 95 pmol/L indicates an elevated likelihood of an infection. Any value within this range may be considered as an appropriate threshold value. For example, 80, 81, 82, 83, 84, 85, 86, 87, 88, 89, 90, 91, 92, 93, 94, 95 pmol/L.

proET-1/Blood Infection:

In one embodiment determining a level of at least one biomarker comprises determining the level of proET-1 or fragment(s) thereof and a level of proET-1 or fragment(s) thereof above a threshold value indicates an elevated likelihood of a blood infection.

In one embodiment the sample is isolated postsurgically two days after surgery and determining a level of at least one biomarker comprises determining the level of proET-1 or fragment(s) thereof and a level of proET-1 or fragment(s) thereof above a threshold value±20% or more selected from a range of values in between 85 pmol/L and 105 pmol/L indicates an elevated likelihood of a blood infection. Any value within this range may be considered as an appropriate threshold value. For example, 85, 86, 87, 88, 89, 90, 91, 92, 93, 94, 95, 96, 97, 98, 99, 100, 101, 102, 103, 104, 105 pmol/L.

In one embodiment the sample is isolated postsurgically three days after surgery and determining a level of at least one biomarker comprises determining the level of proET-1 or fragment(s) thereof and a level of proET-1 or fragment(s) thereof above a threshold value±20% or more selected from a range of values in between 80 pmol/L and 110 pmol/L indicates an elevated likelihood of a blood infection. Any value within this range may be considered as an appropriate threshold value. For example, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110 pmol/L.

In one embodiment the sample is isolated postsurgically two days, three days or more after surgery and determining a level of at least one biomarker comprises determining the level of proET-1 or fragment(s) thereof and a level of proET-1 or fragment(s) thereof above a threshold value±20% or more selected from a range of values in between 80 pmol/L and 110 pmol/L indicates an elevated likelihood of a blood infection. Any value within this range may be considered as an appropriate threshold value. For example, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110 pmol/L.

### ADM/Infection and MACCE/MINS:

In one embodiment determining a level of at least one biomarker comprises determining the level of proADM or fragment(s) thereof and a level of proADM or fragment(s) thereof above a threshold value indicates an elevated likelihood of an infection, preferably a blood infection, and an adverse cardiovascular or cerebrovascular event, preferably a MACCE and/or a MINS.

In one embodiment the sample is isolated preoperatively and determining a level of at least one biomarker comprises determining the level of proADM or fragment(s) thereof and a level of proADM or fragment(s) thereof above a threshold value±20% or more selected from a range of values in between 0.7 nmol/L and 0.9 nmol/L indicates an elevated likelihood of an infection, preferably a blood infection, and an adverse cardiovascular or cerebrovascular event, preferably a MACCE and/or a MINS. Any value within this range may be considered as an appropriate threshold value. For example, 0.70, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9 nmol/L.

In one embodiment the sample is isolated postsurgically within 12 hours, preferably within 6 hours after surgery and determining a level of at least one biomarker comprises determining the level of proADM or fragment(s) thereof and a level of proADM or fragment(s) thereof above a threshold value±20% or more selected from a range of values in between 1.0 nmol/L and 1.6 nmol/L indicates an elevated likelihood of an infection, preferably a blood infection, and an adverse cardiovascular or cerebrovascular event, preferably a MACCE and/or a MINS. Any value within this range may be considered as an appropriate threshold value. For example, 1, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.29, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5, 1.51, 1.52, 1.53, 1.54, 1.55, 1.56, 1.57, 1.58, 1.59, 1.6 nmol/L.

In one embodiment the sample is isolated postsurgically one day after surgery and determining a level of at least one biomarker comprises determining the level of proADM or fragment(s) thereof and a level of proADM or fragment(s) thereof above a threshold value±20% or more selected from a range of values in between 1.3 nmol/L and 2.2. nmol/L indicates an elevated likelihood of an infection, preferably a blood infection, and an adverse cardiovascular or cerebrovascular event, preferably a MACCE and/or a MINS. Any value within this range may be considered as an appropriate threshold value. For example, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5, 1.51, 1.52, 1.53, 1.54, 1.55, 1.56, 1.57, 1.58, 1.59, 1.6, 1.61, 1.62, 1.63, 1.64, 1.65, 1.66, 1.67, 1.68, 1.69, 1.7, 1.71, 1.72, 1.73, 1.74, 1.75, 1.76, 1.77, 1.78, 1.79, 1.8, 1.81, 1.82, 1.83, 1.84, 1.85, 1.86, 1.87, 1.88, 1.89, 1.9, 1.91, 1.92, 1.93, 1.94, 1.95, 1.96, 1.97, 1.98, 1.99, 2, 2.01, 2.02, 2.03, 2.04, 2.05, 2.06, 2.07, 2.08, 2.09, 2.1, 2.11, 2.12, 2.13, 2.14, 2.15, 2.16, 2.17, 2.18, 2.19, 2.2 nmol/L.

In one embodiment the sample is isolated postsurgically two days after surgery and determining a level of at least one biomarker comprises determining the level of proADM or fragment(s) thereof and a level of proADM or fragment(s) thereof above a threshold value±20% or more selected from a range of values in between 1.3 nmol/L and 2.3 nmol/L indicates an elevated likelihood of an infection, preferably a blood infection, and an adverse cardiovascular or cerebrovascular event, preferably a MACCE and/or a MINS. Any value within this range may be considered as an appropriate threshold value. For example, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5, 1.51, 1.52, 1.53, 1.54, 1.55, 1.56, 1.57, 1.58, 1.59, 1.6, 1.61, 1.62, 1.63, 1.64, 1.65, 1.66, 1.67, 1.68, 1.69, 1.7, 1.71, 1.72, 1.73, 1.74, 1.75, 1.76, 1.77, 1.78, 1.79, 1.8, 1.81, 1.82, 1.83, 1.84, 1.85, 1.86, 1.87, 1.88, 1.89, 1.9, 1.91, 1.92, 1.93, 1.94, 1.95, 1.96, 1.97, 1.98, 1.99, 2, 2.01, 2.02, 2.03, 2.04, 2.05, 2.06, 2.07, 2.08, 2.09, 2.1, 2.11, 2.12, 2.13, 2.14, 2.15, 2.16, 2.17, 2.18, 2.19, 2.2, 2.21, 2.22, 2.23, 2.24, 2.25, 2.26, 2.27, 2.28, 2.29, 2.3 nmol/L.

In one embodiment the sample is isolated postsurgically three days after surgery and determining a level of at least one biomarker comprises determining the level of proADM or fragment(s) thereof and a level of proADM or fragment(s) thereof above a threshold value±20% or more selected from a range of values in between 1.3 nmol/L and 2.3 nmol/L indicates an elevated likelihood of an infection, preferably a blood infection, and an adverse cardiovascular or cerebrovascular event, preferably a MACCE and/or a MINS. Any value within this range may be considered as an appropriate threshold value. For example, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5, 1.51, 1.52, 1.53, 1.54, 1.55, 1.56, 1.57, 1.58, 1.59, 1.6, 1.61, 1.62, 1.63, 1.64, 1.65, 1.66, 1.67, 1.68, 1.69, 1.7, 1.71, 1.72, 1.73, 1.74, 1.75, 1.76, 1.77, 1.78, 1.79, 1.8, 1.81, 1.82, 1.83, 1.84, 1.85, 1.86, 1.87, 1.88, 1.89, 1.9, 1.91, 1.92, 1.93, 1.94, 1.95, 1.96, 1.97, 1.98, 1.99, 2, 2.01, 2.02, 2.03, 2.04, 2.05, 2.06, 2.07, 2.08, 2.09, 2.1, 2.11, 2.12, 2.13, 2.14, 2.15, 2.16, 2.17, 2.18, 2.19, 2.2, 2.21, 2.22, 2.23, 2.24, 2.25, 2.26, 2.27, 2.28, 2.29, 2.3 nmol/L.

In one embodiment the sample is isolated postsurgically and determining a level of at least one biomarker comprises determining the level of proADM or fragment(s) thereof and a level of proADM or fragment(s) thereof above a threshold value±20% or more selected from a range of values in between 1.3 nmol/L and 2 nmol/L indicates an elevated likelihood of an infection, preferably a blood infection, and an adverse cardiovascular or cerebrovascular event, preferably a MACCE and/or a MINS. Any value within this range may be considered as an appropriate threshold value. For example, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5, 1.51, 1.52, 1.53, 1.54, 1.55, 1.56, 1.57, 1.58, 1.59, 1.6, 1.61, 1.62, 1.63, 1.64, 1.65, 1.66, 1.67, 1.68, 1.69, 1.7, 1.71, 1.72, 1.73, 1.74, 1.75, 1.76, 1.77, 1.78, 1.79, 1.8, 1.81, 1.82, 1.83, 1.84, 1.85, 1.86, 1.87, 1.88, 1.89, 1.9, 1.91, 1.92, 1.93, 1.94, 1.95, 1.96, 1.97, 1.98, 1.99, 2 nmol/L.

In one embodiment the sample is isolated postsurgically three days after surgery and determining a level of at least one biomarker comprises determining the level of proADM or fragment(s) thereof and a level of proADM or fragment(s) thereof above a threshold value±20% or more selected from a range of values in between 1.3 nmol/L and 2.3 nmol/L indicates an elevated likelihood of an infection, preferably a blood infection, and an adverse cardiovascular or cerebrovascular event, preferably a MACCE and/or a MINS. Any value within this range may be considered as an appropriate threshold value. For example, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5, 1.51, 1.52, 1.53, 1.54, 1.55, 1.56, 1.57, 1.58, 1.59, 1.6, 1.61, 1.62, 1.63, 1.64, 1.65, 1.66, 1.67, 1.68, 1.69, 1.7, 1.71, 1.72, 1.73, 1.74, 1.75, 1.76, 1.77, 1.78, 1.79, 1.8, 1.81, 1.82, 1.83, 1.84, 1.85, 1.86, 1.87, 1.88, 1.89, 1.9, 1.91, 1.92, 1.93, 1.94, 1.95, 1.96, 1.97, 1.98, 1.99, 2, 2.01, 2.02, 2.03, 2.04, 2.05, 2.06, 2.07, 2.08, 2.09, 2.1, 2.11, 2.12, 2.13, 2.14, 2.15, 2.16, 2.17, 2.18, 2.19, 2.2, 2.21, 2.22, 2.23, 2.24, 2.25, 2.26, 2.27, 2.28, 2.29, 2.3 nmol/L.

In one embodiment a first sample is isolated from the patient postsurgically at a first time point, preferably one day or more after surgery, and a second sample is isolated from the patient postsurgically at a subsequent second time point, wherein preferably between the first and second time point at least 12 hours, 24 hours, 36 hours, 48 hours or more have passed and determining a level of at least one biomarker comprises determining the level of proADM or fragment(s) thereof and an increase in the level of proADM or fragment(s) thereof indicates an elevated likelihood an infection, preferably a blood infection, and an adverse cardiovascular or cerebrovascular event, preferably a MACCE and/or a MINS.

In one embodiment a first sample is isolated from the patient postsurgically at a first time point, preferably one day or more after surgery, and a second sample is isolated from the patient postsurgically at a subsequent second time point, wherein preferably between the first and the second time point at least 12 hours, 24 hours, 36 hours, 48 hours or more have passed and determining a level of at least one biomarker comprises determining the level of proADM or fragment(s) thereof and an increase of the level of proADM or fragment(s) thereof above a threshold value±20% or more selected from a range of values in between 0.01 nmol/L and 1 nmol/L indicates an elevated likelihood an infection, preferably a blood infection, and an adverse cardiovascular or cerebrovascular event, preferably a MACCE and/or a MINS. Any value within this range may be considered as an appropriate threshold value for the increase of proADM or fragment(s) in between the first and second time point. For example, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1 nmol/L.

### PCT/Infection and MACCE/MINS:

In one embodiment determining a level of at least one biomarker comprises determining the level of PCT or fragment(s) thereof and a level of PCT or fragment(s) thereof above a threshold value indicates an elevated likelihood of an infection, preferably a blood infection, and an adverse cardiovascular or cerebrovascular event, preferably a MACCE and/or a MINS.

In one embodiment the sample is isolated postsurgically within 12 hours, preferably within 6 hours after surgery and determining a level of at least one biomarker comprises determining the level of PCT or fragment(s) thereof and a level of PCT or fragment(s) thereof above a threshold value±20% or more selected from a range of values in between 0.1 μg/L and 0.4 μg/L indicates an elevated likelihood of an infection, preferably a blood infection, and an adverse cardiovascular or cerebrovascular event, preferably a MACCE and/or a MINS. Any value within this range may be considered as an appropriate threshold value. For example, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4 μg/L.

In one embodiment the sample is isolated postsurgically one day after surgery and determining a level of at least one biomarker comprises determining the level of PCT or fragment(s) thereof and a level of PCT or fragment(s) thereof above a threshold value±20% or more selected from a range of values in between 0.6 μg/L and 1.8 μg/L indicates an elevated likelihood of an infection, preferably a blood infection, and an adverse cardiovascular or cerebrovascular event, preferably a MACCE and/or a MINS. Any value within this range may be considered as an appropriate threshold value. For example, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.291.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5, 1.51, 1.52, 1.53, 1.54, 1.55, 1.56, 1.57, 1.58, 1.59, 1.6, 1.61, 1.62, 1.63, 1.64, 1.65, 1.66, 1.67, 1.68, 1.69, 1.7, 1.71, 1.72, 1.73, 1.74, 1.75, 1.76, 1.77, 1.78, 1.79, 1.8 μg/L.

In one embodiment the sample is isolated postsurgically two days after surgery and determining a level of at least one biomarker comprises determining the level of PCT or fragment(s) thereof and a level of PCT or fragment(s) thereof above a threshold value±20% or more selected from a range of values in between 0.6 μg/L and 3 μg/L indicates an elevated likelihood of an infection, preferably a blood infection, and an adverse cardiovascular or cerebrovascular event, preferably a MACCE and/or a MINS. Any value within this range may be considered as an appropriate threshold value. For example, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.291.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5, 1.51, 1.52, 1.53, 1.54, 1.55, 1.56, 1.57, 1.58, 1.59, 1.6, 1.61, 1.62, 1.63, 1.64, 1.65, 1.66, 1.67, 1.68, 1.69, 1.7, 1.71, 1.72, 1.73, 1.74, 1.75, 1.76, 1.77, 1.78, 1.79, 1.8, 1.81, 1.82, 1.83, 1.84, 1.85, 1.86, 1.87, 1.88, 1.89, 1.9, 1.91, 1.92, 1.93, 1.94, 1.95, 1.96, 1.97, 1.98, 1.99, 2, 2.01, 2.02, 2.03, 2.04, 2.05, 2.06, 2.07, 2.08, 2.09, 2.1, 2.11, 2.12, 2.13, 2.14, 2.15, 2.16, 2.17, 2.18, 2.19, 2.2, 2.21, 2.22, 2.23, 2.24, 2.25, 2.26, 2.27, 2.28, 2.29, 2.3, 2.31, 2.32, 2.33, 2.34, 2.35, 2.36, 2.37, 2.38, 2.39, 2.4, 2.41, 2.42, 2.43, 2.44, 2.45, 2.46, 2.47, 2.48, 2.49, 2.5, 2.51, 2.52, 2.53, 2.54, 2.55, 2.56, 2.57, 2.58, 2.59, 2.6, 2.61, 2.62, 2.63, 2.64, 2.65, 2.66, 2.67, 2.68, 2.69, 2.7, 2.71, 2.72, 2.73, 2.74, 2.75, 2.76, 2.77, 2.78, 2.79, 2.8, 2.81, 2.82, 2.83, 2.84, 2.85, 2.86, 2.87, 2.88, 2.89, 2.9, 2.91, 2.92, 2.93, 2.94, 2.95, 2.96, 2.97, 2.98, 2.99, 3 μg/L.

In one embodiment the sample is isolated postsurgically three days after surgery and determining a level of at least one biomarker comprises determining the level of PCT or fragment(s) thereof and a level of PCT or fragment(s) thereof above a threshold value±20% or more selected from a range of values in between 0.6 μg/L and 3 μg/L indicates an elevated likelihood of an infection, preferably a blood infection, and an adverse cardiovascular or cerebrovascular event, preferably a MACCE and/or a MINS. Any value within this range may be considered as an appropriate threshold value. For example, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.291.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5, 1.51, 1.52, 1.53, 1.54, 1.55, 1.56, 1.57, 1.58, 1.59, 1.6, 1.61, 1.62, 1.63, 1.64, 1.65, 1.66, 1.67, 1.68, 1.69, 1.7, 1.71, 1.72, 1.73, 1.74, 1.75, 1.76, 1.77, 1.78, 1.79, 1.8, 1.81, 1.82, 1.83, 1.84, 1.85, 1.86, 1.87, 1.88, 1.89, 1.9, 1.91, 1.92, 1.93, 1.94, 1.95, 1.96, 1.97, 1.98, 1.99 2, 2.01, 2.02, 2.03, 2.04, 2.05, 2.06, 2.07, 2.08, 2.09, 2.1, 2.11, 2.12, 2.13, 2.14, 2.15, 2.16, 2.17, 2.18, 2.19, 2.2, 2.21, 2.22, 2.23, 2.24, 2.25, 2.26, 2.27, 2.28, 2.29, 2.3, 2.31, 2.32, 2.33, 2.34, 2.35, 2.36, 2.37, 2.38, 2.39, 2.4, 2.41, 2.42, 2.43, 2.44, 2.45, 2.46, 2.47, 2.48, 2.49, 2.5, 2.51, 2.52, 2.53, 2.54, 2.55, 2.56, 2.57, 2.58, 2.59, 2.6, 2.61, 2.62, 2.63, 2.64, 2.65, 2.66, 2.67, 2.68, 2.69, 2.7, 2.71, 2.72, 2.73, 2.74, 2.75, 2.76, 2.77, 2.78, 2.79, 2.8, 2.81, 2.82, 2.83, 2.84, 2.85, 2.86, 2.87, 2.88, 2.89, 2.9, 2.91, 2.92, 2.93, 2.94, 2.95, 2.96, 2.97, 2.98, 2.99, 3 μg/L.

In one embodiment the sample is isolated postsurgically and determining a level of at least one biomarker comprises determining the level of PCT or fragment(s) thereof and a level of PCT or fragment(s) thereof above a threshold value±20% or more selected from a range of values in between 0.7 μg/L and 3 μg/L indicates an elevated likelihood of an infection, preferably a blood infection, and an adverse cardiovascular or cerebrovascular event, preferably a MACCE and/or a MINS. Any value within this range may be considered as an appropriate threshold value. For example, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.291.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5, 1.51, 1.52, 1.53, 1.54, 1.55, 1.56, 1.57, 1.58, 1.59, 1.6, 1.61, 1.62, 1.63, 1.64, 1.65, 1.66, 1.67, 1.68, 1.69, 1.7, 1.71, 1.72, 1.73, 1.74, 1.75, 1.76, 1.77, 1.78, 1.79, 1.8, 1.81, 1.82, 1.83, 1.84, 1.85, 1.86, 1.87, 1.88, 1.89, 1.9, 1.91, 1.92, 1.93, 1.94, 1.95, 1.96, 1.97, 1.98, 1.99, 2, 2.01, 2.02, 2.03, 2.04, 2.05, 2.06, 2.07, 2.08, 2.09, 2.1, 2.11, 2.12, 2.13, 2.14, 2.15, 2.16, 2.17, 2.18, 2.19, 2.2, 2.21, 2.22, 2.23, 2.24, 2.25, 2.26, 2.27, 2.28, 2.29, 2.3, 2.31, 2.32, 2.33, 2.34, 2.35, 2.36, 2.37, 2.38, 2.39, 2.4, 2.41, 2.42, 2.43, 2.44, 2.45, 2.46, 2.47, 2.48, 2.49, 2.5, 2.51, 2.52, 2.53, 2.54, 2.55, 2.56, 2.57, 2.58, 2.59, 2.6, 2.61, 2.62, 2.63, 2.64, 2.65, 2.66, 2.67, 2.68, 2.69, 2.7, 2.71, 2.72, 2.73, 2.74, 2.75, 2.76, 2.77, 2.78, 2.79, 2.8, 2.81, 2.82, 2.83, 2.84, 2.85, 2.86, 2.87, 2.88, 2.89, 2.9, 2.91, 2.92, 2.93, 2.94, 2.95, 2.96, 2.97, 2.98, 2.99, 3 μg/L.

In one embodiment the sample is isolated postsurgically at one day, two days, three days or after surgery more and determining a level of at least one biomarker comprises determining the level of PCT or fragment(s) thereof and a level of PCT or fragment(s) thereof above a threshold value±20% or more selected from a range of values in between 1 μg/L and 3 μg/L indicates an elevated likelihood of an infection, preferably a blood infection, and an adverse cardiovascular or cerebrovascular event, preferably a MACCE and/or a MINS. Any value within this range may be considered as an appropriate threshold value. For example, 1, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.291.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5, 1.51, 1.52, 1.53, 1.54, 1.55, 1.56, 1.57, 1.58, 1.59, 1.6, 1.61, 1.62, 1.63, 1.64, 1.65, 1.66, 1.67, 1.68, 1.69, 1.7, 1.71, 1.72, 1.73, 1.74, 1.75, 1.76, 1.77, 1.78, 1.79, 1.8, 1.81, 1.82, 1.83, 1.84, 1.85, 1.86, 1.87, 1.88, 1.89, 1.9, 1.91, 1.92, 1.93, 1.94, 1.95, 1.96, 1.97, 1.98, 1.99, 2, 2.01, 2.02, 2.03, 2.04, 2.05, 2.06, 2.07, 2.08, 2.09, 2.1, 2.11, 2.12, 2.13, 2.14, 2.15, 2.16, 2.17, 2.18, 2.19, 2.2, 2.21, 2.22, 2.23, 2.24, 2.25, 2.26, 2.27, 2.28, 2.29, 2.3, 2.31, 2.32, 2.33, 2.34, 2.35, 2.36, 2.37, 2.38, 2.39, 2.4, 2.41, 2.42, 2.43, 2.44, 2.45, 2.46, 2.47, 2.48, 2.49, 2.5, 2.51, 2.52, 2.53, 2.54, 2.55, 2.56, 2.57, 2.58, 2.59, 2.6, 2.61, 2.62, 2.63, 2.64, 2.65, 2.66, 2.67, 2.68, 2.69, 2.7, 2.71, 2.72, 2.73, 2.74, 2.75, 2.76, 2.77, 2.78, 2.79, 2.8, 2.81, 2.82, 2.83, 2.84, 2.85, 2.86, 2.87, 2.88, 2.89, 2.9, 2.91, 2.92, 2.93, 2.94, 2.95, 2.96, 2.97, 2.98, 2.99, 3 μg/L.

In one embodiment a first sample is isolated from the patient postsurgically at a first time point, preferably one day or more after surgery, and a second sample is isolated from the patient postsurgically at a subsequent second time point, wherein preferably between the first and second time point at least 12 hours, 24 hours, 36 hours, 48 hours or more have passed and determining a level of at least one biomarker comprises determining the level of PCT or fragment(s) thereof and an increase or a leveling in the level of PCT or fragment(s) thereof indicates an elevated likelihood of an infection, preferably a blood infection, and an adverse cardiovascular or cerebrovascular event, preferably a MACCE and/or a MINS.

In one embodiment a first sample is isolated from the patient postsurgically at a first time point, preferably one day or more after surgery, and a second sample is isolated from the patient postsurgically at a subsequent second time point, wherein preferably between the first and the second time point at least 12 hours, 24 hours, 36 hours, 48 hours or more have passed and determining a level of at least one biomarker comprises determining the level of PCT or fragment(s) thereof and an increase of the level of PCT or fragment(s) thereof above a threshold value±20% or more selected from a range of values in between 0.1 μg/L and 2 μg/L indicates an elevated likelihood of an infection, preferably a blood infection, and an adverse cardiovascular or cerebrovascular event, preferably a MACCE and/or a MINS. Any value within this range may be considered as an appropriate threshold value for the increase of PCT or fragment(s) in between the first and second time point. For example, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.291.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5, 1.51, 1.52, 1.53, 1.54, 1.55, 1.56, 1.57, 1.58, 1.59, 1.6, 1.61, 1.62, 1.63, 1.64, 1.65, 1.66, 1.67, 1.68, 1.69, 1.7, 1.71, 1.72, 1.73, 1.74, 1.75, 1.76, 1.77, 1.78, 1.79, 1.8, 1.81, 1.82, 1.83, 1.84, 1.85, 1.86, 1.87, 1.88, 1.89, 1.9, 1.91, 1.92, 1.93, 1.94, 1.95, 1.96, 1.97, 1.98, 1.99, 2 μg/L.

proET-1/Infection and MACCE/MINS:

In one embodiment determining a level of at least one biomarker comprises determining the level of proET-1 or fragment(s) thereof and a level of proET-1 or fragment(s) thereof above a threshold value indicates an elevated likelihood of an infection, preferably a blood infection, and an adverse cardiovascular or cerebrovascular event, preferably a MACCE and/or a MINS.

In one embodiment the sample is isolated preoperatively and determining a level of at least one biomarker comprises determining the level of proET-1 or fragment(s) thereof and a level of proET-1 or fragment(s) thereof above a threshold value±20% or more selected from a range of values in between 60 pmol/L and 85 pmol/L indicates an elevated likelihood of an infection, preferably a blood infection, and an adverse cardiovascular or cerebrovascular event, preferably a MACCE and/or a MINS. Any value within this range may be considered as an appropriate threshold value. For example, 60, 61, 62, 63, 64, 65, 66, 67, 68, 69, 70, 71, 72, 73, 74, 75, 76, 77, 78, 79, 80, 81, 82, 83, 84, 85 pmol/L.

In one embodiment the sample is isolated postsurgically within 12 hours, preferably within 6 hours after surgery and determining a level of at least one biomarker comprises determining the level of proET-1 or fragment(s) thereof and a level of proET-1 or fragment(s) thereof above a threshold value±20% or more selected from a range of values in between 80 pmol/L and 110 pmol/L indicates an elevated likelihood of an infection, preferably a blood infection, and an adverse cardiovascular or cerebrovascular event, preferably a MACCE and/or a MINS. Any value within this range may be considered as an appropriate threshold value. For example, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110 pmol/L.

In one embodiment the sample is isolated postsurgically one day after surgery and determining a level of at least one biomarker comprises determining the level of proET-1 or fragment(s) thereof and a level of proET-1 or fragment(s) thereof above a threshold value±20% or more selected from a range of values in between 80 pmol/L and 120 pmol/L indicates an elevated likelihood of an infection, preferably a blood infection, and an adverse cardiovascular or cerebrovascular event, preferably a MACCE and/or a MINS. Any value within this range may be considered as an appropriate threshold value. For example, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120 pmol/L.

In one embodiment the sample is isolated postsurgically two days after surgery and determining a level of at least one biomarker comprises determining the level of proET-1 or fragment(s) thereof and a level of proET-1 or fragment(s) thereof above a threshold value±20% or more selected from a range of values in between 80 pmol/L and 120 pmol/L indicates an elevated likelihood of an infection, preferably a blood infection, and an adverse cardiovascular or cerebrovascular event, preferably a MACCE and/or a MINS. Any value within this range may be considered as an appropriate threshold value. For example 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120 pmol/L.

In one embodiment the sample is isolated postsurgically three days after surgery and determining a level of at least one biomarker comprises determining the level of proET-1 or fragment(s) thereof and a level of proET-1 or fragment(s) thereof above a threshold value±20% or more selected from a range of values in between 80 pmol/L and 120 pmol/L indicates an elevated likelihood of an infection, preferably a blood infection, and an adverse cardiovascular or cerebrovascular event, preferably a MACCE and/or a MINS. Any value within this range may be considered as an appropriate threshold value. For example, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120 pmol/L.

In one embodiment the sample is isolated postsurgically and determining a level of at least one biomarker comprises determining the level of proET-1 or fragment(s) thereof and a level of proET-1 or fragment(s) thereof above a threshold value±20% or more selected from a range of values in between 80 pmol/L and 110 pmol/L indicates an elevated likelihood of an infection, preferably a blood infection, and an adverse cardiovascular or cerebrovascular event, preferably a MACCE and/or a MINS. Any value within this range may be considered as an appropriate threshold value. For example, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110 pmol/L.

In one embodiment the sample is isolated postsurgically at one day, two days, three days or more after surgery and determining a level of at least one biomarker comprises determining the level of proET-1 or fragment(s) thereof and a level of proET-1 or fragment(s) thereof above a threshold value±20% or more selected from a range of values in between 80 pmol/L and 120 pmol/L indicates an elevated likelihood of an infection, preferably a blood infection, and an adverse cardiovascular or cerebrovascular event, preferably a MACCE and/or a MINS. Any value within this range may be considered as an appropriate threshold value. For example, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120 pmol/L.

In one embodiment a first sample is isolated from the patient postsurgically at a first time point, preferably one day or more after surgery, and a second sample is isolated from the patient postsurgically at a subsequent second time point, wherein preferably between the first and the second time point at least 12 hours, 24 hours, 36 hours, 48 hours or more have passed and determining a level of at least one biomarker comprises determining the level of proET-1 or fragment(s) thereof and an increase or a leveling in the level of proET-1 or fragment(s) thereof indicates an elevated likelihood of an infection, preferably a blood infection, and an adverse cardiovascular or cerebrovascular event, preferably a MACCE and/or a MINS.

In one embodiment a first sample is isolated from the patient postsurgically at a first time point, preferably one day or more after surgery, and a second sample is isolated from the patient postsurgically at a subsequent second time point, wherein preferably between the first and the second time point at least 12 hours, 24 hours, 36 hours, 48 hours or more have passed and determining a level of at least one biomarker comprises determining the level of proET-1 or fragment(s) thereof and an increase of the level of proET-1 or fragment(s) thereof above a threshold value±20% or more selected from a range of values in between 1 pmol/L and 40 pmol/L indicates an elevated likelihood of an infection, preferably a blood infection, and an adverse cardiovascular or cerebrovascular event, preferably a MACCE and/or a MINS. Any value within this range may be considered as an appropriate threshold value for the increase of proET-1 or fragment(s) in between the first and second time point. For example, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 pmol/L.

### Embodiments Relating to Determining a Level of proET-1, PCT or proADM or Fragment(s) Thereof for Diagnosing or Predicting a Cardiovascular or Cerebrovascular Event, which is a PMI, and an Infection, Such as a Blood Infection

In one embodiment determining a level of at least one biomarker comprises determining the level of proET-1, PCT and/or proADM or fragment(s) thereof, and a level of proET-1, PCT and/or proADM or fragment(s) thereof above a threshold value indicates an elevated likelihood of an adverse cardiovascular or cerebrovascular event, preferably a perioperative myocardial injury (PMI), and the presence of an infection, preferably a blood infection. The sample may in some embodiments be obtained preoperatively or during surgery.

In a further embodiment, the present invention relates to a kit for carrying out the method of the present invention, wherein the kit comprises
detection reagents for determining the level of at least one biomarker selected from the group consisting of proADM, PCT and proET-1 or fragment(s) thereof, and
reference data for the likelihood of a postsurgical adverse event, in particular reference data for threshold or cut-off value(s), wherein said reference data is preferably stored on a computer readable medium and/or employed in the form of computer executable code configured for comparing the determined levels of the least one biomarker selected from the group consisting of proADM, PCT and proET-1 or fragment(s) thereof with the threshold or cut-off value(s).

### DETAILED DESCRIPTION OF THE INVENTION

The invention relates to a method for diagnosis, prognosis, risk assessment and/or risk stratification of an adverse event in the health of a postsurgical patient. As is evident from the data presented herein, the likelihood of a presence or absence of an adverse event in the health of a postsurgical patient is indicated by the level of proADM, PCT, proET-1 or fragment(s) thereof, which provides valuable information on therapy guidance, stratification and/or control.

The present invention has the following advantages over the conventional methods: the inventive methods and the kits are fast, objective, easy to use, reliable and precise for a diagnosis, prognosis, monitoring and therapy guidance of postsurgical patients.

The methods and kits of the invention relate to markers that are easily measurable in routine methods in hospitals, because the levels of proADM, PCT and/or proET-1 can be determined in routinely obtained blood samples or further biological fluids or samples obtained from a subject.

As used herein, the “patient” or “subject” may be a vertebrate. In the context of the present invention, the term “subject” includes both humans and animals, particularly mammals, and other organisms.

A “postsurgical patient” refers to a patient or subject that has undergone surgery.

As used herein, the term “surgery” refers to the treatment of diseases, injuries, and deformities by manual or operative methods. Common surgical procedures include, but are not limited to, abdominal, aural, bench, cardiac, cineplastic, conservative, cosmetic, cytoreductive, dental, dentofacial, general, major, minor, Moh's, open heart, organ transplantation, orthopedic, plastic, psychiatric, radical, reconstructive, sonic, stereotactic, structural, thoracic, and veterinary surgery. The method of the present invention is suitable for patients that are to undergo any type of surgery dealing with any portion of the body, including but not limited to those described above, as well as any type of any general, major or minor surgery.

Minor surgery is any invasive operative procedure in which only skin or mucus membranes and connective tissue is resected e.g. vascular cutdown for catheter placement, implanting pumps in subcutaneous tissue.

A major surgery is any invasive operative procedure in which a more extensive resection is performed, e.g. a body cavity is entered, organs are removed, or normal anatomy is altered.

Preferably a major surgery has to be performed under general anesthesia and require at least one overnight stay.

A “noncardiac surgery” refers to any surgery that is not a cardiac surgery.

A “cardiac surgery” refers to any surgical procedure performed on the heart or surrounding tissue (e.g., cardiac tissue, nerve tissue, vascular tissue, and/or the like). Cardiac surgeries include open heart surgery, heart valve surgery, comprising pericardium open surgery (e.g., within the pericardium is selected via thoracoscopy, thoracotomy, mediastinotomy, sternum incision, cut and opened by the diaphragm xiphoid route scheme), bypass surgery (e.g., coronary artery bypass surgery) or aneurysm repair.

A “major noncardiac surgery” encompasses preferably a major abdominal surgery, a gastrointestinal, a respiratory surgery, a thoracic surgery, an endocrine surgery, a lymphatic surgery, a bone or joint surgery, a surgery of the genital part and/or a breast surgery.

An “abdominal surgery” covers preferably any surgical procedures that involve opening the abdomen of a patient.

A “gastrointestinal surgery” or an “abdominal surgery” encompasses for instance preferably an appendectomy, a caesarean section, an exploratory laparotomy, a glossectomy, a esophagectomy, a gastrectomy, a proctocolectomy, colectomy, a hepatectomy, a cholecystectomy, a pancreatectomy, a gastrostomy, a gastroduodenostomy, a gastroenterostomy, an illeostomy, a jejunostomy, a colostomy, a cholecystostomy, a hepatoportoenterostomy, a myotomy or a sphincterotomy

As used herein, the term “preoperative” is used synonymously with “presurgical” and refers to a period before surgery. Preferably the “preoperative” period begins preferably with the scheduling of the surgery upon and may include the time period preparing the subject for surgery and ends with the beginning of manual or operative methods that constitute the surgery.

As used herein, the term “perioperative” refers to the time period during a surgical operation, i.e. the time period during which the manual or operative methods that constitute the surgery are carried out.

As used herein, the term “postsurgical” is used synonymously with “postoperative” and refers to the time period after a surgical operation and begins with the ending of the manual or operative methods that constitute the surgery and the subsequent days or weeks after a surgery.

When a biomarker is determined preoperatively as described herein, preoperatively preferably refers to a time point within 7 days, preferably within 3 days, within one day or less before the surgery. It is preferred that in these cases preoperatively refers to at least 6 hours, more preferably at least 3 hours before the surgery.

When a biomarker is determined postsurgically as described herein, postsurgical preferably refers to a time point within 14 days, preferably 10 days, 7 days, 5 days, most preferably three days after the surgery.

As used herein, an isolation of a sample, while the patient is monitored on the post anastasia care unit (PACU), preferably refers to an isolation of a sample postsurgically within 12 hours, preferably within 6 hours after surgery.

As used herein, an isolation of a sample one day after surgery (POD1) preferably refers to an isolation of a sample postsurgically in between 12 hours and 36 hours after surgery, preferably in between 18 hours and 30 hours after surgery, most preferably approximately 24 hours after surgery.

As used herein, an isolation of a sample two days after surgery (POD2) preferably refers to an isolation of a sample postsurgically in between 36 hours and 60 hours after surgery, preferably in between 42 hours and 54 hours after surgery, most preferably approximately 48 hours after surgery.

As used herein, an isolation of a sample three days after surgery (POD3) preferably refers to an isolation of a sample postsurgically in between 60 hours and 84 hours after surgery, preferably in between 66 hours and 78 hours after surgery, most preferably approximately 72 hours after surgery.

When a postsurgical adverse event is predicted or diagnosed using the method described herein, the time period for the occurrence of the postsurgical adverse event encompasses preferably 60 days after surgery, more preferably 30 days after surgery.

As used herein, the term “medical procedure” refers to any clinical or diagnostic procedure performed by a medical practitioner (e.g., including, but not limited to a physician or physician's assistant, a nurse or nurse practitioner, or a veterinarian).

In the context of the present invention, an “adverse event in the health of a postsurgical patient” relates to events that indicate complications or worsening of the health state of a patient after having undergone surgery. Such adverse events include, without limitation, a cardiac event, a cardiovascular event, a cerebrovascular event, occurrence of an infection or a new infection, a sepsis-like systemic infection, a sepsis, an organ failure and deterioration of the patient's general clinical signs or symptoms, such as hypotension or hypertension, tachycardia or bradycardia, death of the patient or death of a patient within 30 days after surgery.

Furthermore, examples of adverse events include situations where a deterioration of clinical symptoms indicates the requirement for therapeutic measures, such as a focus cleaning procedure, antimicrobial treatment, antibiotic treatment, transfusion of blood products, infusion of colloids, invasive mechanical ventilation, non-invasive mechanical ventilation, emergency surgery, organ replacement therapy, such as renal or liver replacement, and vasopressor therapy

As used herein, “diagnosis” in the context of the present invention relates to the recognition and (early) detection of a clinical condition. Also, the assessment of the severity may be encompassed by the term “diagnosis”.

“Prognosis” relates to the prediction of an outcome or a risk for a subject. This may also include an estimation of the chance of recovery or the chance of an adverse outcome for said subject.

The method described herein comprises the determination of a level of at least one biomarker which correlates with the likelihood of an adverse event in the health of a postsurgical patient. This preferably means that the likelihood of the occurrence of an adverse event can be assessed on the comparison of the level of one of the biomarkers to a reference level (such as a threshold or cut-off value and/or a population average), wherein the reference level may correspond the level of a biomarker in patients who have not and will not develop a postsurgical adverse event.

Accordingly, the determination of the biomarkers as described herein may be used to assess whether the patient has an elevated or decreased likelihood for an adverse event after surgery.

The method comprises a diagnosis as well as a prognosis. A level of biomarkers that indicates an elevated likelihood of an adverse event may thus indicate a high likelihood that that the patient has suffered of said adverse event postsurgically at the time of sample isolation or the patient will suffer of an adverse event after sample isolation.

In terms of an early diagnosis or future prognosis of a postsurgical adverse event the method is particular suited for a risk assessment or stratification. This means, that the method of the invention can discriminate high risk patients, who are more likely to suffer from (further) complications, or whose state will become more critical in the future, from low risk patients, whose health state is stable or even improving, so that it is not expected that they have suffered of an adverse event or will suffer from an adverse event, which might require certain therapeutic measures.

In the present invention, the terms “risk assessment” and “risk stratification” relate to the grouping of subjects into different risk groups according to their further prognosis. Risk assessment also relates to stratification for applying preventive and/or therapeutic measures. The term “therapy stratification” in particular relates to grouping or classifying patients into different groups, such as risk groups or therapy groups that receive certain differential therapeutic measures depending on their classification. The term “therapy stratification” also relates to grouping or classifying patients with infections or having symptoms of an infectious disease into a group that are not in need to receive certain therapeutic measures.

The methods of the invention may also be used for monitoring, therapy monitoring, therapy guidance and/or therapy control. “Monitoring” relates to keeping track of a patient and potentially occurring complications, e.g. to analyze the progression of the healing process or the influence of a particular treatment or therapy on the health state of the patient.

The term “therapy monitoring” or “therapy control” in the context of the present invention refers to the monitoring and/or adjustment of a therapeutic treatment of said patient, for example by obtaining feedback on the efficacy of the therapy. As used herein, the term “therapy guidance” refers to application of certain therapies, therapeutic actions or medical interventions based on the value/level of one or more biomarkers and/or clinical parameter and/or clinical scores. This includes the adjustment of a therapy or the discontinuation of a therapy.

The method described herein relates to the diagnosis, prognosis, risk assessment and/or risk stratification of an adverse event in the health of a postsurgical patient based on a determination of the level of at least one biomarker selected from the group consisting of proADM, PCT and proET-1.

The expression “at least one biomarker selected from the group consisting of proADM, PCT and proET-1” as well as the expression “proADM, PCT and/or proET-1” are preferably to be understood to encompass all possible combinations including the determination of exactly one of said three biomarker, i.e. proADM, PCT or pro ET-1 as well as a determination of exactly two of said biomarkers, i.e. a combination of proADM and PCT, proADM and proET-1 or PCT and proET-1, or a determination of all three biomarkers proADM, PCT and proET-1.

It is further to be understood that said expressions do not exclude the additional determination of further biomarkers or parameters.

It is understood that in the context of the present invention “determining the level of proADM or fragment(s) thereof” or the like refers to any means of determining proADM or a fragment thereof. The fragment can have any length, e.g. at least about 5, 10, 20, 30, 40, 50 or 100 amino acids, so long as the fragment allows the unambiguous determination of the level of proADM or fragment thereof. In particular preferred aspects of the invention, “determining the level of proADM” refers to determining the level of midregional proadrenomedullin (MR-proADM). MR-proADM is a fragment and/or region of proADM.

The peptide adrenomedullin (ADM) was discovered as a hypotensive peptide comprising 52 amino acids, which had been isolated from a human phenochromocytome (Kitamura et al., 1993). Adrenomedullin (ADM) is encoded as a precursor peptide comprising 185 amino acids (“preproadrenomedullin” or “pre proADM”). An exemplary amino acid sequence of pre-pro-ADM is given in SEQ ID NO: 1.

| SEQ ID NO: 1: amino acid sequence of pre-pro-ADM: | |
|---|---|
| 1 | MKLVSVALMY LGSLAFLGAD TARLDVASEF RKKWNKWALS RGKRELRMSS |
| 51 | SYPTGLADVK AGPAQTLIRP QDMKGASRSP EDSSPDAARI RVKRYRQSMN |
| 101 | NFQGLRSFGC RFGTCTVQKL AHQIYQFTDK DKDNVAPRSK ISPQGYGRRR |
| 151 | RRSLPEAGPG RTLVSSKPQA HGAPAPPSGS APHFL |

ADM comprises the positions 95-146 of the pre-proADM amino acid sequence and is a splice product thereof. “Proadrenomedullin” (“proADM”) refers to pre-proADM without the signal sequence (amino acids 1 to 21), i.e. to amino acid residues 22 to 185 of pre-proADM. “Midregional proadrenomedullin” (“MR-proADM”) refers to the amino acids 42 to 95 of pre-proADM. An exemplary amino acid sequence of MR-proADM is given in SEQ ID NO: 2.

SEQ ID NO: 2: amino acid sequence of MR-pro-ADM
(AS 45-92 of pre-pro-ADM):
ELRMSSSYPT GLADVKAGPA QTLIRPQDMK GASRSPEDSS
PDAARIRV

It is also envisaged herein that a peptide and fragment thereof of pre-proADM or MR-proADM can be used for the herein described methods. For example, the peptide or the fragment thereof can comprise the amino acids 22-41 of pre-proADM (PAMP peptide) or amino acids 95-146 of pre-proADM (mature adrenomedullin, including the biologically active form, also known as bio-ADM). A C-terminal fragment of proADM (amino acids 153 to 185 of pre proADM) is called adrenotensin. Fragments of the proADM peptides or fragments of the MR-proADM can comprise, for example, at least about 5, 10, 20, 30 or more amino acids. Accordingly, the fragment of proADM may, for example, be selected from the group consisting of MR-proADM, PAMP, adrenotensin and mature adrenomedullin, preferably herein the fragment is MR-proADM.

The determination of these various forms of ADM or proADM and fragments thereof also encompass measuring and/or detecting specific sub-regions of these molecules, for example by employing antibodies or other affinity reagents directed against a particular portion of the molecules, or by determining the presence and/or quantity of the molecules by measuring a portion of the protein using mass spectrometry. Any one or more of the “ADM peptides or fragments” described herein may be employed in the present invention.

The level of proADM in the sample of the subject can be determined by immunoassays as described herein. As used herein, the level of ribonucleic acid or deoxyribonucleic acids encoding “proAdrenomedullin” or “proADM” can also be determined. Methods for the determination of proADM are known to a skilled person, for example by using products obtained from Thermo Fisher Scientific/B⋅R⋅A⋅H⋅M⋅S GmbH.

It is understood that in the context of the present invention “determining the level of proEndothelin-1 (proET-1) or fragment(s) thereof” or the like refers to determining proEndothelin-1 or a fragment thereof. The fragment can have any length, e.g. at least about 5, 10, 20, 30, 40, 50 or 100 amino acids, so long as the fragment allows the unambiguous determination of the level of the Endothelin-1.

Endothelin-1 is a member of a family of 21-amino acid peptides and is considered having an important pathophysiological role in cardiovascular dysfunction (Haaf et al. 2014, Papassotiriou et al. 2014) The gene of ET-1 is found on chromosome 6 and its expression is regulated by several factors, e.g. angiotensin II, cytokines, hypoxia, thrombin, free radicals, endotoxins, and shear stress (Zhang et al. 2017, Bhandari et al. 2014). It is primarily synthesized by vascular endothelial cells but also in smooth muscle cells, cardiomyocytes, and cardiac fibroblast cells as well as in various tissues in the body. ET-1 is initially synthesized as the precursor preproET-1. Cleavage of preproET-1 yields proET-1 which is further cleaved by furin generating two biologically inactive precursors named big-1 and C-terminal of proET-1 (CT-proET-1). BigET-1 is finally converted by metalloproteinase endothelin converting enzyme (ECE-1) generating ET-1 (Zhang et al. 2017).

As used herein, “proEndothelin 1” or “proET-1” relates to a peptide spanning amino acid residues 18 to 212, or fragments thereof, of the amino acid precursor peptide of Endothelin-(pre-pro-Endothelin-1). An exemplary sequence of the 212 amino acid precursor peptide of Endothelin-(pre-pro-Endothelin-1) is given in SEQ ID NO:3. An exemplary amino acid sequence of proET-1 is given in SEQ ID NO:4. ProET-1 is cleaved into mature ET-1, big-ET-land C-terminal proET-1 (CT-proET-1). ET-1 relates to the amino acid residues 53 to 73 of pre-pro-ET-1. CT-proET-1 relates to amino acid residues 168 to 212 of pre-pro-ET-1. An exemplary amino acid sequence of CT-pro ET-1 is provided in SEQ ID NO:4. Big-ET-1 comprises the amino acid residues 53 to 90 of pre-pro-ET-1.

| SEQ ID NO: 3: amino acid sequence of pre-pro-ETI: | |
|---|---|
| 1 | MDYLLMIFSL LFVACQGAPE TAVLGAELSA VGENGGEKPT PSPPWRLRRS |
| 51 | KRCSCSSLMD KECVYFCHLD IIWVNTPEHV VPYGLGSPRS KRALENLLPT |
| 101 | KATDRENRCQ CASQKDKKCW NFCQAGKELR AEDIMEKDWN NHKKGKDCSK |
| 151 | LGKKCIYQQL VRGRKIRRSS EEHLRQTRSE TMRNSVKSSF HDPKLKGKPS |
| 201 | RERYVTHNRA HW |
| SEQ ID NO: 4: amino acid sequence of pro-ETI | |
| 1 | APETAVLGAE LSAVGENGGE KPTPSPPWRL RRSKRCSCSS LMDKECVYFC |
| 51 | HLDIIWVNTP EHVVPYGLGS PRSKRALENL LPTKATDREN RCQCASQKDK |
| 101 | KCWNFCQAGK ELRAEDIMEK DWNNHKKGKD CSKLGKKCIY QQLVRGRKIR |
| 151 | RSSEEHLRQT RSETMRNSVK SSFHDPKLKG KPSRERYVTH NRAHW |
| SEQ ID NO: 5: amino acid sequence of CT-proET-1 | |
| 1 | RSSEEHLRQT RSETMRNSVK SSFHDPKLKG KPSRERYVTH NRAHW |

In particular preferred embodiment CT-proET-1 is measured to determine the level of proEndothelin-1. The length of proEndothelin-1 fragments is therefore preferably at least 12 amino acids, preferably more than 20 amino acids, more preferably more than 40 amino acids.

The determination of these various forms of CT-proET-1 or proET1 and fragments thereof also encompass measuring and/or detecting specific sub-regions of these molecules, for example by employing antibodies or other affinity reagents directed against a particular portion of the molecules, or by determining the presence and/or quantity of the molecules by measuring a portion of the protein using mass spectrometry. Any one or more of the “ET-1 peptides or fragments” described herein may be employed in the present invention.

The level of proET-1 in the sample of the subject can be determined by immunoassays as described herein. As used herein, the level of ribonucleic acid or deoxyribonucleic acids encoding “proEndothelin-1” or “proET-1” can also be determined. Methods for the determination of proET-1 are known to a skilled person, for example according to EP1564558.

It is understood that in the context of the present invention “determining the level of procalcitonin or fragment(s) thereof”, “determining the level of PCT or fragment(s) thereof” or the like refers to determining procalcitonin or a fragment thereof. The fragment can have any length, e.g. at least about 5, 10, 20, 30, 40, 50 or 100 amino acids, so long as the fragment allows the unambiguous determination of the level of the procalcitonin.

As used herein, “procalcitonin” or “PCT” relates to a peptide spanning amino acid residues 1-116, 2-116, 3-116, or fragments thereof, of the procalcitonin peptide. PCT is a peptide precursor of the hormone calcitonin. An exemplary sequence of 1-116 procalcitonin peptide is given in SEQ ID NO:6

| SEQ ID NO: 6: amino acid sequence of 1-116 PCT | |
|---|---|
| 1 | APFRSALESS PADPATLSED EARLLLAALV QDYVQMKASE LEQEQEREGS |
| 51 | SLDSPRSKRC GNLSTCMLGT YTQDFNKFHT FPQTAIGVGA PGKKRDMSSD |
| 101 | LERDHRPHVS MPQNAN |

Thus the length of procalcitonin fragments is at least 12 amino acids, preferably more than 50 amino acids, more preferably more than 110 amino acids. Determination of PCT may comprise determination of N-terminal PCT (SEQ ID NO:7), calcitonin (SEQ ID NO:8) or katacalcin (SEQ ID NO:9) and may also comprise post-translational modifications such as glycosylation, liposidation or derivatisation. Procalcitonin is a precursor of calcitonin and katacalcin. Thus, under normal conditions the PCT levels in the circulation are very low (<about 0.05 ng/ml).

| SEQ ID NO: 7 (AS 1-57 of PCT; amino acid sequence of N-terminal PCT): | |
|---|---|
| 1 | APFRSALESS PADPATLSED EARLLLAALV QDYVQMKASE LEQEQEREGS |
| 51 | SLDSPRS |
| SEQ ID NO: 8 (amino acid sequence of Calcitonin, AS 60-91 of PCT): | |
| 60 | CGNLSTCMLG TYTQDFNKFH TFPQTAIGVG AP |
| SEQ ID NO: 9 (amino acid sequence of Katacalcin, AS 96-116 of PCT): | |
| 96 | DMSSDLERDH RPHVSMPQNA N |

The level of PCT in the sample of the subject can be determined by immunoassays as described herein. As used herein, the level of ribonucleic acid or deoxyribonucleic acids encoding “procalcitonin” or “PCT” can also be determined. Methods for the determination of PCT are known to a skilled person, for example by using products obtained from Thermo Fisher Scientific/B⋅R⋅A⋅H⋅M⋅S GmbH.

The determination of procalcitonin and fragments thereof also encompass measuring and/or detecting specific sub-regions of these molecules, for example by employing antibodies or other affinity reagents directed against a particular portion of the molecules, or by determining the presence and/or quantity of the molecules by measuring a portion of the protein using mass spectrometry.

Accordingly, the methods and kits of the present invention can also comprise determining at least one further biomarker, marker, clinical score and/or parameter in addition to proADM, PCT and/or proET-1.

As used herein, a parameter is a characteristic, feature, or measurable factor that can help in defining a particular system. A parameter is an important element for health- and physiology-related assessments, such as a disease/disorder/clinical condition risk, preferably organ dysfunction(s). Furthermore, a parameter is defined as a characteristic that is objectively measured and evaluated as an indicator of normal biological processes, pathogenic processes, or pharmacologic responses to a therapeutic intervention. An exemplary parameter can be selected from the group consisting of Acute Physiology and Chronic Health Evaluation II (APACHE II), the simplified acute physiology score (SAPSII score), sequential organ failure assessment score (SOFA score), quick sequential organ failure assessment score (qSOFA), body mass index, weight, age, sex, IGS II, liquid intake, white blood cell count, sodium, potassium, temperature, blood pressure, dopamine, bilirubin, respiratory rate, partial pressure of oxygen, World Federation of Neurosurgical Societies (WFNS) grading, and Glasgow Coma Scale (GCS).

As used herein, terms such as “marker”, “surrogate”, “prognostic marker”, “factor” or “biomarker” or “biological marker” are used interchangeably and relate to measurable and quantifiable biological markers (e.g., specific protein or enzyme concentration or a fragment thereof, specific hormone concentration or a fragment thereof, or presence of biological substances or a fragment thereof) which serve as indices for health- and physiology-related assessments, such as a disease/disorder/clinical condition risk, preferably an adverse event. A marker or biomarker is defined as a characteristic that can be objectively measured and evaluated as an indicator of normal biological processes, pathogenic processes, or pharmacologic responses to a therapeutic intervention. Biomarkers may be measured in a sample (as a blood, plasma, urine, or tissue test).

The at least one further marker and/or parameter of said subject can be selected from the group consisting of a level of lactate in said sample, the sequential organ failure assessment score (SOFA score) of said subject, the simplified acute physiology score (SAPSII) of said subject, the Acute Physiology and Chronic Health Evaluation II (APACHE II) score of said subject, a American Society of Anesthesiologists Physical Status (ASAPS) and a level of the soluble fms-like tyrosine kinase-1 (sFlt-1), Histone H2A, Histone H2B, Histone H3, Histone H4, Arginine Vasopressin (AVP), Atrial Natriuretic Peptide (ANP), Myoglobin, Creatin Kinase Neutrophil Gelatinase-Associated Lipocalin (NGAL), Troponin, Cardiac troponin T (cnTNT), Brain Natriuretic Peptide (BNP), C-Reactive Protein (CRP), Pancreatic Stone Protein (PSP), Triggering Receptor Expressed on Myeloid Cells 1 (TREM1), Interleukin-6 (IL-6), Interleukin-1, Interleukin-24 (IL-24), Interleukin-22 (IL-22), Interleukin (IL-20) other ILs, Presepsin (sCD14-ST), Lipopolysaccharide Binding Protein (LBP), Alpha-1-Antitrypsin, Matrix Metalloproteinase 2 (MMP2), Metalloproteinase 2 (MMP8), Matrix Metalloproteinase 9 (MMP9), Matrix Metalloproteinase 7 (MMP7, Placental growth factor (PIGF), Chromogranin A, S100A protein, S100B protein and Tumor Necrosis Factor α (TNFα), Neopterin, Alpha-1-Antitrypsin, pro-arginine vasopressin (AVP, proAVP or Copeptin), atrial natriuretic peptide (ANP, pro-ANP), Endothelin-1, CCL1/TCA3, CCL11, CCL12/MCP-5, CCL13/MCP-4, CCL14, CCL15, CCL16, CCL17/TARC, CCL18, CCL19, CCL2/MCP-1, CCL20, CCL21, CCL22/MDC, CCL23, CCL24, CCL25, CCL26, CCL27, CCL28, CCL3, CCL3L3, CCL4, CCL4L1/LAG-1, CCL5, CCL6, CCL7, CCL8, CCL9, CX3CL1, CXCL1, CXCL10, CXCL11, CXCL12, CXCL13, CXCL14, CXCL15, CXCL16, CXCL17, CXCL2/MIP-2, CXCL3, CXCL4, CXCL5, CXCL6, CXCL7/Ppbp, CXCL9, IL8/CXCL8, XCL1, XCL2, FAM19A1, FAM19A2, FAM19A3, FAM19A4, FAM19A5, CLCF1, CNTF, IL11, IL31, IL6, Leptin, LIF, OSM, IFNA1, IFNA10, IFNA13, IFNA14, IFNA2, IFNA4, IFNA7, IFNB1, IFNE, IFNG, IFNZ, IFNA8, IFNA5/IFNaG, IFNω/IFNW1, BAFF, 4-1BBL, TNFSF8, CD40LG, CD70, CD95L/CD178, EDA-A1, TNFSF14, LTA/TNFB, LTB, TNFa, TNFSF10, TNFSF11, TNFSF12, TNFSF13, TNFSF15, TNFSF4, IL18, IL18BP, IL1A, IL1B, IL1F10, IL1F3/IL1RA, IL1F5, IL1F6, IL1F7, IL1F8, IL1RL2, IL1F9, IL33 or a fragment thereof.

As used herein, the “sequential organ failure assessment score” or “SOFA score” is one score used to track a patient's status during the stay in an intensive care unit (ICU). The SOFA score is a scoring system to determine the extent of a person's organ function or rate of failure. The score is based on six different scores, one each for the respiratory, cardiovascular, hepatic, coagulation, renal and neurological systems. Both the mean and highest SOFA scores being predictors of outcome. An increase in SOFA score during the first 24 to 48 hours in the ICU predicts a mortality rate of at least 50% up to 95%. Scores less than 9 give predictive mortality at 33% while above 14 can be close to or above 95%.

As used herein, the quick SOFA score (qSOFA) is a scoring system that indicates a patient's organ dysfunction or mortality risk. The score is based on three criteria: 1) an alteration in mental status, 2) a decrease in systolic blood pressure of less than 100 mm Hg, 3) a respiration rate greater than 22 breaths per minute. Patients with two or more of these conditions are at greater risk of having an organ dysfunction or to die.

As used herein, “APACHE II” or “Acute Physiology and Chronic Health Evaluation II” is a severity-of-disease classification scoring system (Knaus et al., 1985). It can be applied within 24 hours of admission of a patient to an intensive care unit (ICU) and may be determined based on 12 different physiologic parameters: AaDO2 or PaO2 (depending on FiO2), temperature (rectal), mean arterial pressure, pH arterial, heart rate, respiratory rate, sodium (serum), potassium (serum), creatinine, hematocrit, white blood cell count and Glasgow Coma Scale.

As used herein, “SAPS II” or “Simplified Acute Physiology Score II” relates to a system for classifying the severity of a disease or disorder (see Le Gall J R et al., A new Simplified Acute Physiology Score (SAPS II) based on a European/North American multicenter study. JAMA. 1993; 270(24):2957-63.). The SAPS II score is made of 12 physiological variables and 3 disease-related variables. The point score is calculated from 12 routine physiological measurements, information about previous health status and some information obtained at admission to the ICU. The SAPS II score can be determined at any time, preferably, at day 2. The “worst” measurement is defined as the measure that correlates to the highest number of points. The SAPS II score ranges from 0 to 163 points. The classification system includes the followings parameters: Age, Heart Rate, Systolic Blood Pressure, Temperature, Glasgow Coma Scale, Mechanical Ventilation or CPAP, PaO2, FiO2, Urine Output, Blood Urea Nitrogen, Sodium, Potassium, Bicarbonate, Bilirubin, White Blood Cell, Chronic diseases and Type of admission. There is a sigmoidal relationship between mortality and the total SAPS II score. The mortality of a subject is 10% at a SAPSII score of 29 points, the mortality is 25% at a SAPSII score of 40 points, the mortality is 50% at a SAPSII score of 52 points, the mortality is 75% at a SAPSII score of 64 points, the mortality is 90% at a SAPSII score of 77 points (Le Gall loc. cit.).

As used herein “ASA”, “ASA physical status, “ASAPS” or “ASA score” is used to refer to the physical status classification system developed by American Society of Anesthesiologists (ASA) to offer clinicians a simple categorization of a patient's physiological status that can be helpful in predicting operative risk. Preferably the ASAPS comprises 6 classes of which indicate the increasing operative (Doyle et al. 2018):

“ASA I:” A normal healthy patient. Example: Fit, nonobese (BMI under 30), a nonsmoking patient with good exercise tolerance. “ASA II:” A patient with a mild systemic disease. Example: Patient with no functional limitations and a well-controlled disease (e.g., treated hypertension, obesity with BMI under 35, frequent social drinker or is a cigarette smoker). “ASA III”: A patient with a severe systemic disease that is not life-threatening. Example: Patient with some functional limitation as a result of disease (e.g., poorly treated hypertension or diabetes, morbid obesity, chronic renal failure, a bronchospastic disease with intermittent exacerbation, stable angina, implanted pacemaker). “ASA IV”: A patient with a severe systemic disease that is a constant threat to life. Example: Patient with functional limitation from severe, life-threatening disease (e.g., unstable angina, poorly controlled COPD, symptomatic CHF, recent (less than three months ago) myocardial infarction or stroke. “ASA V”: A moribund patient who is not expected to survive without the operation. The patient is not expected to survive beyond the next 24 hours without surgery. Examples: ruptured abdominal aortic aneurysm, massive trauma, and extensive intracranial hemorrhage with mass effect. “ASA VI”: A brain-dead patient whose organs are being removed with the intention of transplanting them into another patient.

As used herein, the term “sample” is a biological sample that is obtained or isolated from the patient or subject. “Sample” as used herein may, e.g., refer to a sample of bodily fluid or tissue obtained for the purpose of diagnosis, prognosis, or evaluation of a subject of interest, such as a patient. Preferably herein, the sample is a sample of a bodily fluid, such as blood, serum, plasma, cerebrospinal fluid, urine, saliva, sputum, pleural effusions, cells, a cellular extract, a tissue sample, a tissue biopsy, a stool sample and the like. Particularly, the sample is blood, blood plasma, blood serum, or urine.

Embodiments of the present invention refer to the isolation of a first sample and the isolation of a second sample. In the context of the method of the present invention, the terms “first sample” and “second sample” relate to the relative determination of the temporal order of isolation of the samples employed in the method of the present invention. When the terms first sample and second sample are used in specifying the present method, these samples are not to be considered as absolute determinations of the number of samples taken. Therefore, additional samples may be isolated from the patient before, during or after isolation of the first and/or the second sample, or between the first or second samples, wherein these additional samples may or may not be used in the method of the present invention. The first sample may therefore be considered as any previously obtained sample. The second sample may be considered as any further or subsequent sample.

“Plasma” in the context of the present invention is the virtually cell-free supernatant of blood containing anticoagulant obtained after centrifugation. Exemplary anticoagulants include calcium ion binding compounds such as EDTA or citrate and thrombin inhibitors such as heparinates or hirudin. Cell-free plasma can be obtained by centrifugation of the anticoagulated blood (e.g. citrated, EDTA or heparinized blood), for example for at least 15 minutes at 2000 to 3000 g.

“Serum” in the context of the present invention is the liquid fraction of whole blood that is collected after the blood is allowed to clot. When coagulated blood (clotted blood) is centrifuged serum can be obtained as supernatant.

As used herein, “urine” is a liquid product of the body secreted by the kidneys through a process called urination (or micturition) and excreted through the urethra.

In preferred embodiments of the present invention the method may be used for diagnosis, prognosis, risk assessment and/or risk stratification of an infection, preferably a blood infection, a respiratory tract infection, a urinary tract infection, a skin infection, more preferably a blood stream infection, a sepsis, severe sepsis and/or septic shock.

As used herein, “infection” within the scope of the invention means a pathological process caused by the invasion of normally sterile tissue or fluid by pathogenic or potentially pathogenic agents/pathogens, organisms and/or microorganisms, and relates preferably to infection(s) by bacteria, viruses, fungi, and/or parasites. Accordingly, the infection can be a bacterial infection, viral infection, and/or fungal infection. The infection can be a local or systemic infection. For the purposes of the invention, a viral infection may be considered as infection by a microorganism.

Further, the subject suffering from an infection can suffer from more than one source(s) of infection simultaneously. For example, the subject suffering from an infection can suffer from a bacterial infection and viral infection; from a viral infection and fungal infection; from a bacterial and fungal infection, and from a bacterial infection, fungal infection and viral infection, or suffer from a mixed infection comprising one or more of the infections listed herein, including potentially a superinfection, for example one or more bacterial infections in addition to one or more viral infections and/or one or more fungal infections

As used herein “infectious disease” comprises all diseases or disorders that are associated with bacterial and/or viral and/or fungal infections.

In one embodiment the infection to be detected or to be tested for may be selected from species of Bordetella, such as Bordetella pertussis, Borrelia, such as Borrelia burgdorferi, Brucella, such as Brucella abortus, Brucella canis, Brucella melitensis or Brucella suis, Campylobacter, such as Campylobacter jejuni, Chlamydia and Chlamydophila, such as Chlamydia pneumonia, Chlamydia trachomatis, Chlamydophila psittaci, Clostridium, such as Clostridium botulinum, Clostridium difficile, Clostridium perfringens, Clostridium tetani, Corynebacterium, such as Corynebacterium diphtheria, Enterococcus, such as Enterococcus faecalis, Enterococcus faecium, Escherichia, such as Escherichia coli, Francisella, such as Francisella tularensis, Haemophilus, such as Haemophilus influenza, Helicobacter, such as Helicobacter pylori, Legionella, such as Legionella pneumophila, Leptospira, such as Leptospira interrogans, Listeria, such as Listeria monocytogenes, Mycobacterium, such as Mycobacterium leprae, Mycobacterium tuberculosis, Mycobacterium ulcerans, Mycoplasma, such as Mycoplasma pneumonia, Neisseria, such as Neisseria gonorrhoeae, Neisseria meningitides, Pseudomonas, such as Pseudomonas aeruginosa, Rickettsia, such as Rickettsia rickettsia, Salmonella, such as Salmonella typhi, Salmonella typhimurium, Shigella, such as Shigella sonnei, Staphylococcus, such as Staphylococcus aureus, Staphylococcus epidermidis, Staphylococcus saprophyticus, Streptococcus, such as Streptococcus agalactiae, Streptococcus pneumonia, Streptococcus pyogenes, Treponema, such as Treponema pallidum, Vibrio, such as Vibrio cholera, Yersinia, such as Yersinia pestis, Yersinia enterocolitica or Yersinia pseudotuberculosis.

Pathogenic fungi are fungi that cause disease in humans or other organisms. Candida species are important human pathogens that are best known for causing opportunist infections in immunocompromised hosts (e.g. transplant patients, AIDS sufferers, cancer patients). Infections are difficult to treat and can be very serious: 30-40% of systemic infections result in death. Aspergillosis is another potential fungal pathogen. Aspergillus can cause disease in three major ways: through the production of mycotoxins; through induction of allergenic responses; and through localized or systemic infections. With the latter two categories, the immune status of the host is pivotal. The most common pathogenic species are Aspergillus fumigatus and Aspergillus flavus. Aspergillus flavus produces aflatoxin which is both a toxin and a carcinogen and which can potentially contaminate foods. Aspergillus fumigatus and Aspergillus clavatus can cause disease. Cryptococcus neoformans can cause disease in humans. Cryptococcus neoformans is the major human and animal pathogen. Cryptococcus laurentii and Cryptococcus albidus have been known to occasionally cause moderate-to-severe disease in human patients with compromised immunity. Cryptococcus gattii is endemic to tropical parts of the continent of Africa and Australia and can cause disease. Histoplasma capsulatum can cause histoplasmosis in humans, dogs and cats. Pneumocystis jirovecii (or Pneumocystis carinii) can cause a form of pneumonia in people with weakened immune systems, such as premature children, the elderly, and AIDS patients. Stachybotrys chartarum or “black mould” can cause respiratory damage and severe headaches.

In one embodiment the infection to be detected or to be tested for may be selected from Acinetobacter baumannii, Klebsiella pneumoniae, Acinetobacter lwoffii, Listeria monocytogenes, Aeromonas caviae, Morganella morganii, Aeromonas hydrophila, Neisseria gonorrhoeae, Aspergillus flavus, Neisseria meningitidis, Aspergillus nidulans, Pasteurella multocida, Aspergillus niger, Pasteurella pneumotropica, Aspergillus terreus, Propionibacterium acnes, Bacillus anthracis, Proteus mirabillis, Bacillus cereus, Providencia rettgeri, Bacillus subtilis, Pseudomonas aeruginosa, Bacteroides fragilis, Salmonella choleraesuis, Brucella melitensis, Serratia liquefaciens, Burkholderia cepacia, Serratia marcescens, Candida albicans, Staphylococcus aureus, Candida dubliniensis, Staphylococcus epidermidis, Candida glabrata, Staphylococcus haemolyticus, Candida krusei, Staphylococcus hominis, Candida parapsilosis, Staphylococcus saccharolyticus, Candida tropicalis, Staphylococcus warn-eri, Capnocytophaga canimorsus, Stenotrophomonas maltophilia, Citrobacter braakii, Streptococcus agalactiae, Citrobacter freundii, Streptococcus anginosus, Clostridium perfringens, Streptococcus bovis,Corynebacterium jeikeium, Streptococcus constellatus, Enterobacter aerogenes, Streptococcus dysgalactiae, Enterobacter cloacae, Streptococcus mutans, Enterobacter sakazakii, Streptococcus pneumoniae, Enterococcus faecalis, Streptococcus pyogenes, Enterococcus faecium, Streptococcus salivarius, Escherichia coli, Streptococcus sanguinis, Shigella sp., Streptococcus suis, Gemella haemolysans, Vibrio vulnificus, Gemella morbillorum, Yersinia enterocolitica, Haemophilus influenzae, Yersinia pestis, Kingella kingae, Yersinia pseudotuberculosis and; Klebsiella oxytoca.

As used herein the term “blood infection” may comprise a systemic blood stream infection, a sepsis, severe sepsis and/or septic shock

“Sepsis” in the context of the invention refers to a systemic response to infection. Alternatively, sepsis may be seen as the combination of SIRS with a confirmed infectious process or an infection. Sepsis may be characterized as clinical syndrome defined by the presence of both infection and a systemic inflammatory response (Levy et al. 2011). The term “sepsis” used herein includes, but is not limited to, sepsis, severe sepsis, septic shock.

The term “sepsis” used herein includes, but is not limited to, sepsis, severe sepsis, septic shock. Severe sepsis in refers to sepsis associated with organ dysfunction, hypoperfusion abnormality, or sepsis-induced hypotension. Hypoperfusion abnormalities include lactic acidosis, oliguria and acute alteration of mental status. Sepsis-induced hypotension is defined by the presence of a systolic blood pressure of less than about 90 mm Hg or its reduction by about 40 mm Hg or more from baseline in the absence of other causes for hypotension (e.g. cardiogenic shock). Septic shock is defined as severe sepsis with sepsis-induced hypotension persisting despite adequate fluid resuscitation, along with the presence of hypoperfusion abnormalities or organ dysfunction (Bone et al., CHEST 101(6): 1644-55, 1992).

The term sepsis may alternatively be defined as life-threatening organ dysfunction caused by a dysregulated host response to infection. For clinical operationalization, organ dysfunction can preferably be represented by an increase in the Sequential Organ Failure Assessment (SOFA) score of 2 points or more, which is associated with an in-hospital mortality greater than 10%. Septic shock may be defined as a subset of sepsis in which particularly profound circulatory, cellular, and metabolic abnormalities are associated with a greater risk of mortality than with sepsis alone. Patients with septic shock can be clinically identified by a vasopressor requirement to maintain a mean arterial pressure of 65 mm Hg or greater and serum lactate level greater than 2 mmol/L (>18 mg/dL) in the absence of hypovolemia.

The term “sepsis” used herein relates to all possible stages in the development of sepsis.

The term “sepsis” also includes severe sepsis or septic shock based on the SEPSIS-2 definition (Bone et al., 2009). The term “sepsis” also includes subjects falling within the SEPSIS-3 definition (Singer et al., 2016). The term “sepsis” used herein relates to all possible stages in the development of sepsis.

“Cardiovascular event” is used interchangeably herein with the term “cardiac event” and may refer to sudden cardiac death, acute coronary syndromes such as, but not limited to, plaque rupture, myocardial infarction, unstable angina, as well as non-cardiac acute arteriovascular events such as blood clots of the leg, aneurysms, stroke and other arteriovascular ischemic events where arteriovascular blood flow and oxygenation is interrupted. A cardiovascular event may also refer to an elevated heart rate, bradycardia, tachycardia or an arrhythmia such as atrial fibrillation, atrial flutter, ventricular fibrillation, and premature ventricular or atrial contractions (PVCs or PACs). Preferably a cardiovascular event is a non-fatal cardiac arrest, a congestive heart failure, a new cardia arrhythemia, an angina, a myocardial infraction, an acute myocardial infarction, myocardial injury after noncardiac surgery (MINS) or perioperative myocardial injury or a subacute ischemic heart disease

For some embodiments a cardiovascular event may refer to an event that exhibits an abnormal electrocardiogram (ECG) or an electrogram.

The term “electrocardiogram” is defined to be the heart electrical signals from one or more skin surface electrode(s) that are placed in a position to indicate the heart's electrical activity (depolarization and repolarization). An electrocardiogram segment refers to the recording of electrocardiogram data for either a specific length of time, such as 10 seconds, or a specific number of heart beats, such as 10 beats. The PQ segment of a patient's electrocardiogram is the typically a flat segment of a beat of an electrocardiogram that occurs just before the R wave.

The term “electrogram” is defined to be the heart electrical signals from one or more implanted electrode(s) that are placed in a position to indicate the heart's electrical activity (depolarization and repolarization). An electrogram segment refers to the recording of electrogram data for either a specific length of time, such as 10 seconds, or a specific number of heart beats, such as 10 beats. The PQ segment of a patient's electrogram is the typically flat segment of an electrogram that occurs just before the R wave. A beat is preferably a sub-segment of an electrogram or electrocardiogram segment containing exactly one R wave.

Heart signal parameters relate to measured or calculated values created during the processing of one or more beats of electrogram data and may be used determine whether a cardiovascular event has occurred. Heart signal parameters include PQ segment average value, ST segment average value, R wave peak value, ST deviation, ST shift, average signal strength, T wave peak height, T wave average value, T wave deviation, heart rate and R-R interval.

As used herein the term “myocardial infarction” preferably refers to an at least partial necrosis of the myocardium caused by prolonged ischemia. A type I MI is caused by an acute coronary syndrome; a rupture, ulceration, erosion or dissection of an unstable atherosclerotic plaque in one or multiple coronary vessels with subsequent intraluminal thrombosis leading to reduced myocardial blood flow. A type II MI is caused by a prolonged imbalance between myocardial oxygen supply and demand and may arise as a consequence of conditions such as cardiac arrhythmia, anaemia, respiratory failure, hypotension, hypertension or by direct toxic effects by high levels of endogenous or exogenous circulating cortisol and catecholamines. Clinically, a myocardial infarction typically defined as a new elevation of the cardiac biomarker troponin, greater than 3 times upper level of the reference range in the setting of suspected myocardial ischemia with either an ischemic symptom or an ischemic electrocardiographic (ECG) finding.

As used herein the term “myocardial injury after noncardiac surgery” or “MINS” preferably refers to a myocardial injury due to an ischemia after a non-cardiac injury. MINS preferably encompasses a wider spectrum of myocardial injuries than myocardial infarction, in particular encompassing also reversible myocardial injuries in addition to necrosis.

Preferably MINS refers to a postoperative troponin elevation judged to be due to myocardial ischemia (no evidence of nonischemic etiology) with or without ischemic symptoms. MINS preferably does not include perioperative myocardial damage originating from another etiology than ischemia, e.g. sepsis or pulmonary embolism. The postoperative thresholds for MINS depend on the type of troponin assay. For 5th generation cTnT preferably the threshold for assigning MINS may be >20 ng/L with an increase from baseline of at least 5 ng/L or >65 ng/L (Sessler et al. 2018).

A “cerebrovascular event” may preferably refer to events induced by cerebral ischemia, and especially ischemic events that are caused by insufficient supplies of oxygen to the brain, but a cerebrovascular event may also encompass hemorrhagic events that occur when blood vessels are ruptured in the brain.

The events can be focalized in a particular region of the brain, as occurs in a stroke or a transient ischemic attack (TIA), or global, as occurs in delirium. The adverse neurological event may thus be characterized by confusion or may be diagnosed as a TIA or ischemic stroke. Oxygen supplies may be compromised due to the health condition of the patient (as in certain blood disorders such as anemia), but may also be caused by the surgical event as such. A cerebrovascular event may include in particular the sum of any of the following events: occlusion or stenosis of the precerebral or cerebral arteries, a stroke or a transient ischemic attack.

A “major adverse cardiovascular or cerebrovascular event” or “MACCE” may preferably refer to a cerebrovascular or cardiovascular event selected from the group consisting of non-fatal cardiac arrest, an acute myocardial infarction, a congestive heart failure, a new (postsurgically occurring) cardiac arrhythemia, an angina or a stroke.

A “perioperative myocardial injury” or “PMI” preferably refers to any myocardial injury that occurs perioperatively. The perioperative period is the time period of a patient's surgical procedure. It commonly includes ward admission, anesthesia, surgery, and recovery. The perioperative period may also include a time relating to, occurring in, or being the period around the time of a surgical operation. Perioperative myocardial injury (PMI) seems to be a contributor to mortality after noncardiac surgery. Because the vast majority of PMIs are asymptomatic, PMI usually is missed in the absence of systematic screening (refer Puelacher et al 2018). The present invention therefore provides a useful method in determining the presence and/or risk of a PMI using the methods described herein. In some embodiments, the perioperative period is between the pre-operative and post-operative (post-surgical) period. Perioperative may in some embodiments include a part of a preoperative period, for example the time shortly before the operation, for example the 1, 2, 3, or 4 hours beginning at ward admission and preparation. In some embodiments, the invention relates to a peroperative myocardial injury. The peroperative, or intraoperative, period begins when the patient is transferred to the operating room table and ends with the transfer of a patient to the postanaesthesia care unit (PACU). Perioperative may in some embodiments include a part of a post-operative period, for example the time shortly after the operation, for example the first 1, 2, 3, or 4 hours of recovery.

Herein, a “non-fatal cardiac arrest” preferably refers to an absence of cardiac rhythm or presence of chaotic rhythm requiring any component of basic or advanced cardiac life support. An “acute myocardial infarction” preferably refers to an increase and gradual decrease in troponin level or a faster increase and decrease if creatine kinase isoenzyme as marker of myocardial necrosis in the company of at least one of the following: Ischemic symptoms, abnormal Q-waves on the ECG, ST-segment elevation or depression, coronary artery intervention (e.g. coronary angioplasty) or a an atypical decrease in an elevated troponin level detected at its peak after surgery in a patient. A “congestive heart failure” preferably refers to new in-hospital signs or symptoms after surgery of dyspnoea or fatigue, orthopnoea, paroxysmal nocturnal dyspnoea, increased jugular venous pressure, pulmonary rales on physical examination, cardiomegaly or pulmonary vascular engorgement. A “new cardiac arrhythmia” preferably refers to atrial flutter, atrial fibrillation, or second- or third-degree atrioventricular conduction block as possible evidenced by an electrocardiogram. A “angina” preferably refers to a dull diffuse substernal chest discomfort precipitated by exertion or emotion and relieved by rest or glyceryl trinitrate. A “stroke” preferably refers to an embolic, thrombotic, or hemorrhagic event lasting at least 30 in with or without persistent residual motor, sensory, or cognitive dysfunction; if the neurological symptoms continue for >24 h, a person is preferably diagnosed with stroke, and if lasting <24 h the event is defined as a transient ischemic attack (TIA).

In the context of the present invention, the term “medical treatment” or “treatment” comprises various treatments and therapeutic strategies which comprise, without limitation in regards to an infection, preferably a sepsis: anti-inflammatory strategies, antimicrobial therapies, administration of ADM-antagonists such as therapeutic antibodies, si-RNA or DNA, the extracorporal blood purification or the removal of harmful substances via apheresis, dialyses, adsorbers to prevent the cytokine storm, removal of inflammatory mediators, plasma apheresis, administration of vitamines such as vitamin C, ventilation like mechanical ventilation and non-mechanical ventilation, to provide the body with sufficient oxygen, for example, focus cleaning procedures, transfusion of blood products, infusion of colloids, renal or liver replacement, antibiotic treatment, invasive mechanical ventilation, non-invasive mechanical ventilation, renal replacement therapy, vasopressor use, fluid therapy, apheresis and measures for organ protection and in regards to cardiovascular and cerebrovascular events an anticoagulation therapy, an oxygen therapy, a lysis therapy such as a thrombolysis, a percutaneous coronary intervention, a percutaneous transluminal angioplast, a coronary artery bypass graft and/or a stent implantation. In some embodiments the medical treatment of a cardiovascular and cerebrovascular events includes the administration of nitroglycerin, acetylsalicylic acid, a beta-blocker, an ACE Inhibitor and/or dopidogrel.

In regard to perioperative myocardial infarction as means of a prevention the medical treatment may comprise in particular the administration of statins, β-blockers, α2-adrenergic agonists or other anti-ischemic agents such calcium-channel blockers or nitrates-“Renal replacement therapy” (RRT) relates to a therapy that is employed to replace the normal blood-filtering function of the kidneys. Renal replacement therapy may refer to dialysis (e.g. hemodialysis or peritoneal dialysis), hemofiltration, and hemodiafiltration. Such techniques are various ways of diverting the blood into a machine, cleaning it, and then returning it to the body. Renal replacement therapy may also refer to kidney transplantation, which is the ultimate form of replacement in that the old kidney is replaced by a donor kidney. The hemodialysis, hemofiltration, and hemodiafiltration may be continuous or intermittent and can use an arteriovenous route (in which blood leaves from an artery and returns via a vein) or a venovenous route (in which blood leaves from a vein and returns via a vein). This results in various types of RRT. For example, the renal replacement therapy may be selected from the group of, but not limited to continuous renal replacement therapy (CRRT), continuous hemodialysis (CHD), continuous arteriovenous hemodialysis (CAVHD), continuous venovenous hemodialysis (CVVHD), continuous hemofiltration (CHF), continuous arteriovenous hemofiltration (CAVH or CAVHF), continuous venovenous hemofiltration (CVVH or CVVHF), continuous hemodiafiltration (CHDF), continuous arteriovenous hemodiafiltration (CAVHDF), continuous venovenous hemodiafiltration (CVVHDF), intermittent renal replacement therapy (IRRT), intermittent hemodialysis (IHD), intermittent venovenous hemodialysis (IVVHD), intermittent hemofiltration (IHF), intermittent venovenous hemofiltration (IVVH or IVVHF), intermittent hemodiafiltration (IHDF) and intermittent venovenous hemodiafiltration (IVVHDF).

Artificial and mechanical ventilation are effective approaches to enhance proper gas exchange and ventilation and aim to save life during severe hypoxemia. Artificial ventilation relates to assisting or stimulating respiration of the subject. Artificial ventilation may be selected from the group consisting of mechanical ventilation, manual ventilation, extracorporeal membrane oxygenation (ECMO) and noninvasive ventilation (NIV). Mechanical ventilation relates to a method to mechanically assist or replace spontaneous breathing. This may involve a machine called a ventilator. Mechanical ventilation may be High-Frequency Oscillatory Ventilation or Partial Liquid Ventilation.

“Fluid management” refers to the monitoring and controlling of the fluid status of a subject and the administration of fluids to stabilize the circulation or organ vitality, by e.g. oral, enteral or intravenous fluid administration. It comprises the stabilization of the fluid and electrolyte balance or the prevention or correction of hyer- or hypovolemia as well as the supply of blood products.

Surgical emergencies/Emergency surgery are needed if a subject has a medical emergency and an immediate surgical intervention may be required to preserve survival or health status. The subject in need of emergency surgery may be selected from the group consisting of subjects suffering from acute trauma, an active uncontrolled infection, organ transplantation, organ-preventive or organ-stabilizing surgery or cancer.

Cleaning Procedures are hygienic methods to prevent subjects from infections, especially nosocomial infections, comprising desinfection of all organic and anorganic surfaces that could get in contact with a patient, such as for example, skin, objects in the patient's room, medical devices, diagnostic devices, or room air. Cleaning procedures include the use of protective clothes and units, such as mouthguards, gowns, gloves or hygiene lock, and actions like restricted patient visits. Furthermore, cleaning procedures comprise the cleaning of the patient itself and the clothes or the patient.

In the case of critical adverse events, such a severe infection or a sepsis or a severe cardiovascular or cerebrovascular event, it is very important to have an early diagnosis as well a prognosis and risk assessment of such adverse events to initiate the optimal therapy and management. The therapeutic approaches need to be individual and vary from case to case. A therapeutic monitoring is needed for a best practice therapy and is influenced by the timing of treatment, the use of combined therapies and the optimization of drug dosing or surgical means. A wrong or omitted therapy or management will increase the mortality rate hourly.

A medical treatment of the present invention may be an antibiotic treatment, wherein one or more “antibiotics” or “antibiotic agents” may be administered if an infection has been diagnosed or symptoms of an infectious disease have been determined.

Antibiotics or antibiotic agents according to the present invention also encompass potentially the antimicrobial, anti-fungal or anti-viral compounds used to treat a diagnosed infection or sepsis. The terms antimicrobial, antibiotic, and anti-infective, as may be used herein, encompass a wide variety of pharmaceutical agents that include antibacterial, antifungal, antiviral, and antiparasitic drugs. The antibiotic agents commonly applied in the treatment of any given infection, as separated into the classes of pathogen are:

Gram positive coverage: Penicillins, (ampicillin, amoxicillin), penicillinase resistant, (Dicloxacillin, Oxacillin), Cephalosporins (1st and 2nd generation), Macrolides (Erythromycin, Clarithromycin, Azithromycin), Quinolones (gatifloxacin, moxifloxacin, levofloxacin), Vancomycin, Sulfonamide/trimethoprim, Clindamycin, Tetracyclines, Chloramphenicol, Linezolid, Synercid.

Gram negative coverage: Broad spectrum penicillins (Ticarcillin, clavulanate, piperacillin, tazobactam), Cephalosporins (2nd, 3rd, and 4th generation), Aminoglycosides, Macrolides, Azithromycin, Quinolones (Ciprofloxacin), Monobactams (Azetreonam), Sulfona-mide/trimethoprim, Carbapenems (Imipenem), Chloramphenicol.

Pseudomonas coverage: Ciprofloxacin, Aminoglycosides, Some 3rd generation cephalosporins, 4th generation cephalosporins, Broad spectrum penicillins, Carbapenems.

Fungal treatments: Allyamines, Amphotericin B, Fluconazole and other Azoles, itraconazole, voriconazole, posaconazole, ravuconazole, echinocandins, Flucytosine, sordarins, chitin synthetase inhibitors, topoisomerase inhibitors, lipopeptides, pradimycins, Liposomal nystatin, Voriconazole, Echinocanidins, Imidazole, Triazole, Thiazole, Polyene.

Anti-viral treatments: Abacavir, Acyclovir (Aciclovir), activated caspase oligomerizer, Adefovir, Amantadine, Amprenavir(Agenerase), Ampligen, Arbidol, Atazanavir, Atripla, Balavir, Cidofovir, Combivir, Dolutegravir, Darunavir, Delavirdine, Didanosine, Double-stranded RNA, Docosanol, Edoxudine, Efavirenz, Emtricitabine, Enfuvirtide, Entecavir, Ecoliever, Famciclovir, Fixed dose combination (antiretroviral), Fomivirsen, Fosamprenavir, Foscarnet, Fosfonet, Fusion inhibitor, Ganciclovir, Ibacitabine, Imunovir, Idoxuridine, Imiquimod, Indinavir, Inosine, Integrase inhibitor, Interferon type III, Interferon type II, Interferon type I, Interferon, Lamivudine, Lopinavir, Loviride, Maraviroc, Moroxydine, Methisazone, Morpholinos, Nelfinavir, Nevirapine, Nexavir, Nitazoxanide, Nucleoside analogues, Novir, Oseltamivir (Tamiflu), Peginterferon alfa-2a, Penciclovir, Peramivir, Pleconaril, Podophyllotoxin, Protease inhibitor (pharmacology), Raltegravir, Reverse transcriptase inhibitor, Ribavirin, Ribozymes, Rifampicin, Rimantadine, Ritonavir, RNase H, protease inhibitors, Pyramidine, Saquinavir, Sofosbuvir, Stavudine, Synergistic enhancer (antiretroviral), Telaprevir, Tenofovir, Tenofovir disoproxil, Tipranavir, Trifluridine, Trizivir, Tromantadine, Truvada, Valaciclovir (Valtrex), Valganciclovir, Vicriviroc, Vidarabine, Viramidine, Zalcitabine, Zanamivir (Relenza), Zidovudine.

Furthermore, antibiotic agents comprise bacteriophages for treatment of bacterial infections, synthetic antimicrobial peptides or iron-antagonists/iron chelator. Also, therapeutic antibodies or antagonist against pathogenic structures like anti-VAP-antibodies, anti-resistant clone vaccination, administration of immune cells, such as in vitro primed or modulated T-effector cells, are antibiotic agents that represent treatment options for critically ill patients, such as sepsis patients. Further antibiotic agents/treatments or therapeutic strategies against infection or for the prevention of new infections include the use of antiseptics, decontamination products, anti-virulence agents like liposomes, sanitation, wound care, surgery.

It is also possible to combine several of the aforementioned antibiotic agents or treatments strategies.

According to the present invention proADM, PCT and/or proET-1 and/or optionally other markers or clinical scores are employed as markers for prognosis, diagnosis, risk assessment and risk stratification of an adverse event in the health of a postsurgical patient as well as optionally additional therapy guidance, therapy stratification and/or therapy control.

A skilled person is capable of obtaining or developing means for the identification, measurement, determination and/or quantification of any one of the above proADM, PCT, and/or pro ET-1 molecules, or fragments or variants thereof, as well as the other markers of the present invention according to standard molecular biological practice.

The level of proADM, PCT, and/or pro ET-1 or fragments thereof as well as the levels of other markers of the present invention can be determined by any assay that reliably determines the concentration of the marker. Particularly, mass spectrometry (MS) and/or immunoassays can be employed as exemplified in the appended examples. As used herein, an immunoassay is a biochemical test that measures the presence or concentration of a macromolecule/polypeptide in a solution through the use of an antibody or antibody binding fragment or immunoglobulin.

Methods of determining proADM, PCT, and/or proET-1 or other the markers used in the context of the present invention are intended in the present invention. By way of example, a method may be employed selected from the group consisting of mass spectrometry (MS), luminescence immunoassay (LIA), radioimmunoassay (RIA), chemiluminescence- and fluorescence-immunoassays, enzyme immunoassay (EIA), Enzyme-linked immunoassays (ELISA), luminescence-based bead arrays, magnetic beads based arrays, protein microarray assays, rapid test formats such as for instance immunochromatographic strip tests, rare cryptate assay, and automated systems/analyzers.

Determination of proADM, PCT, and/or proET-1 and optionally other markers based on antibody recognition is a preferred embodiment of the invention. As used herein, the term, “antibody” refers to immunoglobulin molecules and immunologically active portions of immunoglobulin (Ig) molecules, i.e., molecules that contain an antigen binding site that specifically binds (immuno reacts with) an antigen. According to the invention, the antibodies may be monoclonal as well as polyclonal antibodies. Particularly, antibodies that are specifically binding to at least proADM, PCT, and/or proET-1 or fragments thereof are used.

An antibody is considered to be specific, if its affinity towards the molecule of interest, e.g. proADM, PCT, and/or proET-1, or the fragment thereof is at least 50-fold higher, preferably 100-fold higher, most preferably at least 1000-fold higher than towards other molecules comprised in a sample containing the molecule of interest. It is well known in the art how to develop and to select antibodies with a given specificity. In the context of the invention, monoclonal antibodies are preferred. The antibody or the antibody binding fragment binds specifically to the herein defined markers or fragments thereof. In particular, the antibody or the antibody binding fragment binds to the herein defined peptides of proADM, PCT, and/or proET-1. Thus, the herein defined peptides can also be epitopes to which the antibodies specifically bind. Further, an antibody or an antibody binding fragment is used in the methods and kits of the invention that binds specifically to ADM or proADM, particularly to MR-proADM, to PCT and/or to proET-1, particularly to CT-proET1.

Further, an antibody or an antibody binding fragment is used in the methods and kits of the invention that binds specifically to proADM, PCT, and/or proET-1 or fragments thereof and optionally to other markers of the present inventions. Exemplary immunoassays can be luminescence immunoassay (LIA), radioimmunoassay (RIA), chemiluminescence- and fluorescence-immunoassays, enzyme immunoassay (EIA), Enzyme-linked immunoassays (ELISA), luminescence-based bead arrays, magnetic beads based arrays, protein microarray assays, rapid test formats, rare cryptate assay. Further, assays suitable for point-of-care testing and rapid test formats such as for instance immune-chromatographic strip tests can be employed. Automated immunoassays are also intended, such as the KRYPTOR assay.

Alternatively, instead of antibodies, other capture molecules or molecular scaffolds that specifically and/or selectively recognize proADM, PCT, and/or pro ET-1 may be encompassed by the scope of the present invention. Herein, the term “capture molecules” or “molecular scaffolds” comprises molecules which may be used to bind target molecules or molecules of interest, i.e. analytes (e.g. proADM, proADM, MR-proADM, PCT, proET-1 or CT-proET-1), from a sample.

Capture molecules must thus be shaped adequately, both spatially and in terms of surface features, such as surface charge, hydrophobicity, hydrophilicity, presence or absence of lewis donors and/or acceptors, to specifically bind the target molecules or molecules of interest. Hereby, the binding may, for instance, be mediated by ionic, van-der-Waals, pi-pi, sigma-pi, hydrophobic or hydrogen bond interactions or a combination of two or more of the aforementioned interactions or covalent interactions between the capture molecules or molecular scaffold and the target molecules or molecules of interest. In the context of the present invention, capture molecules or molecular scaffolds may for instance be selected from the group consisting of a nucleic acid molecule, a carbohydrate molecule, a PNA molecule, a protein, a peptide and a glycoprotein. Capture molecules or molecular scaffolds include, for example, aptamers, DARpins (Designed Ankyrin Repeat Proteins). Affimers and the like are included.

In certain aspects of the invention, the method comprises an immunoassay comprising the steps of:

a) contacting the sample with
i. a first antibody or an antigen-binding fragment or derivative thereof specific for a first epitope of proADM, PCT and/or proET-1, and
ii. a second antibody or an antigen-binding fragment or derivative thereof specific for a second epitope of proADM, PCT and/or proET-1; and

b) detecting the binding of the two antibodies or antigen-binding fragments or derivates thereof to proADM, PCT and/or proET-1.

Preferably, one of the antibodies can be labeled and the other antibody can be bound to a solid phase or can be bound selectively to a solid phase. In a particularly preferred aspect of the assay, one of the antibodies is labeled while the other is either bound to a solid phase or can be bound selectively to a solid phase. The first antibody and the second antibody can be present dispersed in a liquid reaction mixture, and wherein a first labeling component which is part of a labeling system based on fluorescence or chemiluminescence extinction or amplification is bound to the first antibody, and a second labeling component of said labeling system is bound to the second antibody so that, after binding of both antibodies to proADM, PCT and/or ET-1 or fragments thereof to be detected, a measurable signal which permits detection of the resulting sandwich complexes in the measuring solution is generated. The labeling system can comprise a rare earth cryptate or chelate in combination with a fluorescent or chemiluminescent dye, in particular of the cyanine type.

In a preferred embodiment, the method is executed as heterogeneous sandwich immunoassay, wherein one of the antibodies is immobilized on an arbitrarily chosen solid phase, for example, the walls of coated test tubes (e.g. polystyrol test tubes; coated tubes; CT) or microtiter plates, for example composed of polystyrol, or to particles, such as for instance magnetic particles, whereby the other antibody has a group resembling a detectable label or enabling for selective attachment to a label, and which serves the detection of the formed sandwich structures. A temporarily delayed or subsequent immobilization using suitable solid phases is also possible.

The method according to the present invention can furthermore be embodied as a homogeneous method, wherein the sandwich complexes formed by the antibody/antibodies and the marker, proADM, PCT and/or ET-1 or a fragment thereof, which is to be detected remains suspended in the liquid phase. In this case it is preferred, that when two antibodies are used, both antibodies are labeled with parts of a detection system, which leads to generation of a signal or triggering of a signal if both antibodies are integrated into a single sandwich. Such techniques are to be embodied in particular as fluorescence enhancing or fluorescence quenching detection methods. A particularly preferred aspect relates to the use of detection reagents which are to be used pair-wise, such as for example the ones which are described in U.S. Pat. No. 4,882,733, EP0180492 or EP0539477 and the prior art cited therein. In this way, measurements in which only reaction products comprising both labeling components in a single immune-complex directly in the reaction mixture are detected, become possible. For example, such technologies are offered under the brand names TRACE® (Time Resolved Amplified Cryptate Emission) or KRYPTOR®, implementing the teachings of the above-cited applications. Therefore, in particular preferred aspects, a diagnostic device is used to carry out the herein provided method. For example, the level of proADM, PCT and/or ET-1 or fragments thereof and/or the level of any further marker of the herein provided method, is determined. In particular preferred aspects, the diagnostic device is KRYPTOR® or correlating automated systems.

The level of the marker of the present invention, e.g. proADM, PCT and/or ET-1 or fragments thereof, or other markers, can also be determined by a mass spectrometric (MS) based methods. Such a method may comprise detecting the presence, amount or concentration of one or more modified or unmodified fragment peptides of e.g. proADM, PCT or proET-1 in said biological sample or a protein digest (e.g. tryptic digest) from said sample, and optionally separating the sample with chromatographic methods, and subjecting the prepared and optionally separated sample to MS analysis. For example, selected reaction monitoring (SRM), multiple reaction monitoring (MRM) or parallel reaction monitoring (PRM) mass spectrometry may be used in the MS analysis, particularly to determine the amounts of proADM, PCT or proET-1 or fragments thereof.

Herein, the term “mass spectrometry” or “MS” refers to an analytical technique to identify compounds by their mass. In order to enhance the mass resolving and mass determining capabilities of mass spectrometry, the samples can be processed prior to MS analysis. Accordingly, the invention relates to MS detection methods that can be combined with immuno-enrichment technologies, methods related to sample preparation and/or chromatographic methods, preferably with liquid chromatography (LC), more preferably with high performance liquid chromatography (HPLC) or ultra high performance liquid chromatography (UHPLC). Sample preparation methods comprise techniques for lysis, fractionation, digestion of the sample into peptides, depletion, enrichment, dialysis, desalting, alkylation and/or peptide reduction. However, these steps are optional. The selective detection of analyte ions may be conducted with tandem mass spectrometry (MS/MS). Tandem mass spectrometry is characterized by mass selection step (as used herein, the term “mass selection” denotes isolation of ions having a specified m/z or narrow range of m/z's), followed by fragmentation of the selected ions and mass analysis of the resultant product (fragment) ions.

The skilled person is aware how quantify the level of a marker in the sample by mass spectrometric methods. For example, relative quantification “rSRM” or absolute quantification can be employed as described above.

Moreover, the levels (including reference levels) can be determined by mass spectrometric based methods, such as methods determining the relative quantification or determining the absolute quantification of the protein or fragment thereof of interest.

Relative quantification “rSRM” may be achieved by:

1. Determining increased or decreased presence of the target protein by comparing the SRM (Selected reaction monitoring) signature peak area from a given target fragment peptide detected in the sample to the same SRM signature peak area of the target fragment peptide in at least a second, third, fourth or more biological samples.

2. Determining increased or decreased presence of target protein by comparing the SRM signature peak area from a given target peptide detected in the sample to SRM signature peak areas developed from fragment peptides from other proteins, in other samples derived from different and separate biological sources, where the SRM signature peak area comparison between the two samples for a peptide fragment are normalized for e.g to amount of protein analyzed in each sample.

3. Determining increased or decreased presence of the target protein by comparing the SRM signature peak area for a given target peptide to the SRM signature peak areas from other fragment peptides derived from different proteins within the same biological sample in order to normalize changing levels of histones protein to levels of other proteins that do not change their levels of expression under various cellular conditions.

4. These assays can be applied to both unmodified fragment peptides and to modified fragment peptides of the target proteins, where the modifications include, but are not limited to phosphorylation and/or glycosylation, acetylation, methylation (mono, di, tri), citrullination, ubiquitinylation and where the relative levels of modified peptides are determined in the same manner as determining relative amounts of unmodified peptides.

Absolute quantification of a given peptide may be achieved by:

1. Comparing the SRM/MRM signature peak area for a given fragment peptide from the target proteins in an individual biological sample to the SRM/MRM signature peak area of an internal fragment peptide standard spiked into the protein lysate from the biological sample. The internal standard may be a labeled synthetic version of the fragment peptide from the target protein that is being interrogated or the labeled recombinant protein. This standard is spiked into a sample in known amounts before (mandatory for the recombinant protein) or after digestion, and the SRM/MRM signature peak area can be determined for both the internal fragment peptide standard and the native fragment peptide in the biological sample separately, followed by comparison of both peak areas. This can be applied to unmodified fragment peptides and modified fragment peptides, where the modifications include but are not limited to phosphorylation and/or glycosylation, acetylation, methylation (e.g. mono-, di-, or tri-methylation), citrullination, ubiquitinylation, and where the absolute levels of modified peptides can be determined in the same manner as determining absolute levels of unmodified peptides.

2. Peptides can also be quantified using external calibration curves. The normal curve approach uses a constant amount of a heavy peptide as an internal standard and a varying amount of light synthetic peptide spiked into the sample. A representative matrix similar to that of the test samples needs to be used to construct standard curves to account for a matrix effect. Besides, reverse curve method circumvents the issue of endogenous analyte in the matrix, where a constant amount of light peptide is spiked on top of the endogenous analyte to create an internal standard and varying amounts of heavy peptide are spiked to create a set of concentration standards. Test samples to be compared with either the normal or reverse curves are spiked with the same amount of standard peptide as the internal standard spiked into the matrix used to create the calibration curve.

The invention further relates to kits, the use of the kits and methods wherein such kits are used. The invention relates to kits for carrying out the herein above and below provided methods. The herein provided definitions, e.g. provided in relation to the methods, also apply to the kits of the invention. In particular, the invention relates to kits for prognosis, diagnosis risk assessment or risk stratification of an adverse event in the health of a postsurgical patient, wherein said kit comprises
detection reagents for determining the level of at least one biomarker selected from the group consisting of proADM, PCT and proET-1 or fragment(s) thereof, and
reference data for the likelihood of a postsurgical adverse event, in particular reference data for threshold or cut-off value(s), wherein said reference data is preferably stored on a computer readable medium and/or employed in the form of computer executable code configured for comparing the determined levels of the least one biomarker selected from the group consisting of proADM, PCT and proET-1 or fragment(s) thereof with the threshold or cut-off value(s).

As used herein, “reference data” comprise reference level(s) of proADM, PCT and/or pro ET-1 and optionally further markers as described herein. The levels of proADM, PCT and/or proET-1 and in the sample of the subject can be compared to the reference levels comprised in the reference data of the kit. The reference levels are herein described above and are exemplified also in the appended examples. The reference data can also include a reference sample to which the level of proADM and optionally PCT, lactate and/or C-reactive protein is compared. The reference data can also include an instruction manual how to use the kits of the invention.

The kit may additionally comprise items useful for obtaining a sample, such as a blood sample, for example the kit may comprise a container, wherein said container comprises a device for attachment of said container to a canula or syringe, is a syringe suitable for blood isolation, exhibits an internal pressure less than atmospheric pressure, such as is suitable for drawing a pre-determined volume of sample into said container, and/or comprises additionally detergents, chaotropic salts, ribonuclease inhibitors, chelating agents, such as guanidinium isothiocyanate, guanidinium hydrochloride, sodium dodecylsulfate, polyoxyethylene sorbitan monolaurate, RNAse inhibitor proteins, and mixtures thereof, and/or A filter system containing nitro-cellulose, silica matrix, ferromagnetic spheres, a cup retrieve spill over, trehalose, fructose, lactose, mannose, poly-ethylen-glycol, glycerol, EDTA, TRIS, limonene, xylene, benzoyl, phenol, mineral oil, anilin, pyrol, citrate, and mixtures thereof.

As used herein, the “detection reagent” or the like are reagents that are suitable to determine the herein described marker(s), e.g. of proADM, PCT and/or proET-1. Such exemplary detection reagents are, for example, ligands, e.g. antibodies or fragments thereof, which specifically bind to the peptide or epitopes of the herein described marker(s). Such ligands might be used in immunoassays as described above. Further reagents that are employed in the immunoassays to determine the level of the marker(s) may also be comprised in the kit and are herein considered as detection reagents. Detection reagents can also relate to reagents that are employed to detect the markers or fragments thereof by MS based methods. Such detection reagent can thus also be reagents, e.g. enzymes, chemicals, buffers, etc, that are used to prepare the sample for the MS analysis. A mass spectrometer can also be considered as a detection reagent. Detection reagents according to the invention can also be calibration solution(s), e.g. which can be employed to determine and compare the level of the marker(s).

The sensitivity and specificity of a diagnostic and/or prognostic test depends on more than just the analytical “quality” of the test, they also depend on the definition of what constitutes an abnormal result. In practice, Receiver Operating Characteristic curves (ROC curves), are typically calculated by plotting the value of a variable versus its relative frequency in “normal” (i.e. apparently healthy individuals not having an infection and “disease” populations, e.g. subjects having an infection. For any particular marker (like proADM, PCT and/or proET-1), a distribution of marker levels for subjects with and without a disease/condition will likely overlap. Under such conditions, a test does not absolutely distinguish normal from disease with 100% accuracy, and the area of overlap might indicate where the test cannot distinguish normal from disease. A threshold is selected, below which the test is considered to be abnormal and above which the test is considered to be normal or below or above which the test indicates a specific condition, e.g. infection or a cardiovascular or cerebrovascular event. The area under the ROC curve is a measure of the probability that the perceived measurement will allow correct identification of a condition. ROC curves can be used even when test results do not necessarily give an accurate number. As long as one can rank results, one can create a ROC curve. For example, results of a test on “disease” samples might be ranked according to degree (e.g. 1=low, 2=normal, and 3=high). This ranking can be correlated to results in the “normal” population, and a ROC curve created. These methods are well known in the art; see, e.g., Hanley et al. 1982. Radiology 143: 29-36. Preferably, a threshold is selected to provide a ROC curve area of greater than about 0.5, more preferably greater than about 0.7, still more preferably greater than about 0.8, even more preferably greater than about 0.85, and most preferably greater than about 0.9. The term “about” in this context refers to +/−5% of a given measurement.

The horizontal axis of the ROC curve represents (1-specificity), which increases with the rate of false positives. The vertical axis of the curve represents sensitivity, which increases with the rate of true positives. Thus, fora particular cut-off selected, the value of (1-specificity) may be determined, and a corresponding sensitivity may be obtained. The area under the ROC curve is a measure of the probability that the measured marker level will allow correct identification of a disease or condition. Thus, the area under the ROC curve can be used to determine the effectiveness of the test.

The methods of the present invention may in part be computer-implemented. For example, the step of comparing the detected level of a marker, e.g. the proADM, PCT and/or pro ET-1 or fragments thereof, with a reference level can be performed in a computer system. In the computer-system, the determined level of the marker(s) can be combined with other marker levels and/or parameters of the subject in order to calculate a score, which is indicative for the diagnosis, prognosis, risk assessment and/or risk stratification. For example, the determined values may be entered (either manually by a health professional or automatically from the device(s) in which the respective marker level(s) has/have been determined) into the computer-system. The computer-system can be directly at the point-of-care (e.g. primary care, ICU or ED) or it can be at a remote location connected via a computer network (e.g. via the internet, or specialized medical cloud-systems, optionally combinable with other IT-systems or platforms such as hospital information systems (HIS)). Typically, the computer-system will store the values (e.g. marker level or parameters such as age, blood pressure, weight, sex, etc. or clinical scoring systems such as SOFA, qSOFA, BMI etc.) on a computer-readable medium and calculate the score based-on pre-defined and/or pre-stored reference levels or reference values. The resulting score will be displayed and/or printed for the user (typically a health professional such as a physician). Alternatively or in addition, the associated prognosis, diagnosis, assessment, treatment guidance, patient management guidance or stratification will be displayed and/or printed for the user (typically a health professional such as a physician).

In one embodiment of the invention, a software system can be employed, in which a machine learning algorithm is evident, preferably to identify hospitalized patients at risk for sepsis, severe sepsis and septic shock using data from electronic health records (EHRs). A machine learning approach can be trained on a random forest classifier using EHR data (such as labs, biomarker expression, vitals, and demographics) from patients. Machine learning is a type of artificial intelligence that provides computers with the ability to learn complex patterns in data without being explicitly programmed, unlike simpler rule-based systems. Earlier studies have used electronic health record data to trigger alerts to detect clinical deterioration in general. In one embodiment of the invention the processing of proADM, PCT and/or proET-1 levels may be incorporated into appropriate software for comparison to existing data sets, for example proADM, PCT and/or proET-1 levels may also be processed in machine learning software to assist in diagnosing or prognosing the occurrence of an adverse event.

As used herein, the terms “comprising” and “including” or grammatical variants thereof are to be taken as specifying the stated features, integers, steps or components but do not preclude the addition of one or more additional features, integers, steps, components or groups thereof. This term encompasses the terms “consisting of” and “consisting essentially of”.

Thus, the terms “comprising”/“including”/“having” mean that any further component (or likewise features, integers, steps and the like) can/may be present. The term “consisting of” means that no further component (or likewise features, integers, steps and the like) is present.

The term “consisting essentially of” or grammatical variants thereof when used herein are to be taken as specifying the stated features, integers, steps or components but do not preclude the addition of one or more additional features, integers, steps, components or groups thereof but only if the additional features, integers, steps, components or groups thereof do not materially alter the basic and novel characteristics of the claimed composition, device or method.

Thus, the term “consisting essentially of” means those specific further components (or likewise features, integers, steps and the like) can be present, namely those not materially affecting the essential characteristics of the composition, device or method. In other words, the term “consisting essentially of” (which can be interchangeably used herein with the term “comprising substantially”), allows the presence of other components in the composition, device or method in addition to the mandatory components (or likewise features, integers, steps and the like), provided that the essential characteristics of the device or method are not materially affected by the presence of other components.

The term “method” refers to manners, means, techniques and procedures for accomplishing a given task including, but not limited to, those manners, means, techniques and procedures either known to, or readily developed from known manners, means, techniques and procedures by practitioners of the chemical, biological and biophysical arts.

### FIGURES

The present invention is further described by reference to the following figures. The figures exemplify non-limiting and potentially preferred embodiments, presented for further illustration of the invention.

### BRIEF DESCRIPTION OF THE FIGURES

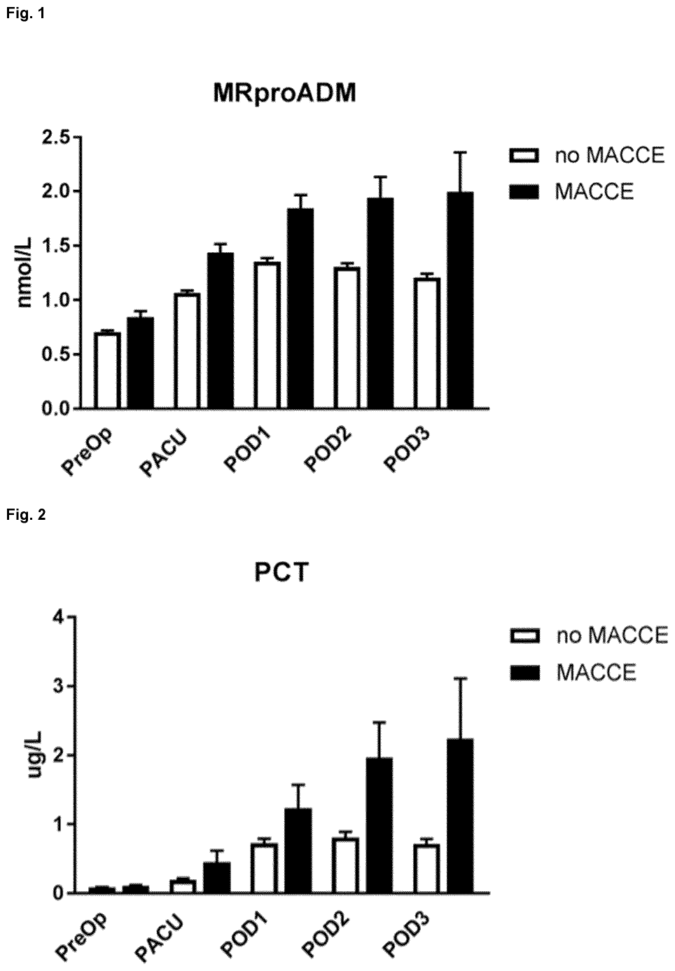

FIG. 1 Plasma levels of MR-proADM, at all sampling points in patients with and without MACCE, presented as mean with standard error of the mean (SEM).

FIG. 2 Plasma levels of PCT, at all sampling points in patients with and without MACCE, presented as mean with standard error of the mean (SEM).

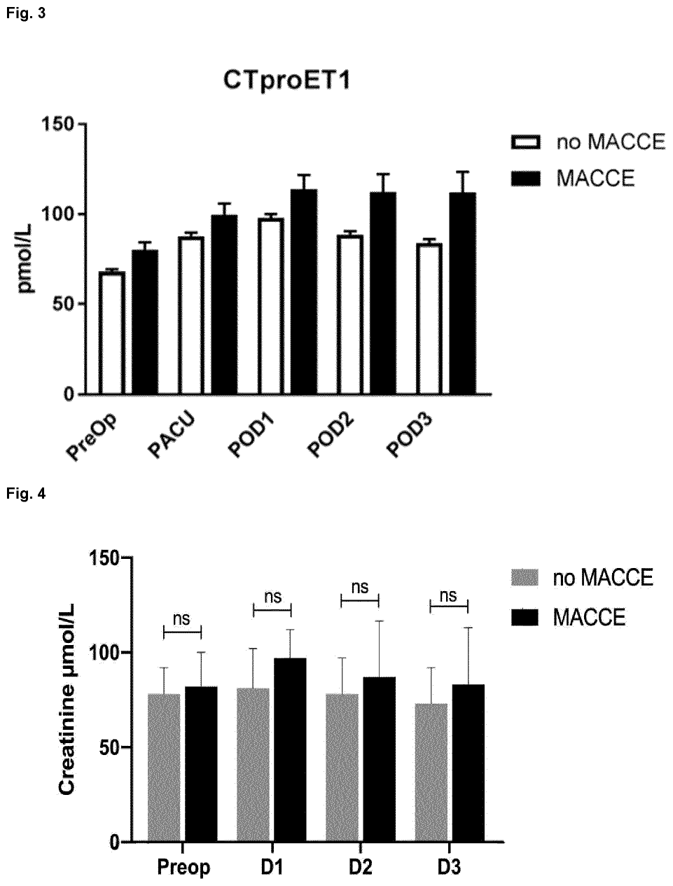

FIG. 3 Plasma levels of CT-proET-1, at all sampling points in patients with and without MACCE, presented as mean with standard error of the mean (SEM).

FIG. 4 Plasma levels of creatinine, at all sampling points in patients with and without MACCE, presented as median with interquartile range (IQR).

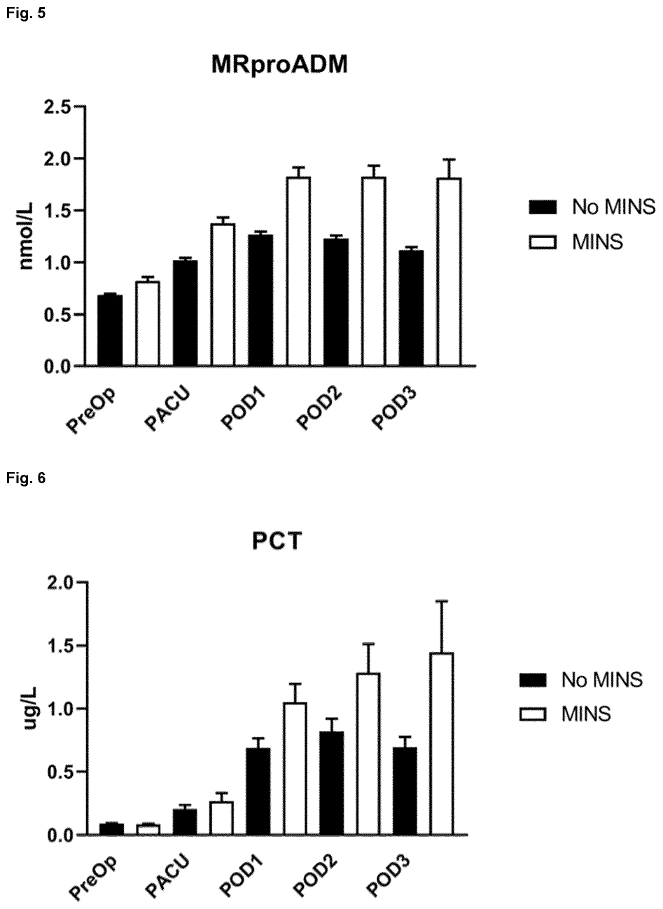

FIG. 5 Plasma levels of MR-proADM, at all sampling points in patients with and without MINS, presented as mean with standard error of the mean (SEM).

FIG. 6 Plasma levels of PCT, at all sampling points in patients with and without MINS, presented as mean with standard error of the mean (SEM).

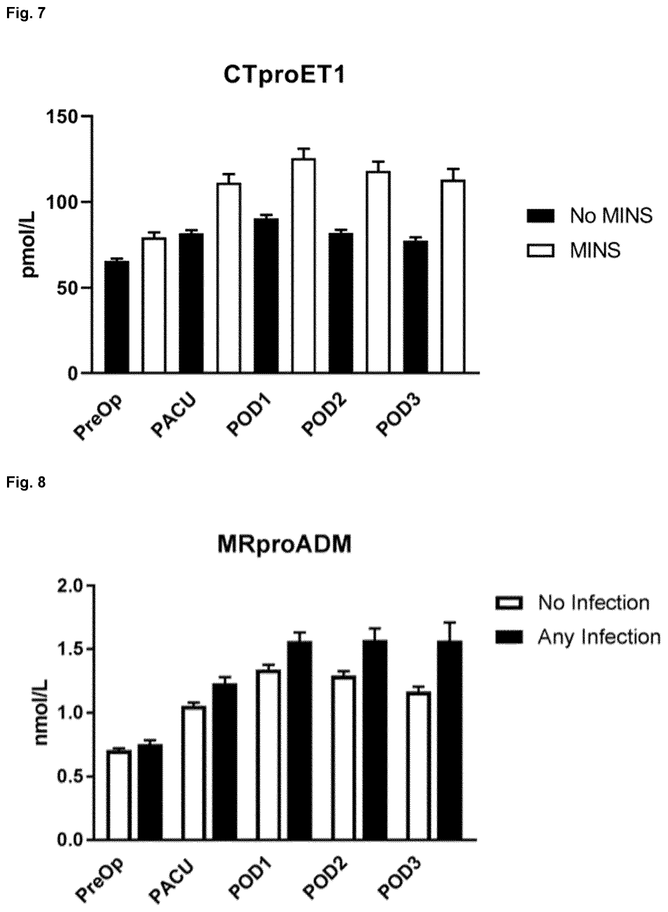

FIG. 7 Plasma levels of CT-proET-1, at all sampling points in patients with and without MINS, presented as mean with standard error of the mean (SEM).

FIG. 8 Plasma levels of MR-proADM, at all sampling points in patients with and without any infection, presented as mean with standard error of the mean (SEM).

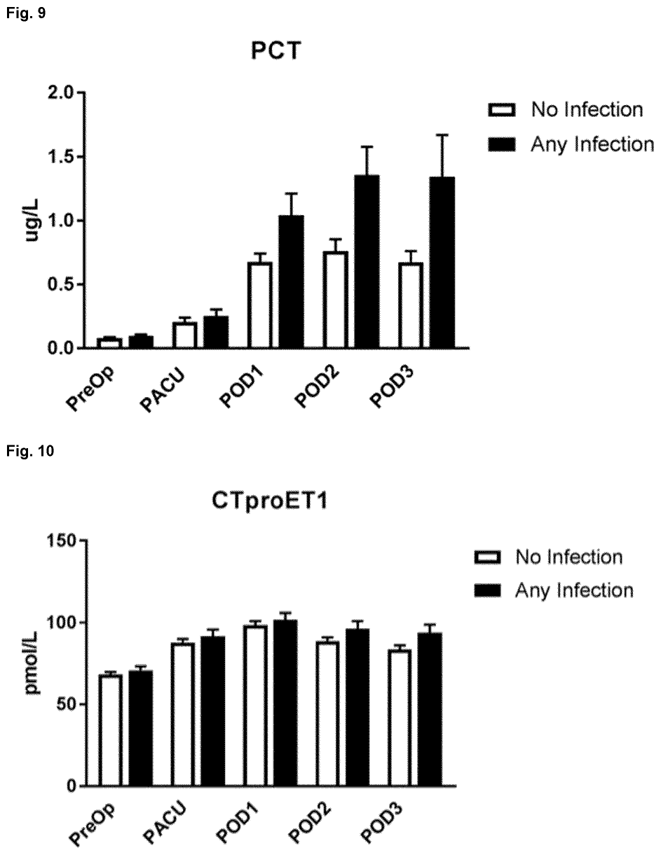

FIG. 9 Plasma levels of PCT, at all sampling points in patients with and without any infection, presented as mean with standard error of the mean (SEM).

FIG. 10 Plasma levels of CT-proET-1, at all sampling points in patients with and without any infection, presented as mean with standard error of the mean (SEM).

FIG. 11 Plasma levels of MR-proADM, at all sampling points in patients with and without a blood infection, presented as mean with standard error of the mean (SEM).

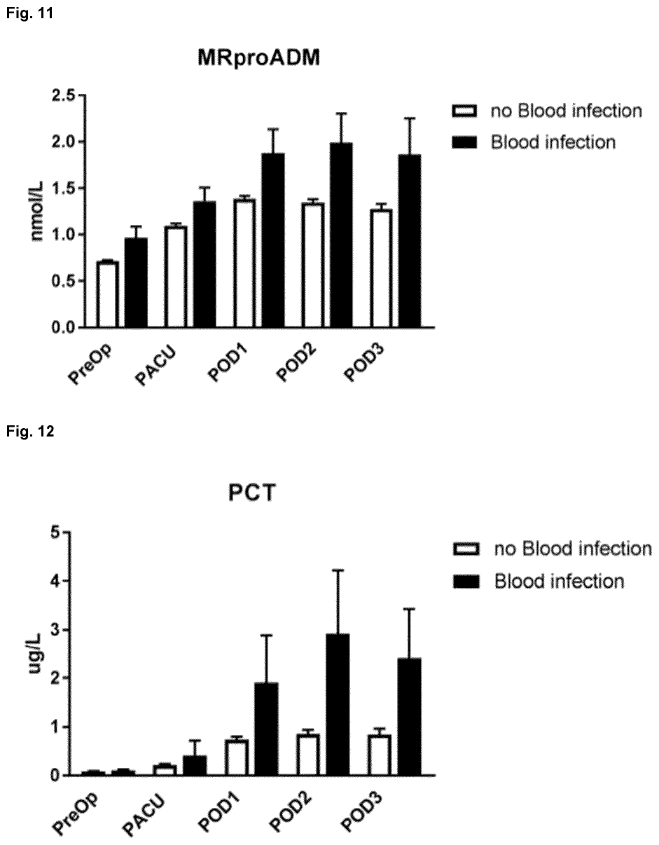

FIG. 12 Plasma levels of PCT, at all sampling points in patients with and without a blood infection, presented as mean with standard error of the mean (SEM).

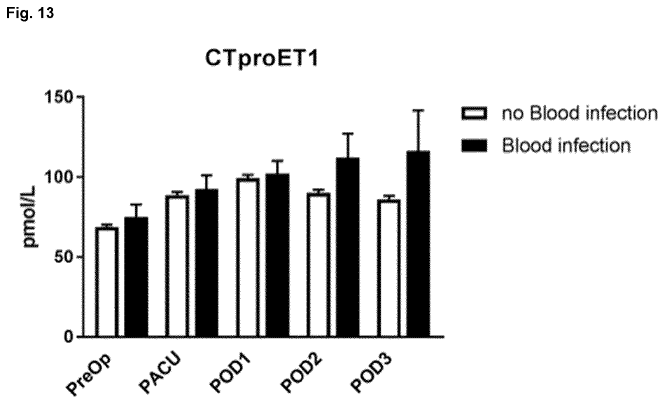

FIG. 13 Plasma levels of CT-proET-1, at all sampling points in patients with and without a blood infection, presented as mean with standard error of the mean (SEM).

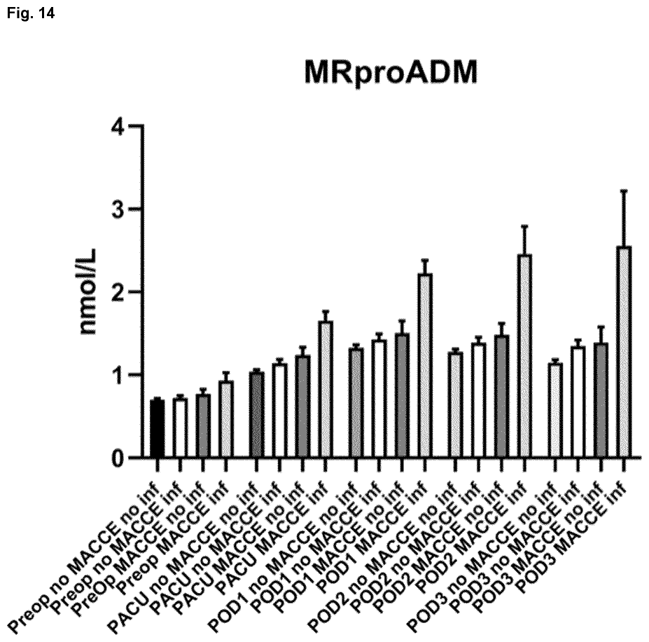

FIG. 14 Plasma levels of MR-proADM, at all sampling points in patients with an infection and a MACCE compared to patients that suffered of only one or neither one of the adverse events, presented as mean with standard error of the mean (SEM).

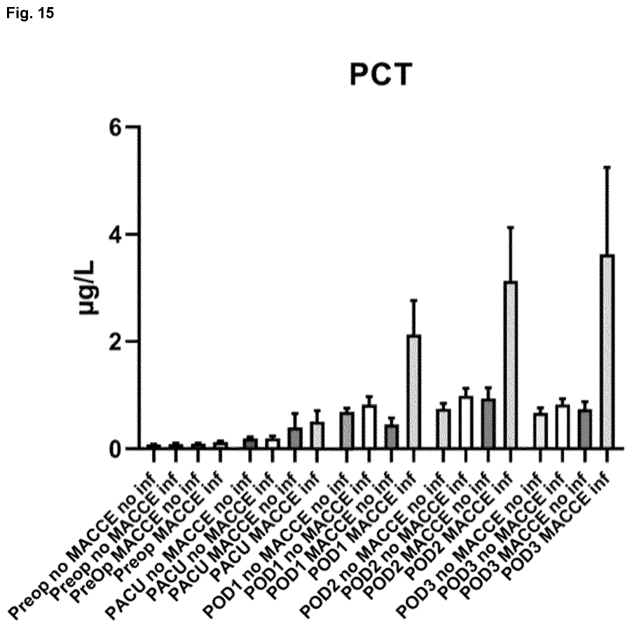

FIG. 15 Plasma levels of PCT, at all sampling points in patients with an infection and a MACCE compared to patients that suffered of only one or neither one of the adverse events, presented as mean with standard error of the mean (SEM).

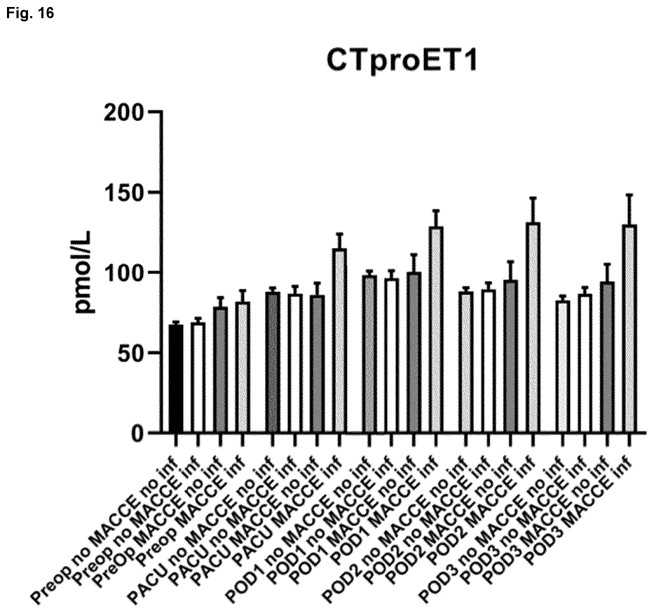

FIG. 16 Plasma levels of CT-proET-1, at all sampling points in patients with an infection and a MACCE compared to patients that suffered of only one or neither one of the adverse events, presented as mean with standard error of the mean (SEM).

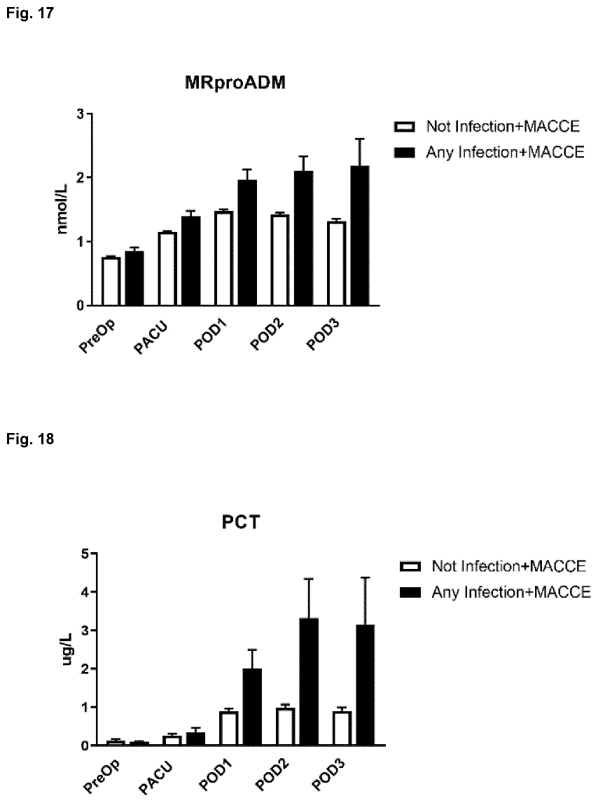

FIG. 17 Plasma levels of MR-proADM, at all sampling points in patients with MACCE and no infection compared to patients with MACCE and any infection, presented as mean with standard error of the mean (SEM).

FIG. 18 Plasma levels of PCT, at all sampling points in patients with MACCE and no infection compared to patients with MACCE and any infection, presented as mean with standard error of the mean (SEM).

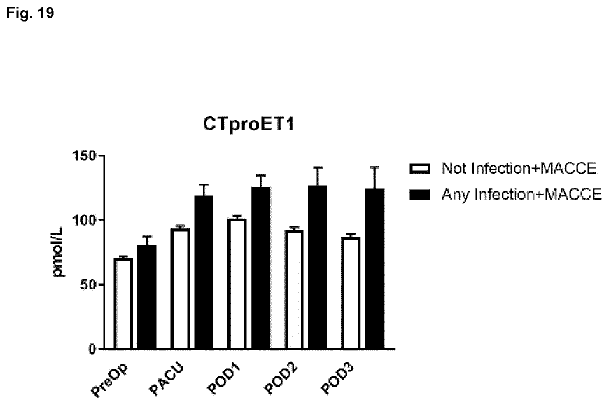

FIG. 19 Plasma levels of CT-proET-1, at all sampling points in patients with MACCE and no infection compared to patients with MACCE and any infection, presented as mean with standard error of the mean (SEM).

### EXAMPLES

The present invention is further described by reference to the following non-limiting examples. The examples describe non-limiting and practical embodiments, presented for further illustration of the invention.

### METHODS OF THE EXAMPLES

### Study Design and Patients:

This study is a part of the MINSS-study, Myocardial Injury in Noncardiac Surgery in Sweden, aiming to find a relation between perioperative variables and the occurrence of adverse outcomes in Swedish patients undergoing elective, major abdominal surgery. The MINSS-study is multicenter, prospective cohort study in several Swedish hospitals, for which the subjects were consecutively between April 2017 and October 2018. Patients aged 50 or older, undergoing elective abdominal surgery rated as major or major/complex by SORT, Surgical Outcome Risk Tool (Protopapa et al. 2014) were eligible. The procedure also had to be performed under general anesthesia and require at least one overnight stay.

Eligible patients were identified by screening daily patients list in preoperative assessment clinics. Patients were informed of the study objectives, methods, expected benefits, potential dangers and the possibility of withdrawal at any time before consenting to participate.

Informed written consent was obtained from all patients before being included.

Participants could be included only once and patients were excluded if they were not able to give an informed consent or underwent one of the following types of abdominal surgery; transplantation, trauma, endocrine, vascular or endovascular.

Included patients were excluded if the elective surgery was cancelled because the patient died, or if another non-elective surgery was performed prior to the elective one.

### Study Endpoints

MACCE within 30 days, and MINS within 3 days of surgery were predefined study endpoints. MINS is defined as a peak 5th generation cTnT>65 ng/L or >20 ng/L with an elevation >5 ng/L from preoperative levels. MACCE was defined as one or more of the endpoints listed in Table 9 (Devereaux et al. 2005).

### Date Collection and Analysis

A 12-lead ECG was obtained at 5 sample points; preoperatively (<24 h prior to anaesthesia), postoperatively (within 24 hours of surgery) and on days 1, 2 and 3 after surgery. At the same time, an arterial or venous blood sample was obtained using an 8.5 ml EDTA tube. Blood samples were sent to the local clinical chemistry laboratory where they were centrifuged, plasma was frozen in aliquots at −80° C. and stored until batch-analyses of plasma concentration of CT-proET-1, MR-proADM, PCT and cTnT were performed.

New ECG changes, according to table 2 was considered indicative of myocardial ischemia. All ECGs were analysed by investigators blinded to the results of hsTnT and CT-proET-1.

The occurrence of MACCE was reviewed on day 1, 2 and 3 through medical charts and at 30 days after surgery via a telephone interview and/or by consulting the patient's medical records.

The incidence of MINS was calculated from analysed cTnT plasma levels. The mortality rate was assessed using the Swedish population register.

The occurrence of infection or sepsis was reviewed on day 1, 2 and 3 through trained adjudicators and microbial identification and at 30 days after surgery via a telephone interview and/or by consulting the patient's medical records.

In addition, the general health status of the patients was assessed using the American Society of Anesthesiologists (ASA) Physical Status Classification System:

| | | |
|---|---|---|
| ASA I | A normal healthy patient | Healthy, non-smoking, no or minimal alcohol use |
| ASA II | A patient with mild | Mild diseases only without substantive functional |
| | systemic disease | limitations. Examples include (but not limited to): |
| | | current smoker, social alcohol drinker, pregnancy, |
| | | obesity (30 < BMI < 40), well-controlled DM/HTN, |
| | | mild lung disease |
| ASA III | A patient with severe | Substantive functional limitations; One or more |
| | systemic disease | moderate to severe diseases. Examples include (but |
| | | not limited to): poorly controlled DM or HTN, COPD, |
| | | morbid obesity (BMI ≥ 40), active hepatitis, alcohol |
| | | dependence or abuse, implanted pacemaker, |
| | | moderate reduction of ejection fraction, ESRD |
| | | undergoing regularly scheduled dialysis, premature |
| | | infant PCA < 60 weeks, history (>3 months) of MI, |
| | | CVA, TIA, or CAD/stents. |
| ASA IV | A patient with severe | Examples include (but not limited to): recent (<3 |
| | systemic disease that is | months) MI, CVA, TIA, or CAD/stents, ongoing |
| | a constant threat to life | cardiac ischemia or severe valve dysfunction, severe |
| | | reduction of ejection fraction, sepsis, DIC, ARD or |
| | | ESRD not undergoing regularly scheduled dialysis |
| ASA V | A moribund patient who | Examples include (but not limited to): ruptured |
| | is not expected to | abdominal/thoracic aneurysm, massive trauma, |
| | survive without the | intracranial bleed with mass effect, ischemic bowel in |
| | operation | the face of significant cardiac pathology or multiple |
| | | organ/system dysfunction |
| ASA VI | A declared brain-dead | |
| | patient whose organs | |
| | are being removed for | |
| | donor purposes | |

If a patient was discharged, dropped out or died before all samples had been obtained, the patient wasn't excluded and the collected samples were analyzed unless the patient asked not to be included in the data analyses.

### Biomarker Measurements:

CT-pro ET-1 was measured using an immunofluorescence assay on BRAHMS Kryptor Compact plus (BRAHMS GmbH, Heringsdorf, Germany) with TRACE technology (Time Resolved Amplified Cryptate Emission). Two fluorescent tracers, a donor (Europium cryptate) and an acceptor (XL665), each bound to an antibody were used. When the antibodies bind to their antigen, CT-proET-1, the tracers are activated and energy is transferred from cryptate to XL665 and a signal at 665 nm is emitted. The fluorescence is proportional to the CT-proET1 concentration. The 99th percentile of CT-proET1 in a healthy population is 72.9 pmol/L, the lower analytical detection limit was 3 pmol/L with a reading zone up to 5000 pmol/L.

Cardiac troponin T (cnTNT) was analysed using a high sensitive electrochemiluminescence immunoassay on a Cobas e 602/cobas e 601/cobas e 411 (Roche Diagnostics Mannheim, Germany). Two antibodies specific for cTnT are used, one labeled with biotin, the other with a ruthenium complex that together forms a sandwich complex. Streptavidin is added to the sample before its moved to a measuring cell and unbound substances are eliminated by ProCell. An electric field is added causing electrochemical luminescence of 620 nm to be emitted by the ruthenium complex which is detected by a photomultiplier tube to determine the hsTnT concentration. The lower limit of detection for this assay is 5 ng/L, the 99th percentile of a healthy reference population is 14 ng/L.

PCT was measured using commercially available double sandwich immunoassay on a KRYPTOR platform (Thermo Fisher Scientific, Germany) with a measuring range of 0.02-5000 ng/ml, and a functional assay sensitivity and lower detection limit of at least 0.06 ng/ml and 0.02 ng/ml, respectively.

MR-proADM plasma concentrations were measured using Kryptor®, Thermo Fisher Scientific, Germany) with a limit of detection of 0.05 nmol/L.

### Statistical Analysis:

Data analyses were performed using Graphpad PRISM 8. Data distribution was reviewed using D'agostino & Pearson test. Comparisons between different groups were performed using fisher's exact test and Kruskal Wallis test. P-values<0.05 were considered significant.

### Example 1: Patient Characteristics

Patient characteristics upon study enrolment are summarized in Table 1.

A total of 387 patients that have undergone surgery were analysed. Enrolled patients had an average age of 70 years with a ratio of 44% woman and 56% men. Using American Society of Anesthesiologists Physical Status Score (ASAPS) 265 (68%) of the patients were classified as high-risk patients (ASA III-IV), whereas as 116 (30%) of the patients were classified as low-risk patients (ASA Of all patients 37 (9,6%) suffered postsurgically a major cardiovascular or cerebrovascular event (MACCE), 94 (24%) a myocardial injury after non-cardiac surgery (MINS), wherein 14 patients experience both. In regard to infection as a postsurgical adverse event 105 (27%) developed an infection of which 11 suffered of a blood infection. The overall mortality rate was 1.3% (5 deceased).

### Example 2: Correlation of a Postsurgical Occurrence of MACCE with the Biomarkers

Table 2 summarizes the patient characteristics in regard to the occurrence of MACCE. Biomarkers levels were determined for all patients preoperatively (preOP), on the post-anesthesia care unit (PACU) as well as one day (POD1), two days (POD2) and three days (POD3) after surgery.

FIGS. 1-3 summarize the results for MR-proADM, PCT and CT-proET-1. The levels of all biomarkers were elevated in the group of patients that suffered a MACCE after surgery. This trend is visible for all sample points preOP, PACU, as well as PODs 1-3.

For MR-proADM at all observed sample points a difference is visible, which becomes however more pronounced postsurgically, starting from PACU up to POD3 (FIG. 1). While the level of MR-proADM in MACCE patients is continuously rising from preOP over PACU up to POD1-3, the level of MR-proADM in NO MACCE patients is maintained constant or even decreases after POD1.

Similar results are seen for PCT as a marker for occurrence of MACCE (FIG. 2). Notably, starting at PACU a steady and steep 4-fold rise of the level of PCT up to POD3 is visible in patients that suffer of MACCE, while patients that did not experience MACCE show a mild increase at POD1, the level of which is maintained constant or decreasing for the subsequent days POD1-POD3.

For CT-proET-1 preoperatively and on the PACU the difference between MACCE and NO MACCE patients is particularly pronounced with p-values of 0.03 (preOP) and 0.0001 (PACU) (FIG. 3). However, also for later sample points a clear difference of the proET-1 level in regard to the incidence of MACCE can be established; the p-value for POD2 being 0,006 and for POD3 0.007. Notably, at sample points POD1-3 the level of CT-proET1 of NO MACCE patients declines, whereas in MACCE patients is maintained or even increases.

A single cut-off value for all sample points (preOP, PACU, POD1-3) of 84.7 pmol/L for CT-proET1 yielded a high statistical Fisher's exact test p=<0.0001 (see Table 3).

The results demonstrate that MR-proADM, CT-proET1 as well as PCT may serve for a reliable prediction or diagnosis for the incidence of MACCE in postsurgical patients. The prediction or diagnosis can be established with a single threshold value for all sample points or at individual sample points (e.g. preOP, PACU, POD1-3) by comparison sample point specific values.

In addition, the different temporal development of the level of the biomarkers in MACCE and NO MACCE patients allow for a further reliable prognosis or diagnosis, when taking samples at two time points or more and analyzing the ratio or absolute difference for the different sample points. While in MACCE patients the level of the biomarkers continuously rise, in NO MACCE patients after a mild increase postsurgically the level of the biomarkers remains steady or even decreases over the course of 72 hours postsurgically up to POD3.

### Example 3: Correlation of a Postsurgical Occurrence of MACCE with ECG Changes or Creatine Levels

In order to test the confidence of a prognosis or diagnosis for further markers or parameters at the time of analysis the ECGs of 146 patients were analysed. Table 4 presents the distribution of ECG-changes in patients with and without MACCE. The number of ECGs analysed may not be for a clear statistical statement, however the current distribution of ECG-changes does not seem to be a good predictor of MACCE as opposed to the biomarkers MR-proADM, PCT and/or CT-1.

Since renal function is known to affect CT-proET1 levels, creatinine levels were also analysed in relation to the incidence of MACCE. FIG. 4 shows that the Kruskal Wallis test revealed no significant difference in creatinine levels in patients with and without MACCE. Therefore, the finding that CT-proET1 levels or one of the other biomarkers predicts MACCE in the patients does not seem to be affected by the patient's renal function.

### Example 4: Correlation of a Postsurgical Occurrence of MINS with the Biomarkers

Table 5 summarizes the patient characteristics in regard to the occurrence of MINS. Biomarkers levels were determined for all patients preoperatively (preOP), on the post-anesthesia care unit (PACU) as well as one day (POD1), two days (POD2) and three days (POD3) after surgery.

FIG. 5-7 summarize the results for MR-proADM, PCT and CT-proET-1. The levels of all biomarkers were elevated in the group of patients that suffered a MINS after surgery. This trend is visible for all sample points preOP, PACU, as well as PODs 1-3.

For MR-proADM a difference of the level of MINS and NO MINS patients is visible for sample points early on at PACU and even preoperatively, the difference becomes more pronounced postsurgically from POD1 to POD3 (FIG. 5). While the level of MR-proADM in MACCE patients is continuously rising to POD1 and maintained at POD2 and POD3 at a high level of approximately 1.75 nmol/L, the level of MR-proADM in NO MACCE patients decreases steadily after POD1.

For PCT as a marker for occurrence of MINS similar results are established (FIG. 6). Notably, from PACU to POD1 the level rises 4-fold from approximately 0.25 μg/L to over 1 μg/L and continues to rise up to sample point POD3. On the contrary the level of PCT in NO MINS patients in postsurgical days 1 to three is maintained constant well below 1 μg/L, with a level of at POD3 being decrease in respect to POD1.

For CT-proET-1 the difference between MACCE and NO MACCE patients is particularly pronounced with p-values less than 0.001 for preOP and less than 0.0001 for PACU as well as POD1 through POD3. Notably, at sample points POD1-POD3 the level of CT-proET1 of NO MACCE patients declines, allowing for a further distinction from MACCE patients (FIG. 7).

The results demonstrate that MR-proADM, CT-proET1 as well as PCT may serve for a reliable prediction or diagnosis for the incidence of MINS in postsurgical patients. The prediction or diagnosis can be established with a single threshold value for all sample points or at individual sample points (e.g. preOP, PACU, POD1-3) by comparison to sample point specific values.

In addition, the different temporal development of the level of the biomarkers in MINS and NO MINS patients allow for a further reliable prognosis or diagnosis, when taking samples at two time points or more and analyzing the ratio or absolute difference for the different sample points.

### Example 5: Correlation of a Postsurgical Occurrence of any Infection with the Biomarkers

Table 6 summarizes the patient characteristics in regard to the occurrence of an infection. Biomarkers levels were determined for all patients preoperatively (preOP), on the post-anesthesia care unit (PACU) as well as one day (POD1), two days (POD2) and three days (POD3) after surgery.

FIG. 8-10 summarize the results for MR-proADM, PCT and CT-proET-1. The levels of all biomarkers, but in particular MR-proADM and PCT, were elevated in the group of patients that suffered an infection after surgery. This trend is visible for all sample points preOP, PACU, as well as PODs 1-3.

The level of MR-proADM for patients developing postsurgically an infection is elevated compared to the level in patients with no infection for sample points early on at PACU and even mildly preoperatively. The difference becomes more pronounced postsurgically from POD1 and in particular POD2 and POD3 (FIG. 8). While the level of MR-proADM in patients without any infection falls after POD1 from approximately 1.25 nmol/L to below 1.2 nmol/L at POD3. In patients having any infection the level is steadily maintained above 1.5 nmol/L for all PODs 1-3.

For PCT as a marker for occurrence of any infection a particular pronounced difference can be seen postsurgically from POD1 through POD3 (FIG. 9). While the level of PCT in postsurgical patients with no infection remains around or below 0.75 μg/L, the level of PCT in patients having any infection continuously rises from POD 1 (approximately 1.0 μg/L) to more than approximately 1.25 μg/L for PODs 2 and 3. Notably, also preoperatively and early on at PACU a mild difference is visible.

For CT-proET-1 the difference between patients with and without infections are less pronounced than for the markers PCT and MR-proADM. However, in particular at later sample points two or three days after surgery a visible difference allows for a reliable distinction (FIG. 10).

The results demonstrate that in particular MR-proADM and PCT, but possibly also CT-proET-1 may serve for a reliable prediction or diagnosis for the incidence of infection in postsurgical patients. The prediction or diagnosis can be established with a single threshold value for all sample points or at individual sample points (e.g. preOP, PACU, POD1-3) by comparison to sample point specific values.

In addition, the different temporal development of the level of the biomarkers in patients with and without any infection allow for a further reliable prognosis or diagnosis, when taking samples at at least two time points and analyzing the ratio or absolute difference for the different sample points.

### Example 6: Correlation of a Postsurgical Occurrence of a Blood Infection with the Biomarkers

Table 7 summarizes the patient characteristics in regard to the occurrence of a blood infection. Biomarkers levels were determined for all patients preoperatively (preOP), on the post-anesthesia care unit (PACU) as well as one day (POD1), two days (POD2) and three days (POD3) after surgery.

FIGS. 11-13 summarize the results for MR-proADM, PCT and CT-proET-1. The levels of all biomarkers, but in particular MR-proADM and PCT, were elevated in the group of patients that suffered a blood infection after surgery. This trend is visible for all sample points preOP, PACU, as well as PODs 1-3.

The level of MR-proADM for patients developing postsurgically a blood infection is elevated compared to the level in patients with no infection for sample points early on at PACU and notably even preoperatively. The difference becomes more pronounced postsurgically from POD1 to POD3 (FIG. 11). While the level of MR-proADM in patients without any infection falls after POD1 from approximately 1.3 nmol/L to below 1.2 nmol/L at POD3. For patients having a blood infection the level is steadily maintained above approximately 1.75 nmol/L for all PODs 1-3.

Similar to the incident of any infection for the occurrence of a blood infection, a particularly pronounced difference in the level of PCT can be seen postsurgically from POD1 through POD3 (FIG. 12). While the level of PCT in postsurgical patients with no blood infection remains around or below 0.75 μg/L, the level of PCT in patients having a blood infection continuously rises from POD 1 (approximately 2.0 μg/L) to more than approximately 2.5 μg/L for PODs 2 and 3. Notably, also preoperatively and early on at PACU a mild difference is visible.

For CT-proET-1 the difference between patients with and without a blood infection are less pronounced than for the markers PCT and MR-proADM. However, in particular at later sample points two or three days after surgery a visible difference allows for a distinction. While patients with a blood infection had at POD2 and 3 a level of CT-proET-1 of more than 100 pmol/L, the level of patients with no blood infection was at POD2 and POD3 around or below approximately 0.75 pmol/L (FIG. 13).

The results demonstrate that in particular MR-proADM and PCT, but possibly also CT-proET-1 may serve for a reliable prediction or diagnosis for the incidence of a blood infection in postsurgical patients. The prediction or diagnosis can be established with a single threshold value for all sample points or at individual sample points (e.g. preOP, PACU, POD1-3) by comparison to sample point specific values.

In addition, the different temporal development of the level of the biomarkers in patients with and without a blood infection allow for a further reliable prognosis or diagnosis, when taking samples at two time points or more and analyzing the ratio or absolute difference for the different sample points.

### Example 7: Correlation of a Postsurgical Occurrence of an Infection and MACCE with the Biomarkers

Table 8 summarizes the patient characteristics in regard to the postsurgical occurrence of an infection and MACCE. Biomarkers levels were determined for all patients preoperatively (preOP), on the post-anesthesia care unit (PACU) as well as one day (POD1), two days (POD2) and three days (POD3) after surgery.

FIGS. 14-19 summarize the results for MR-proADM, PCT and CT-proET-1.

Strikingly, the levels of all biomarkers were particularly elevated in the group of patients that suffered both an infection as well as a MACCE. This trend was clearly visible for all sample points preOP, PACU, as well as PODs 1-3.

For MR-proADM the level of patients that suffered of an infection and a MACCE exhibit clear differences to patients that did not suffer any one of these events or that suffered only one of the adverse events across all sample points (FIG. 14). The difference is particularly pronounced postsurgically. While patients that neither developed an infection nor experienced MACCE postsurgically showed at PACU or POD 1-3 a level of MR-proADM of 1 nmol/L or below, patients that suffered both adverse events showed a steady increase in the level of MR-proADM from approximately 1.5 nmol/L at PACU to approximately 2.3 nmol/L at POD3. Notably, the MR-proADM level of patients with a MACCE and any infection is also consistently higher than the MR-proADM level of patients with a MACCE, but no infection (FIG. 17). MR-proADM may thus serve as a reliable marker for the prognosis or diagnosis of either one as well as a combination of both postsurgical adverse events.

PCT appears to be a particularly valuable marker for the combined prognosis or diagnosis of infections or MACCE. This is particularly the case for sample points postsurgically from POD1 to POD3 (FIG. 15). The PCT levels of patients that developed an infection and suffered a MACCE events were at POD1 through POD3 consistently above 2 μg/L, averaging on POD2 and POD3 even at approximately 3 μg/L to 3.5 μg/L. On the contrary, for patients that postsurgically neither experienced a MACCE nor an infection, PCT levels of around or below 0.5 μg/L were determined. The more than 4-fold difference on POD1 and more than 6 to 7-fold difference on POD 2 or POD3 indicate a strong synergistic effect for the combined diagnosis or prediction of infection and MACCE using PCT as a marker. Notably, for postsurgical sample points the PCT level of patients with a MACCE and any infection is also found to be higher than the PCT level of patients with a MACCE, but no infection (FIG. 18).

Likewise, for CT-proET-1 across all sample points considerably higher levels were determined in patients suffering postsurgically both from an infection as well as a MACCE than in patients that experienced neither one of these adverse events (FIG. 16). While for the former group CT-proET-1 levels remained at or below approximately 80 pmol/L, in the latter group the level of CT-proET-1 was postsurgically from PACU to POD3 at or above approximately 125 pmol/L. Moreover, the CT-proET-1 level of patients with a MACCE and any infection is at any sample point presurgically or postsurgically higher than the CT-proET-1 level of patients with a MACCE, but no infection (FIG. 19). The data thus strongly support the prognostic and diagnostic ability of CT-proET-1 for either one as well as a combination of an infection or a MACCE as a postsurgical adverse event.

The data demonstrate that the prognosis and/or diagnosis of an infection and a MACCE using one of the described biomarkers can be achieved with a particularly high accuracy and reliability. From the predictive potential of the markers for one of the adverse events such a strong predictive power could not have been expected, pointing towards a functional synergy of these biomarkers in regard to the diagnosis or prediction of these distinct adverse events.

The prediction or diagnosis can be established with a single threshold value for all sample points or at individual sample points (e.g. preOP, PACU, POD1-3) by comparison to sample point specific values.

In addition, the different temporal development of the level of the biomarkers in patients with and without an infection and a MACCE allow for a further reliable prognosis or diagnosis, when taking samples at two time points or more and analyzing the ratio or absolute difference for the different sample points.

TABLE 1
Overall patient characteristics

| | | | No | | No | | No | Any | No Blood | Blood |
|---|---|---|---|---|---|---|---|---|---|---|
| | All | | MACCE | MACCE | MINS | MINS | Infection | Infection | Infection | Infection |
| No. of | 387 | | 350 | 37 | 293 | 94 | 282 | 105 | 376 | 11 |
| Patients | | | | | | | | | | |
| Avg Age | 70 | | 70 | 72 | | | 70 | 69 | 70 | 72 |
| Women | 44% | | 46% | 30% | | | 44% | 45% | 44% | 45% |
| Blood | 11 | (2.8%) | 6 | 5 | 10 | 1 | 0 | 11 | 0 | 11 |
| infection | | | | | | | | | | |
| Any | 105 | (27%) | 88 | 17 | 73 | 32 | 0 | 105 | 94 | 11 |
| infection | | | | | | | | | | |
| ASA I-II | 265 | (68%) | 243 | 21 | | | 199 | 66 | 260 | 5 |
| ASA III-IV | 116 | (30%) | 100 | 16 | | | 79 | 37 | 110 | 6 |
| MINS | 94 | (24%) | 80 | 14 | 0 | 94 | 62 | 32 | 93 | 1 |
| MACCE | 37 | (9.6) | 0 | 37 | 23 | 14 | 20 | 17 | 32 | 5 |
| Deceased | 5 | (1.3%) | 0 | 5 | | | 0 | 5 | 3 | 2 |

TABLE 2
Patient characteristics in regards to the incidence of MACCE

| | All | No MACCE | | MACCE | |
|---|---|---|---|---|---|
| No. of Patients | 387 | 350 | | 37 | (9.6%) |
| Avg Age | 70 | 70 | | 72 | |
| Women | 44% | 46% | | 30% | |
| Blood infection | 11 | 6 | | 5 | |
| Any infection | 105 | 88 | | 17 | |
| ASA I-II | 265 | 243 | | 21 | |
| ASA III-IV | 116 | 100 | (29%) | 16 | (43%) |
| MINS | 94 | 80 | (23%) | 14 | (38%) |

| | | | | |
|---|---|---|---|---|
| Deceased | 5 | 0 | 5 | (13.5%) |

TABLE 3
Fisher's exact test for an all sample
points threshold for CT-proET-1

| | no MACCE | MACCE |
|---|---|---|
| CT-proET1 ≥84.7 | 612 | 105 |
| CT-proET1 <84.7 | 955 | 61 |
| Total | 1,567 | 166 |

Fisher's exact test p =< 0,0001

TABLE 4
ECG-changes in patients with and without MACCE.

| | no MACCE | MACCE |
|---|---|---|
| ECG-changes | 24 | 1 |
| No ECG-changes | 108 | 13 |
| Total | 132 | 14 |

TABLE 5
Patient characteristics in regards to the incidence of MINS

| | All | No MINS | MINS | |
|---|---|---|---|---|
| No. of Patients | 387 | 293 | 94 | |
| Avg Age | 70 | 70 | 72 | |
| Women | 44% | 46% | 30% | |
| Blood infection | 11 | 10 | 1 | |
| Any infection | 105 | 73 | 32 | |
| MACCE | 37 | 23 (7.8%) | 14 | (15%) |
| Deceased | 5 | 0 | 5 | (5.3%) |

TABLE 6
Patient characteristics in regards to the incidence of any infection

| | All | | No Infection | | Any Infection | |
|---|---|---|---|---|---|---|
| No. of Patients | 387 | | 282 | | 105 | (27%) |
| Avg Age | 70 | | 70 | | 69 | |
| Women | 44% | | 44% | | 45% | |
| ASA I-II | 265 | | 199 | | 66 | |
| ASA III-IV | 116 | (30%) | 79 | (28%) | 37 | (35%) |
| MINS | 94 | (24%) | 62 | (22%) | 32 | (30.5%) |
| MACCE | 37 | (9.6%) | 20 | (7.1%) | 17 | (16.2%) |
| Deceased | 5 | (1.3%) | 0 | (0%) | 5 | (4.8%) |

TABLE 7
Patient characteristics in regards to
the incidence of a blood infection

| | All | | No Blood Inf | | Blood Inf | |
|---|---|---|---|---|---|---|
| No. of Patients | 387 | | 376 | | 11 | (2.8%) |
| Avg Age | 70 | | 70 | | 72 | |
| Women | 44% | | 44% | | 45% | |
| ASA I-II | 265 | | 260 | | 5 | |
| ASA III-IV | 116 | (30%) | 110 | (29%) | 6 | (55%) |
| MINS | 94 | (24%) | 93 | (25%) | 1 | (9.1%) |
| MACCE | 37 | (9.6%) | 32 | (8.5%) | 5 | (45.5%) |
| Deceased | 5 | (1.3%) | 3 | (0.8%) | 2 | (18.2%) |

TABLE 8
Patient characteristics in regards to
the incidence of an infection and MACCE

| No. of Patients | No infection | infection |
|---|---|---|
| No MACCE | 262 | 88 |
| MACCE | 20 | 17 |

TABLE 9
Definition of MACCE

| | |
|---|---|
| Non-fatal | An absence of cardiac rhythm or presence of chaotic rhythm requiring any |
| cardiac arrest | component of basic or advanced cardiac life support. |
| Acute | Increase and gradual decrease in troponin level or a faster increase and |
| myocardial | decrease if creatine kinase isoenzyme as marker of myocardial necrosis in |
| infarction | the company of at least one of the following: Ischemic symptoms, abnormal |
| | Q-waves on the ECG, ST-segment elevation or depression, coronary artery |
| | intervention (e.g. coronary angioplasty) or a atypical decrease in an |
| | elevated troponin level detected at its peak after surgery in a patient without |
| | documented alternative explanation for the troponin elevation. |
| Congestive | New in-hospital signs or symptoms of dyspnoea or fatigue, orthopnoea, |
| heart failure | paroxysmal nocturnal dyspnoea, increased jugular venous pressure, |
| | pulmonary rales on physical examination, cardiomegaly or pulmonary |
| | vascular engorgement. |
| New cardiac | ECG evidence of atrial flutter, atrial fibrillation, or second- or third- degree |
| arrhythmia | atrioventricular conduction block. |
| Angina | Dull diffuse substernal chest discomfort precipitated by exertion or emotion |
| | and relieved by rest or glyceryl trinitrate. |
| Stroke | Embolic, thrombotic, or hemorrhagic event lasting at least 30 in with or |
| | without persistent residual motor, sensory, or cognitive dysfunction; if the |
| | neurological symptoms continue for >24 h, a person is diagnosed with |
| | stroke, and if lasting <24 h the event is defined as a transient ischemic |
| | attack. |

TABLE
10 Ischemic ECG manifestations

| | |
|---|---|
| Pathological | Development of pathologic Q waves ≥30 ms |
| Q-waves | in any two contiguous leads |
| LBBB | Development of left bundle branch block |
| ST-elevation | Development of ST-segment elevation ≥2 mm |
| | in V₁, V₂, V₃ or ≥1 mm in other leads |
| ST-depression | Development of ST-segment depression ≥1 mm |
| T-wave | Symmetric inversion of T-waves ≥1 mm in at |
| inversion | least two contiguous leads |

### REFERENCES

Albrich W C, Dusemund F, Ruegger K, et al. (2011a) Enhancement of CURB65 score with proadrenomedullin (CURB65-A) for outcome prediction in lower respiratory tract infections: derivation of a clinical algorithm. BMC Infect Dis; 11:112.
Albrich W C, Ruegger K, Dusemund F, et al. (2011 b) Optimised patient transfer using an innovative multidisciplinary assessment in Kanton Aargau (OPTIMA I): an observational survey in lower respiratory tract infections. Swiss Med Wkly.; 141:w13237.
Albrich W C, Ruegger K, Dusemund F, et al. (2013) Biomarker-enhanced triage in respiratory infections: a proof-of-concept feasibility trial. Eur Respir J.; 42(4):1064-1075.
Andaluz-Ojeda D, Cicuendez R, Calvo D, et al. (2015) Sustained value of proadrenomedullin as mortality predictor in severe sepsis. J Infect. 71(1):136-139.
Benemei et al. CGRP receptors in the control of pain and inflammation, Current Opinion in Pharmacology Volume 9, Issue 1, February 2009, Pages 9-14)
Bhandari, S. S. et al. (2014) ‘Plasma C-terminal proEndothelin-1 (CTproET-1) is affected by age, renal function, left atrial size and diastolic blood pressure in healthy subjects’, Peptides, 52, pp. 53-57. doi: 10.1016/j.peptides.2013.12.001.
Bone et al. (1992), Definitions for sepsis and organ failure and guidelines for the use of innovative therapies in sepsis. The ACCP/SCCM Consensus Conference Committee. American College of Chest Physicians/Society of Critical Care Medicine. CHEST 101(6): 1644-55.
Botto, F. et al. (2014) ‘Myocardial Injury after Noncardiac Surgery: A Large, International, Prospective Cohort Study Establishing Diagnostic Criteria, Characteristics, Predictors, and 30-day Outcomes’, Anesthesiology, 120(3), pp. 564-578. doi: 10.1097/ALN.0000000000000113.
Devereaux, P. J. and Sessler, D. I. (2015) ‘Cardiac Complications in Patients Undergoing Major Noncardiac Surgery’, New England journal of medicine, p. 2258.
Devereaux, P. J. et al. (2005) ‘Surveillance and prevention of major perioperative ischemic cardiac events in patients undergoing noncardiac surgery: a review’, CMAJ: Canadian Medical Association Journal, 173(7), pp. 779-788. doi: 10.1503/cmaj.050316.
Devereaux, P. J. et al. (2008) ‘Effects of extended-release metoprolol succinate in patients undergoing non-cardiac surgery (POISE trial): a randomised controlled trial’, LANCET, 371(9627), pp. 1839-1847. doi: 10.1016/S0140-6736(08)60601-7.
Devereaux, P. J. et al. (2017) ‘Association of Postoperative High-Sensitivity Troponin Levels With Myocardial Injury and 30-Day Mortality Among Patients Undergoing Noncardiac Surgery’, JAMA, 317(16), pp. 1642-1651. doi: 10.1001/jama.2017.4360.
Doyle D J, Garmon E H. (2018) American Society of Anesthesiologists Classification (ASA Class) [Updated 2018 Oct. 27]. In: StatPearls [Internet]. Treasure Island (FL): StatPearls Publishing; 2018 January. Available from: https://www.ncbi.nlm.nih.gov/books/NBK441940/.
Ekeloef, S. et al. (2016) ‘Troponin elevations after non-cardiac, non-vascular surgery are predictive of major adverse cardiac events and mortality: a systematic review and meta-analysis’, BRITISH JOURNAL OF ANAESTHESIA, 117(5), pp. 559-568. doi: 10.1093/bja/aew321.
Haaf, P. et al. (2014) ‘Clinical Research: Utility of C-terminal Proendothelin in the Early Diagnosis and Risk Stratification of Patients With Suspected Acute Myocardial Infarction’, Canadian Journal of Cardiology, 30, pp. 195-203. doi: 10.1016/j.cjca.2013.11.020.
Hanson, I. et al. (2013) ‘Angiographic and Clinical Characteristics of Type 1 Versus Type 2 Perioperative Myocardial Infarction’, CATHETERIZATION AND CARDIOVASCULAR INTERVENTIONS, 82(4), pp. 622-628. doi: 10.1002/ccd.24626.
Hartmann O, Schuetz P, Albrich W C, Anker S D, Mueller B, Schmidt T (2012. Time-dependent Cox regression: serial measurement of the cardiovascular biomarker proadrenomedullin improves survival prediction in patients with lower respiratory tract infection. Int J Cardiol.; 161(3):166-173.
Horan T C, Andrus M, Dudeck M A (2008). CDC/NHSN surveillance definition of health care-associated infection and criteria for specific types of infections in the acute care setting. Am J Infect Control.; 36(5):309-332.)
Joyce et al. (1990) Calcitonin gene-related peptide levels are elevated in patients with sepsis. Surgery. 108(6):1097-101)
Kaukonen K M, Bailey M, Suzuki S, Pilcher D, Bellomo R. (2014) Mortality related to severe sepsis and septic shock among critically ill patients in Australia and New Zealand, 2000-2012. JAMA.; 311(13):1308-1316.
Kitamura K et al. (1993) Cloning and characterization of cDNA encoding a precursor for human adrenomedullin. Biochem Biophys Res Commun.
Landesberg, G. et al. (2009) ‘Perioperative Myocardial Infarction’, CIRCULATION, 119(22), pp. 2936-2944. doi: 10.1161/CIRCULATIONAHA.108.828228.
Levy M M et al. 2001 SCCM/ESICM/ACCP/ATS/SIS International Sepsis Definitions Conference. Crit Care Med. 2003 April; 31(4):1250-6).
Lundberg, O. H. M. et al. (2016) ‘Adrenomedullin and endothelin-1 are associated with myocardial injury and death in septic shock patients’, CRITICAL CARE, 20. doi: 10.1186/s13054-016-1361-y.
Martin G S, Mannino D M, Eaton S, Moss M. (2003) The epidemiology of sepsis in the United States from 1979 through 2000. N Engl J Med.; 348(16):1546-1554.
Mauermann, E., Buse, G. L. and Puelacher, C. (2016) ‘Myocardial injury after noncardiac surgery: An underappreciated problem and current challenges’, Current Opinion in Anaesthesiology, 29(3), pp. 403-412. doi: 10.1097/ACO.0000000000000336.
Miskovic at al. (2017) Postoperative pulmonary complications, British Journal of Anaesthesia, 118 (3): 317-34.
Nikitenko et al. (2006) Adrenomedullin and CGRP interact with endogenous calcitonin-receptor-like receptor in endothelial cells and induce its desensitisation by different mechanisms Journal of Cell Science 119, 910-922.
Noordzij, P. G. et al. (2015) ‘High-sensitive cardiac troponin T measurements in prediction of non-cardiac complications after major abdominal surgery’, BJA: The British Journal of Anaesthesia, 114(6), pp. 909-918. doi: 10.1093/bja/aev027.
Papassotiriou, J. et al. (2006) ‘Immunoluminometric assay for measurement of the C-terminal endothelin-1 precursor fragment in human plasma’, CLINICAL CHEMISTRY, 52(6), pp. 1144-1151. doi: 10.1373/clinchem.2005.065581.
Parlapiano et al. (1999) CGRP and ET-1 plasma levels in normal subjects, Eur Rev Med Pharmacol Sci.; 3(3):139-41
Protopapa, K. L. et al. (2014) ‘Development and validation of the Surgical Outcome Risk Tool (SORT)’, British Journal of Surgery, 101(13), pp. 1774-1783. doi: 10.1002/bjs.9638.
Puelacher et al (2015) Perioperative Myocardial Injury After Noncardiac Surgery. Circulation. 2017; 137:1221-1232
Riera J, Senna A, Cubero M, Roman A, Rello J, (2015) Study Group Investigators TV. Primary Graft Dysfunction and Mortality Following Lung Transplantation: A Role for Proadrenomedullin Plasma Levels. Am J Transplant.
Sabaté, S. et al. (2011) ‘Incidence and predictors of major perioperative adverse cardiac and cerebrovascular events in non-cardiac surgery’, BRITISH JOURNAL OF ANAESTHESIA, p. 879.
Schoe A, Schippers E F, Struck J, et al. (2015 Postoperative pro-adrenomedullin levels predict mortality in thoracic surgery patients: comparison with Acute Physiology and Chronic Health Evaluation IV Score*. Crit Care Med.; 43(2):373-381.
Sessler, D. I. and Khanna, A. K. (2018) ‘Perioperative myocardial injury and the contribution of hypotension’, INTENSIVE CARE MEDICINE, 44(6), pp. 811-822. doi: 10.1007/s00134-018-5224-7.
Setianto, B. Y. et al. (2016) ‘On-admission high endothelin-1 level independently predicts in-hospital adverse cardiac events following ST-elevation acute myocardial infarction’, International Journal of Cardiology, 220, pp. 72-76. doi: 10.1016/j.ijcard.2016.06.071.
Sexton et al. (2008) Procalcitonin has bioactivity at calcitonin receptor family complexes: Potential mediator implications in sepsis, Crit. Care Med Vol. 36, No. 5.
Singer M, Deutschman C S, Seymour C W, et al. (2016) The Third International Consensus Definitions for Sepsis and Septic Shock (Sepsis-3). JAMA. Feb. 23 2016; 315(8):801-810.
Smith D. M. et al. (2002) Adrenomedullin: receptor and signal transduction, Biochemical Society Transactions Aug. 1, 2002, 30 (4) 432-437
Thygesen, K. et al. (2012) ‘Third Universal Definition of Myocardial Infarction’, CIRCULATION, 126(16), p. 2020-+. doi: 10.1161/CIR.0b013e31826e1058.
Vincent J L, Sakr Y, Sprung C L, et al. (2006) Sepsis in European intensive care units: results of the SOAP study. Crit Care Med. 34(2):344-353.
Wang et al. (2004) Prevention of endothelin-1-induced increases in blood pressure: role of endogenous CGRP, Am J Physiol Heart Circ Physiol 287: H1868-H1874.
Weiser T G et al. (2015) Estimate of the global volume of surgery in 2012: an assessment supporting improved health outcomes. Lancet 2015; 385(suppl2):S11.
Whicher, J., Bienvenu, J., & Monneret, G. (2001). Procalcitonin as an Acute Phase Marker. Annals of Clinical Biochemistry, 38(5), 483-493.
Zhang, C.-L. et al. (2017) ‘Plasma endothelin-1-related peptides as the prognostic biomarkers for heart failure A PRISMA-compliant meta-analysis’, MEDICINE, 96(50). doi: 10.1097/MD.0000000000009342.

1. Method for the diagnosis, prognosis, risk assessment and/or risk stratification of an adverse event in the health of a postsurgical patient, comprising
providing at least one sample of a patient, who has undergone, is undergoing or will undergo surgery, wherein the at least one sample has been isolated from the patient presurgically, perioperative or postsurgically,
determining a level of at least one biomarker in said at least one sample,
wherein said level of the at least one biomarker or fragment(s) thereof correlates with the likelihood of said adverse event, wherein the at least one biomarker is proADM or fragment(s) thereof and the adverse event is an infection.

2. Method according to claim 1, wherein the adverse event is a fungal, a bacterial or a viral infection.

3. Method according to claim 1 or 2, wherein the infection is a blood infection, a respiratory tract infection, a urinary tract infection, a skin infection or an abdominal cavity infection.

4. Method according to any one of the preceding claims, wherein the adverse event is a blood stream infection, sepsis, severe sepsis and/or septic shock.

5. Method according to claim 1, wherein the adverse event is a cardiovascular or cerebrovascular event and an infection.

6. Method according to claim 1, wherein the adverse event is a myocardial injury after non-cardiac surgery (MINS) and an infection.

7. Method according to claim 1, wherein the adverse event is a major cardiovascular or cerebrovascular event (MACCE) and an infection.

8. Method according to claim 1, wherein the adverse event is a cardiovascular or cerebrovascular event and a blood infection.

9. Method according to claim 1, wherein the adverse event is a myocardial injury after non-cardiac surgery (MINS) and a blood infection.

10. Method according to claim 1, wherein the adverse event is a major cardiovascular or cerebrovascular event (MACCE) and a blood infection.

11. Method according to any one of the preceding claims, wherein a first sample is isolated from the patient at a first time point and a second sample is isolated from the patient at a second time point, wherein preferably between the first and the second time point at least 6 hours, 12 hours, 24 hours, 36 hours, 48 hours or more have passed.

12. Method according to any one of the preceding claims, a first sample is isolated from the patient at a first time point and a second sample is isolated from the patient at a second time point, wherein the likelihood of an adverse event correlates with the absolute difference, the ratio and/or the rate of change of the level of said biomarker in regards to the first and second time point.

13. Method according to any one of the preceding claims, wherein a first sample is isolated from the patient postsurgically at a first time point and a second sample is isolated from the patient postsurgically at a second time point, wherein preferably between the first and second time point at least 12 hours, 24 hours, 36 hours, 48 hours or more have passed and an increase or a leveling in the level of proADM or fragment(s) thereof indicates an elevated likelihood of the adverse event.

14. Method according to any one of the preceding claims, wherein determining a level of proADM or fragment(s) thereof comprises determining a level of MR-proADM or mature ADM in the sample.

15. Method according to any one of the preceding claims, additionally comprising a therapy guidance, stratification and/or control.

16. Method according to any one of the preceding claims, wherein the level of the at least one biomarker correlates with the likelihood of an infection that is indicative of initiation of a treatment with an anti-infective agent, preferably an antibiotic agent.

17. Method according to any one of the preceding claims, wherein the level of the at least one biomarker correlates with the likelihood of a postsurgical adverse event that is indicative of the patient requiring frequent or increased level of monitoring and/or critical care.

18. Method according to any one of the preceding claims, wherein determining a level of at least one biomarker comprises determining the level of proADM or fragment(s) thereof and a level of proADM or fragment(s) thereof above a threshold value indicates an elevated likelihood of an infection.

19. Method according to any one of the preceding claims, wherein determining a level of at least one biomarker comprises determining the level of proADM or fragment(s) thereof and a level of proADM or fragment(s) thereof above a threshold value indicates an elevated likelihood of a blood infection.

20. Method according to any one of the preceding claims, wherein determining a level of at least one biomarker comprises determining the level of proADM or fragment(s) thereof and a level of proADM or fragment(s) thereof above a threshold value indicates an elevated likelihood of an infection, preferably a blood infection, and an adverse cardiovascular or cerebrovascular event, preferably a MACCE, PMI and/or a MINS.

21. Kit for carrying out the method according to any one of the preceding claims, wherein the kit comprises
detection reagents for determining the level of at least one biomarker, wherein the at least one biomarker is proADM or fragment(s) thereof and
reference data for the likelihood of a postsurgical adverse event, in particular reference data for threshold or cut-off value(s), wherein said reference data is preferably stored on a computer readable medium and/or employed in the form of computer executable code configured for comparing the determined levels of proADM or fragment(s) thereof with the threshold or cut-off value(s), wherein the adverse event is an infection.

22. Method for the diagnosis, prognosis, risk assessment and/or risk stratification of an adverse event in the health of a postsurgical patient, comprising
providing at least one sample of a patient, who has undergone, is undergoing or will undergo surgery, wherein the at least one sample has been isolated from the patient presurgically, perioperative or postsurgically,
determining a level of at least one biomarker in said at least one sample,
wherein said level of the at least one biomarker or fragment(s) thereof correlates with the likelihood of said adverse event, wherein the at least one biomarker is PCT or fragment(s) thereof and the adverse event is a cardiovascular or cerebrovascular event.

23. Method according to claim 22, wherein the cardiovascular event is a myocardial infarction (MI).

24. Method according to claim 22, wherein the cardiovascular event is a myocardial injury after non-cardiac surgery (MINS).

25. Method according to claim 22, wherein the cardiovascular event is a major adverse cardiovascular or cerebrovascular event (MACCE).

26. Method according to claim 22, wherein the adverse event is a perioperative myocardial injury (PMI)

27. Method according to claim 22, wherein the cerebrovascular event is a stroke and/or a transient ischemic attack.

28. Method according to claim 22, wherein the adverse event is a cardiovascular or cerebrovascular event and an infection.

29. Method according to claim 22, wherein the adverse event is a myocardial injury after non-cardiac surgery (MINS) and an infection.

30. Method according to claim 22, wherein the adverse event is a major cardiovascular or cerebrovascular event (MACCE) and an infection.

31. Method according to claim 22, wherein the adverse event is a cardiovascular or cerebrovascular event and a blood infection.

32. Method according to claim 22, wherein the adverse event is a myocardial injury after non-cardiac surgery (MINS) and a blood infection.

33. Method according to claim 22, wherein the adverse event is a major cardiovascular or cerebrovascular event (MACCE) and a blood infection.

34. Method according to any one of the preceding claims 22-33, wherein a first sample is isolated from the patient at a first time point and a second sample is isolated from the patient at a second time point, wherein preferably between the first and the second time point at least 6 hours, 12 hours, 24 hours, 36 hours, 48 hours or more have passed.

35. Method according to any one of the preceding claims 22-34, a first sample is isolated from the patient at a first time point and a second sample is isolated from the patient at a second time point, wherein the likelihood of an adverse event correlates with the absolute difference, the ratio and/or the rate of change of the level of said biomarker in regards to the first and second time point.

36. Method according to any one of the preceding claims 22-35, wherein a first sample is isolated from the patient postsurgically at a first time point and a second sample is isolated from the patient postsurgically at a second time point, wherein preferably between the first and second time point at least 12 hours, 24 hours, 36 hours, 48 hours or more have passed and an increase or a leveling in the level of PCT or fragment(s) thereof indicates an elevated likelihood of the adverse event.

37. Method according to any one of the preceding claims 22-36, wherein determining a level of PCT or fragment(s) thereof comprises determining a level of PCT 1-116, PCT 2-116 or PCT 3-116 in the sample.

38. Method according to any one of the preceding claims 22-37, additionally comprising a therapy guidance, stratification and/or control.

39. Method according to any one of the preceding claims 22-38, wherein the level of the at least one biomarker correlates with the likelihood of an adverse cardiovascular or cerebrovascular event that is indicative of further diagnostic assessment selected from the group consisting of a CT scan, an MRI scan, an angiogram, an arteriography, an X-Ray, an Echocardiogram, an ECG and an EEG.

40. Method according to any one of the preceding claims 22-39, wherein the level of the at least one biomarker correlates with the likelihood of an adverse cardiovascular or cerebrovascular event that is indicative of an initiation or preparation of an anticoagulation therapy, an oxygen therapy, a lysis therapy, a percutaneous coronary intervention, a percutaneous transluminal angioplasty, a coronary artery bypass graft and/or a stent implantation

41. Method according to any one of the preceding claims 22-40, wherein the level of the at least one biomarker correlates with the likelihood of a postsurgical adverse event that is indicative of the patient requiring frequent or increased level of monitoring and/or critical care.

42. Method according to any one of the preceding claims 22-41, wherein determining a level of at least one biomarker comprises determining the level of PCT or fragment(s) thereof and a level of PCT or fragment(s) thereof above a threshold value indicates an elevated likelihood of an adverse cardiovascular or cerebrovascular event, preferably a MACCE.

43. Method according to any one of the preceding claims 22-42, wherein determining a level of at least one biomarker comprises determining the level of PCT or fragment(s) thereof and a level of PCT or fragment(s) thereof above a threshold value indicates an elevated likelihood of an adverse cardiovascular or cerebrovascular event, preferably a MINS.

44. Method according to any one of the preceding claims 22-43, wherein determining a level of at least one biomarker comprises determining the level of PCT or fragment(s) thereof and a level of PCT or fragment(s) thereof above a threshold value indicates an elevated likelihood of an adverse cardiovascular or cerebrovascular event, preferably a PMI.

45. Method according to any one of the preceding claims 22-44, wherein determining a level of at least one biomarker comprises determining the level of PCT or fragment(s) thereof and a level of PCT or fragment(s) thereof above a threshold value indicates an elevated likelihood of an infection, preferably a blood infection, and an adverse cardiovascular or cerebrovascular event, preferably a MACCE, PMI and/or a MINS.

46. Kit for carrying out the method according to any one of the preceding claims 22-45, wherein the kit comprises
detection reagents for determining the level of at least one biomarker, wherein the at least one biomarker is PCT or fragment(s) thereof and
reference data for the likelihood of a postsurgical adverse event, in particular reference data for threshold or cut-off value(s), wherein said reference data is preferably stored on a computer readable medium and/or employed in the form of computer executable code configured for comparing the determined levels of the least one biomarker selected from the group consisting of PCT or fragment(s) thereof with the threshold or cut-off value(s), wherein the adverse event is a cardiovascular or cerebrovascular event.

47. Method for the diagnosis, prognosis, risk assessment and/or risk stratification of an adverse event in the health of a postsurgical patient, comprising
providing at least one sample of a patient, who has undergone, is undergoing or will undergo surgery, wherein the at least one sample has been isolated from the patient presurgically, perioperative or postsurgically,
determining a level of at least one biomarker in said at least one sample,
wherein said level of the at least one biomarker or fragment(s) thereof correlates with the likelihood of said adverse event, wherein the at least one biomarker is proET-1 or fragment(s).

48. Method according to claim 47, wherein the adverse event is a cardiovascular or cerebrovascular event.

49. Method according to claim 47, wherein the cardiovascular event is a myocardial infarction (MI).

50. Method according to claim 47, wherein the cardiovascular event is a myocardial injury after non-cardiac surgery (MINS).

51. Method according to claim 47, wherein the cardiovascular event is a major adverse cardiovascular or cerebrovascular event (MACCE).

52. Method according to claim 47, wherein the adverse event is a perioperative myocardial injury (PMI)

53. Method according to claim 47, wherein the cerebrovascular event is a stroke and/or a transient ischemic attack.

54. Method according to claim 47, wherein the adverse event is an infection.

55. Method according to claim 47, wherein the adverse event is a fungal, a bacterial or a viral infection.

56. Method according to claim 54 or 55, wherein the infection is a blood infection, a respiratory tract infection, a urinary tract infection, a skin infection or an abdominal cavity infection.

57. Method according to any one of claims 54-56, wherein the adverse event is a blood stream infection, sepsis, severe sepsis and/or septic shock.

58. Method according to claim 54-57, wherein the adverse event is a cardiovascular or cerebrovascular event and an infection.

59. Method according to claim 54-58, wherein the adverse event is a myocardial injury after non-cardiac surgery (MINS) and an infection.

60. Method according to claim 54-59, wherein the adverse event is a major cardiovascular or cerebrovascular event (MACCE) and an infection.

61. Method according to claim 48-53, wherein the adverse event is a cardiovascular or cerebrovascular event and a blood infection.

62. Method according to claim 47, wherein the adverse event is a myocardial injury after non-cardiac surgery (MINS) and a blood infection.

63. Method according to claim 47, wherein the adverse event is a major cardiovascular or cerebrovascular event (MACCE) and a blood infection.

64. Method according to any one of the preceding claims 47-63, wherein a first sample is isolated from the patient at a first time point and a second sample is isolated from the patient at a second time point, wherein preferably between the first and the second time point at least 6 hours, 12 hours, 24 hours, 36 hours, 48 hours or more have passed.

65. Method according to any one of the preceding claims 47-64, a first sample is isolated from the patient at a first time point and a second sample is isolated from the patient at a second time point, wherein the likelihood of an adverse event correlates with the absolute difference, the ratio and/or the rate of change of the level of said biomarker in regards to the first and second time point.

66. Method according to any one of the preceding claims 47-65, wherein a first sample is isolated from the patient postsurgically at a first time point and a second sample is isolated from the patient postsurgically at a second time point, wherein preferably between the first and second time point at least 12 hours, 24 hours, 36 hours, 48 hours or more have passed and an increase or a leveling in the level of proET-1 or fragment(s) thereof indicates an elevated likelihood of the adverse event.

67. Method according to any one of the preceding claims 47-66, wherein determining a level of proET-1 or fragment(s) thereof comprises determining a level of CT-proET-1 in the sample.

68. Method according to any one of the preceding claims 47-67, additionally comprising a therapy guidance, stratification and/or control.

69. Method according to any one of the preceding claims 47-68, wherein the level of the at least one biomarker correlates with the likelihood of an infection that is indicative of initiation of a treatment with an anti-infective agent, preferably an antibiotic agent.

70. Method according to any one of the preceding claims 47-69, wherein the level of the at least one biomarker correlates with the likelihood of an adverse cardiovascular or cerebrovascular event that is indicative of further diagnostic assessment selected from the group consisting of a CT scan, an MRI scan, an angiogram, an arteriography, an X-Ray, an Echocardiogram, an ECG and an EEG.

71. Method according to any one of the preceding claims 47-70, wherein the level of the at least one biomarker correlates with the likelihood of an adverse cardiovascular or cerebrovascular event that is indicative of an initiation or preparation of an anticoagulation therapy, an oxygen therapy, a lysis therapy, a percutaneous coronary intervention, a percutaneous transluminal angioplasty, a coronary artery bypass graft and/or a stent implantation

72. Method according to any one of the preceding claims 47-71, wherein the level of the at least one biomarker correlates with the likelihood of a postsurgical adverse event that is indicative of the patient requiring frequent or increased level of monitoring and/or critical care.

73. Method according to any one of the preceding claims 47-72, wherein determining a level of at least one biomarker comprises determining the level of proET-1 or fragment(s) thereof and a level of proET-1 or fragment(s) thereof above a threshold value indicates an elevated likelihood of an adverse cardiovascular or cerebrovascular event, preferably a MACCE.

74. Method according to any one of the preceding claims 47-73, wherein determining a level of at least one biomarker comprises determining the level of proET-1 or fragment(s) thereof and a level of proET-1 or fragment(s) thereof above a threshold value indicates an elevated likelihood of an adverse cardiovascular or cerebrovascular event, preferably a MINS.

75. Method according to any one of the preceding claims 47-74, wherein determining a level of at least one biomarker comprises determining the level of proET-1 or fragment(s) thereof and a level of proET-1 or fragment(s) thereof above a threshold value indicates an elevated likelihood of an adverse cardiovascular or cerebrovascular event, preferably a PMI.

76. Method according to any one of the preceding claims 47-75, wherein determining a level of at least one biomarker comprises determining the level of proET-1 or fragment(s) thereof and a level of proET-1 or fragment(s) thereof above a threshold value indicates an elevated likelihood of an infection.

77. Method according to any one of the preceding claims 47-76, wherein determining a level of at least one biomarker comprises determining the level of proET-1 or fragment(s) thereof and a level of proET-1 or fragment(s) thereof above a threshold value indicates an elevated likelihood of a blood infection.

78. Method according to any one of the preceding claims 47-77, wherein determining a level of at least one biomarker comprises determining the level of proET-1 or fragment(s) thereof and a level of proET-1 or fragment(s) thereof above a threshold value indicates an elevated likelihood of an infection, preferably a blood infection, and an adverse cardiovascular or cerebrovascular event, preferably a MACCE, PMI and/or a MINS.

79. Kit for carrying out the method according to any one of the preceding claims 47-78, wherein the kit comprises
detection reagents for determining the level of at least one biomarker, wherein the at least one biomarker is proET-1 or fragment(s) thereof, and
reference data for the likelihood of a postsurgical adverse event, in particular reference data for threshold or cut-off value(s), wherein said reference data is preferably stored on a computer readable medium and/or employed in the form of computer executable code configured for comparing the determined levels of the least one biomarker with the threshold or cut-off value(s).

80. Method according to any one of the preceding claims, wherein the patient has undergone, is undergoing or will undergo a non-cardiac surgery.

81. Method according to any one of the preceding claims, wherein the patient has undergone, is undergoing or will undergo an abdominal surgery.

82. Method according to any one of the preceding claims, wherein the patient is 50 years or older.

83. Method according to any one of the preceding claims, wherein the sample is isolated presurgically within 7 days, preferably within 3 days, one day, 12 hours or less before the surgery.

84. Method according to any one of the preceding claims, wherein the sample is isolated from the patients postsurgically at least 12 hours, 24 hours, 48 hours, 72 hours or more after surgery, and/or within 72 hours, 48 hours, 24 hours, 12 hours or less after the surgery.

85. Method according to any one of the preceding claims, wherein the sample is isolated postsurgically within 12 hours, preferably within 6 hours after surgery.

86. Method according to any one of the preceding claims, wherein the sample is isolated one day after surgery.

87. Method according to any one of the preceding claims, wherein the sample is isolated two days after surgery.

88. Method according to any one of the preceding claims, wherein the sample is isolated three days after surgery.

89. Method according to any one of the preceding claims, wherein a first sample is isolated presurgically and/or perioperatively and a second sample is isolated postsurgically.

90. Method according to any one of the preceding claims, wherein a first sample is isolated presurgically and a second sample is isolated perioperatively and/or postsurgically.

91. Method according to any one of the preceding claims, wherein the sample is selected from the group consisting of a blood sample, such as a whole blood sample, a serum sample or a plasma sample, a saliva sample and/or a urine sample.
